(19) United States
(12) Patent Application Publication
(10) Pub. No.: US 2009/0202565 A1
(43) Pub. Date: Aug. 13, 2009

(54) TORC POLYNUCLEOTIDES AND POLYPEPTIDES AND METHOD OF USE

(76) Inventors: Mark Aron Labow, Lexington, MA (US); Mark Bittinger, West Roxbury, MA (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/577,961

(22) PCT Filed: Oct. 24, 2005
(86) PCT No.: PCT/US05/38207

§ 371 Date: Mar. 19, 2009

Related U.S. Application Data
(60) Provisional application No. 60/621,716, filed on Oct. 25, 2004.

(51) Int. Cl. A61K 39/395; C12Q 1/68; C12Q 1/48; C12Q 1/02; G01N 33/566; A61K 38/02; A61K 31/7105; A61K 31/7088

(52) U.S. Cl. 424/172.1; 435/6; 435/15; 435/29; 435/375; 436/501; 514/2

(57) ABSTRACT

The present invention relates to a broad range of methods that utilize a transducer of regulated CREB (TORC)-related polynucleotide, polypeptide, or TORC-specific antibody. In addition the invention relates to TORC-related polynucleotide, polypeptide, or TORC-specific antibody compositions, including variants of TORC wild-type sequences. Exemplary methods include a method of stimulating a TORC related process in a cell as well as a method of inhibiting a TORC-related process in a cell, and a method of inhibiting TORC-related processes in a cell. The invention additionally discloses therapeutic methods of substantially inhibiting the development of, treating, or ameliorating a disease or pathological condition in a subject related to an abnormal level of a TORC-activated process in a cell that includes administering one or more therapeutically effective doses to the subject of either a substance that modulates accumulation of a TORC polypeptide in a subcellular region of the cell, or of a substance that inhibits expression of a TORC polypeptide in the cell. In an additional aspect a method of identifying an agent that modulates the activity of a TORC-related process in a cell is disclosed. In still a further aspect the invention relates to a method of detecting the presence or quantifying the amount of a TORC polypeptide in a sample. In a further aspect, a method is disclosed of determining whether the amount of a TORC polypeptide in a sample differs from the amount of the TORC polypeptide in a reference. An additional aspect relates to a method of contributing to the diagnosis or prognosis of, or to developing a therapeutic strategy for, a disease or pathology in a first subject, wherein the subcellular localization of a TORC polypeptide in the pathology is known to differ from the subcellular localization of the TORC polypeptide in a nonpathological state.

### FIELD OF THE INVENTION

The present invention relates generally to certain polynucleotides and polypeptides, cells harboring them, and methods of using them. In particular, TORC polynucleotides and TORC polypeptides, variants thereof, TORC-specific antibodies, and TORC-containing cells are disclosed. Pharmaceutical research methods, assay methods, diagnostic methods and therapeutic methods using TORC compositions are also disclosed.

### BACKGROUND OF THE INVENTION

The cyclic-AMP response element (CRE) binding protein (CREB) family of transcription factors represent a small group of proteins including CREB1 and the closely related CREM and ATF-1 proteins. The CREB1 protein controls gene expression by binding to the cAMP response element (CRE) present in the promoters of a large number of genes affecting a number of biological processes. CREB regulates a spectrum of target genes involved in cell growth regulation and differentiation, metabolism, development, neuronal activity and immune regulation (reviewed in Mayr and Montminy, 2001; Shaywitz and Greenberg, 1999). In particular CREB is thought to be an endpoint of a variety of signaling pathways critical for adaptive behaviors (for review see West et al., 2001 and Lonze et al., 2002).

Long-term changes in gene expression, thought to be the basis for long-term memory, are likely mediated in part or largely through CREB. (Bourtchuladze et al., 1994, Yin et al., 1994, See Tully et al., 2003, for review) Activation of CREB-mediated gene expression has been suggested to be a viable approach to enhancing memory in a variety of clinical settings, including neurodegenerative and psychiatric diseases (Tully et al., 2003; Jackson et al., 2003). Activation of the cAMP response or disruption of CREB activity has also been shown to affect addictive behaviors (see Chao and Nestler 2004 for review) and sensitivity to ethanol (Moore et al., 1998). Disruption of CREB activity in mice disrupts the circadian clock (Gau et al., 2002), increases anxiety and affects behavior after morphine withdrawal (Valverde et al., 2004), suggesting that modification of CREB activity might also have utility in treatment of sleep disorders, anxiety, or addiction.

CREB regulates a variety of pro-growth and anti-apoptotic genes including cfos, cyclin A, cyclinD1, PCNA, Bcl2, as well as neurotrophic factors such as BDNF. A variety of studies have also suggested that CREB activity is required for neuronal survival. Blockade of CREB and CREM in mice results in neuronal cell death and progressive neural degeneration (Mantamadiotis 2002; for review see Lonze et al 2002). Thus, activation of CREB may be neuroprotective and of clinical benefit in neurodegenerative diseases or in stroke. In addition to its role in the central nervous system, a number of metabolic regulatory genes contain CRE sites in their promoters, and the gene for PEPCK, the rate limiting enzyme in gluconeogenesis, has been shown to be regulated by cAMP. Blockade of CREB1 function in mice results in hypoglycemia (Herzig et al., 2001). CREB1 may also have a role in the immune response as CREB1 deficient mice display defective T-cell development (Rudolf et al., 1998) and ICER, an isoform of CREB1 which blocks transcription activation by CREB1, blocks IL-2 expression (Bodor et al., 2000). Activation of CREB has been suggested to be a viable approach to enhancing memory in a variety of clinical settings, including neurodegenerative and psychiatric diseases and might also be neuroprotective (Tully et al., 2003), and CREB-signaling has also been implicated in TNFα-induced vascular smooth muscle cell migration which may contribute to the formation of atherosclerotic plaques (Ono et al., 2004).

Applicants have previously disclosed that TORC1 is a potent inducer of proteins including phosphoenolpyruvate carboxy kinase (PEPCK), amphiregulin and chemokines such as IL-8 and Exodus-1/MIPalpha, and is believed to act through CREB activation (PCT Patent Publication WO 2004/085646 and Tourgenko, et al. 2003). TORC1 and other TORC proteins have been implicated in enhancing the interaction of CREB with the TAF_{II}130 component of TFIID (Conkright, et al. 2003). As such, it is believed that the TORC proteins of the present invention serve as novel drug targets for the treatment of pathological conditions related to the abnormal activation of genes that contain CRE site(s) in their promoter regions as well as for the treatment of conditions associated with abnormal activation of PEPCK, amphiregulin and chemokines, particularly IL-8 and Exodus-1/MIPalpha. These conditions include, but are not limited to, osteoarthritis, psoriasis, asthma, chronic obstructive pulmonary disease (COPD), rheumatoid arthritis, cancer, pathological angiogenesis, diabetes, hypertension, chronic pain and other inflammatory and autoimmune diseases as well as neurodegenerative conditions such as Alzheimer's Disease, Parkinson's Disease and Huntington Disease.

Thus there remains a need for modulating TORC activity. There further remains a need for identifying candidate modulators of TORC activity. In addition there remains a need to treat diseases or pathological conditions related to aberrant TORC activity in the diseased cells or tissues. The present disclosure addresses these and related needs.

### SUMMARY OF THE INVENTION

The present invention relates broadly to polynucleotide, polypeptide, and antibody compositions related to Transducer of regulated CREB (TORC) family substances, and to methods employing these compositions in research, in diagnostic applications and in therapeutic applications.

In a first aspect, a method of determining whether a biological activity of a TORC polypeptide is aberrant in a sample cell is disclosed, wherein the method includes
a) determining a first distribution of the TORC polypeptide among a plurality of subcellular fractions within the sample cell; and
b) comparing the result in a) with a second distribution of the TORC polypeptide among a plurality of subcellular fractions within one or more reference cells established as having normal distribution of TORC;
   whereby if the distribution of the TORC polypeptide in the sample cell differs from the distribution of the TORC polypeptide in the reference cells, the biological activity of the TORC polypeptide in the sample cell is aberrant. In important embodiments of this assay method, a first subcellular fraction is cytoplasmic, and in other embodiments a second subcellular fraction is nuclear. In additional embodiments the cell is an endothelial cell, or is a cell that occurs naturally in the brain.

In an additional aspect the invention includes a method to identify modulators of CREB-promoted processes in a cell that includes contacting the cell with a substance that modulates accumulation of a TORC polypeptide in a subcellular compartment of the cell. In significant embodiments of this method, the modulatory substance may be an ionophore, a stimulator of intracellular cAMP concentration, a stimulator of intracellular calcium concentration, or a stimulator of protein kinase A activity. In additional significant embodiments, the subcellular fraction may be nuclear, or an inhibitor such as an immunophilin binding agent. In further significant embodiments, the cell may be a mammalian cell, or an endothelial cell, or a cell that occurs naturally in the brain. Additionally, in this method, the cell may be cultured in vitro, or it may be ex vivo from a subject or in vivo in a subject.

In still an additional aspect the invention discloses a method of modulating CREB-promoted processes in a cell that includes contacting the cell with a substance that modulates expression of a TORC polypeptide in the cell. In important embodiments of this method, the substance may include an antisense oligonucleotide, an interfering oligonucleotide, a microoligonucleotide, a triple helix nucleic acid, a ribozyme, an RNA aptamer, double or single stranded RNA, a peptidomimetic, a polynucleotide comprising a sequence encoding a peptidomimetic, or a mixture of any two or more of them. In additional important embodiments, the cell may be a mammalian cell, or an endothelial cell, or a cell that occurs naturally in the brain. Additionally, in important embodiments of this method, the cell may be cultured in vitro, or it may be ex vivo from a subject or in vivo in a subject.

In yet a further aspect of the invention, a method is provided for substantially inhibiting the development of, treating, or ameliorating a disease or pathological condition in a subject related to an abnormal level of a CREB-promoted process in a cell. This method includes administering one or more therapeutically effective doses to the subject of a substance that modulates accumulation of a TORC polypeptide in a subcellular region of the cell. In advantageous embodiments of this therapeutic method a subcellular region may be cytoplasmic, or it may be nuclear. In additional advantageous embodiments, the pathological condition may be a neurodegenerative disease, an autoimmune disease, or an inflammatory disease, or it may be chosen from among Alzheimer's Disease, Parkinson's disease, Huntington disease, osteoarthritis, psoriasis, asthma, COPD, rheumatoid arthritis, cancer, diabetes, hypertension and chronic pain. In still further advantageous embodiments the CRE modulator alters, for example by inhibiting or enhancing, the activity or accumulation of one or more TORC proteins selected from among TORC1, TORC2 or TORC3. In yet further advantageous embodiments the TORC modulator includes one or more antibodies to a TORC protein, or fragments thereof, wherein the antibodies or fragments thereof alters the activity or accumulation of the TORC protein. In an additional advantageous embodiment, the TORC modulator includes one or more peptide mimetics to a TORC protein wherein the peptide mimic alters the activity or accumulation of the TORC protein.

In yet a further aspect of the invention, a method is provided for substantially inhibiting the development of, treating, or ameliorating a disease or pathological condition in a subject related to an abnormal level of a TORC-related process in a cell. This method includes administering one or more therapeutically effective doses to the subject of a substance that modulates accumulation of a TORC polypeptide in a subcellular region of the cell. In advantageous embodiments of this therapeutic method a subcellular region may be cytoplasmic, or it may be nuclear. In additional advantageous embodiments, the pathological condition may be a neurodegenerative disease, an autoimmune disease, or an inflammatory disease, or it may be chosen from among Alzheimer's Disease, Parkinson's disease, Huntington disease, osteoarthritis, psoriasis, asthma, COPD, rheumatoid arthritis, cancer, diabetes, hypertension and chronic pain. In still further advantageous embodiments the CRE modulator alters the accumulation of one or more TORC proteins selected from among TORC1, TORC2 or TORC3. In yet further advantageous embodiments the TORC modulator includes one or more antibodies to a TORC protein, or fragments thereof, wherein the antibodies or fragments thereof alters the accumulation of the TORC protein. In an additional advantageous embodiment, the TORC modulator includes one or more peptide mimetics to a TORC protein wherein the peptide mimic alters the accumulation of the TORC protein.

In still a further aspect the invention provides a method of substantially inhibiting the development of, treating, or ameliorating a disease or pathological condition in a subject related to an abnormal level of a TORC-activated or CREB-promoted process in a cell. This method includes administering one or more therapeutically effective doses to the subject of a substance that modulates expression of a TORC polypeptide in the cell. In significant embodiments of this therapeutic method the inhibitory substance includes an antisense oligonucleotide, an interfering oligonucleotide, a microoligonucleotide, a triple helix nucleic acid, a ribozyme, an RNA aptamer, double or single stranded RNA, a peptidomimetic, a polynucleotide comprising a sequence encoding a peptidomimetic, or a mixture of any two or more of them. In yet other significant embodiments the substance modulates the expression of any one or more TORC proteins selected from the group consisting of TORC1, TORC2 or TORC3. In yet additional significant embodiments the pathological condition may be a neurodegenerative disease, an autoimmune disease, or an inflammatory disease, or it may be chosen from among Alzheimer's Disease, Parkinson's disease, Huntington disease, osteoarthritis, psoriasis, asthma, COPD, rheumatoid arthritis, cancer, diabetes, hypertension and chronic pain.

In yet a further aspect the invention provides a method of identifying an agent that modulates the activity of a TORC-related process in a cell, including
a) introducing a TORC polypeptide into a first cell and into a reference cell;
b) contacting the first cell with the agent; and
c) determining whether a biological function of the TORC polypeptide in the first cell has been modulated in comparison with the biological function of the TORC polypeptide in the reference cell that has not been contacted with the agent;
   whereby if such a functional difference occurs the modulating agent is identified. In important embodiments of this method the biological function includes modulating the distribution of the TORC polypeptide among a plurality of subcellular compartments. In still further important embodiments a first subcellular fraction may be cytoplasmic, or a second subcellular fraction may be nuclear. In yet other important embodiments the method further includes in step b) contacting both the first cell and the reference cell with a substance that modulates accumulation of a TORC polypeptide in a nuclear fraction; and in and additional important embodiment the method includes additionally in step b) contacting both the first cell and the reference cell with a substance that modulates accumulation of a TORC polypeptide in a cytoplasmic fraction. In yet additional important embodiments a TORC polypeptide is selected from the group consisting of TORC1, TORC2 or TORC3. In still further important embodiments the first cell and the reference cell are observed in vitro or ex vivo, or are obtained from an in vivo model.

In yet an additional aspect the invention provides an antibody that binds immunospecifically to a TORC polypeptide. In an advantageous embodiment the antibody binds immunospecifically to a polypeptide whose amino acid sequence is given by SEQ ID NO:2, SEQ ID NO:4, or SEQ ID NO:6, a fragment thereof.

In still an additional aspect the invention discloses a kit for assaying, for diagnostic uses, and for research purposes, TORC-related compositions of the invention, wherein the kit includes in one or more containers, one or more components chosen from among

a) a TORC polynucleotide;

b) a TORC fusion polynucleotide;

c) a TORC polypeptide described herein;

d) a TORC fusion polypeptide; and

e) a TORC antibody.

In important embodiments of a kit including a polynucleotide, the polynucleotide may include an antisense oligonucleotide, an interfering oligonucleotide, a microoligonucleotide, a triple helix nucleic acid, a ribozyme, an RNA aptamer, double or single stranded RNA, a peptidomimetic, a polynucleotide comprising a sequence encoding a peptidomimetic, or a mixture of any two or more of them.

The invention relates broadly to various assay and diagnostic methods. In one aspect the invention discloses a method of detecting the presence of or quantifying the amount of a TORC polynucleotide in a sample that includes the steps of:
a) providing a sample comprising sample nucleic acid; and
b) detecting the presence of or quantifying the amount of a TORC polynucleotide in the sample nucleic acid.

In a significant embodiment of this method, the sample is derived from a subcellular fraction of a cell. In an additional significant embodiment a target nucleotide sequence that includes at least a portion of the TORC sequence in the sample nucleic is expanded, and furthermore the detecting or quantifying in step b) is performed on the expanded target TORC sequence. In still another significant embodiment the expanding includes reverse transcription or a polymerase chain reaction, or both. In yet other significant embodiments the detecting or quantifying in step b) includes i) fluorescence in situ hybridization of a cell or of a subcellular fraction, or ii) a real-time polymerase chain reaction. In still additional significant embodiments the detecting or quantifying in step b) includes contacting the sample nucleic acid with a probe nucleic acid that includes a TORC polynucleotide that hybridizes to a TORC polynucleotide under conditions that assure hybridization of the TORC polynucleotide to the probe, and detecting the presence of or quantifying the amount of the TORC polynucleotide that hybridizes to the probe nucleic acid. In yet further significant embodiments the TORC polynucleotide includes a label, and the detecting or quantifying includes detecting or quantifying the label. In still other significant embodiments of this method the probe nucleic acid is bound to a solid surface.

In a further aspect the invention discloses a method of identifying modulators useful in the diagnosis or prognosis of, or to developing a therapeutic strategy for, a pathology in a first subject, wherein the pathology is related to a CREB-dependent gene expression, the method including the steps of:
a) providing a sample from the first subject comprising sample nucleic acid, wherein the sample includes a TORC nucleotide sequence; and
b) determining whether the amount of the TORC sequence in the sample from the first subject differs from the amount of the TORC sequence in a reference sample, wherein the reference sample includes reference nucleic acid from a second subject known not to have the pathology;
   thus contributing to the diagnosis of, prognosis of, or developing a therapeutic strategy for, the pathology. In a significant embodiment of this method the sample is derived from a subcellular fraction of a cell. In another aspect the pathological condition may be a neurodegenerative disease, an autoimmune disease, or an inflammatory disease, or it may be chosen from among Alzheimer's Disease, Parkinson's disease, Huntington disease, osteoarthritis, psoriasis, asthma, COPD, rheumatoid arthritis, cancer, diabetes, hypertension and chronic pain. In a still further aspect, the method includes comparising gene expression profiles of genes expressed between the first sample and the reference sample.

In yet an additional aspect the invention discloses a method of detecting the presence or quantifying the amount of a TORC polypeptide in a sample including the steps of:
a) providing a sample suspected of containing a TORC polypeptide;
b) contacting the polypeptide with a specific binding agent that binds a TORC polypeptide under conditions that assure binding of the TORC polypeptide to the specific binding agent; and
c) detecting the presence or quantifying the amount of the specific binding agent that binds to the TORC polypeptide.
   In an advantageous embodiment the sample is derived from a subcellular fraction of a cell. In a further advantageous embodiment of this method the specific binding agent includes a label, or the specific binding agent binds a secondary binding agent that includes a label, and wherein the detecting or the quantifying comprises detecting or quantifying the label. In yet an additional advantageous embodiment the specific binding agent is an antibody.

In yet a further aspect the invention discloses a method of determining whether the amount of a TORC polypeptide in a sample differs from the amount of the TORC polypeptide in a reference sample, wherein the method includes the steps of:
a) providing a sample suspected to include the TORC polypeptide;
b) contacting the sample with a specific binding agent that binds a TORC polypeptide under conditions that assure binding of the TORC polypeptide to the specific binding agent; and
c) determining whether the amount of the specific binding agent that binds to the sample differs from the amount of the specific binding agent that binds to a reference under the same conditions used in step b), wherein the reference includes a standard or reference amount of the TORC polypeptide.
   In an important embodiment of this method the sample is derived from a subcellular fraction of a cell. In additional important embodiments the sample is provided from a human and the reference sample is provided from a human, or the sample is provided from a mammal and the reference is provided from the same species of mammal. In a further important embodiment of this method the specific binding agent includes a label, or the specific binding agent binds a secondary binding agent that includes a label, and wherein the detecting or the quantifying comprises detecting or quantifying the label. In yet an additional important embodiment the specific binding agent is an antibody.

In still a further aspect the invention discloses a method of contributing to the diagnosis or prognosis of, or to developing a therapeutic strategy for, a disease or pathology in a first subject, wherein the subcellular localization of a TORC polypeptide in the pathology is known to differ from the subcellular localization of the TORC polypeptide in a nonpathological state, the method comprising the steps of:
a) providing a sample from the first subject suspected to include the TORC polypeptide;
b) contacting the sample with a specific binding agent that binds a TORC polypeptide described herein under conditions that assure binding of the TORC polypeptide to the specific binding agent; and
c) determining whether the amount of the specific binding agent that binds to the sample differs from the amount of the specific binding agent that binds to a reference under the same conditions used in step b), wherein the reference is provided from a second subject known not to have the pathology;
   thus contributing to the diagnosis or prognosis of, or to developing a therapeutic strategy for, the pathology. In a significant embodiment of this method the sample is derived from a subcellular fraction of a cell. In additional significant embodiments the pathological condition may be a neurodegenerative disease, an autoimmune disease, or an inflammatory disease, or it may be chosen from among Alzheimer's Disease, Parkinson's disease, Huntington disease, osteoarthritis, psoriasis, asthma, COPD, rheumatoid arthritis, cancer, diabetes, hypertension and chronic pain. In additional significant embodiments the sample is provided from a human and the reference is provided from a human, or the sample is provided from a mammal and the reference is provided from the same species of mammal. In a further significant embodiment of this method the specific binding agent includes a label, or the specific binding agent binds a secondary binding agent that includes a label, and wherein the detecting or the quantifying comprises detecting or quantifying the label. In yet an additional important embodiment the specific binding agent is an antibody.

### BRIEF DESCRIPTION OF THE FIGURES

FIG. 1. Representation of subcellular localization of transiently transfected fluorescent TORC proteins using fluorescence microscopy. Panels A-D, HeLa cells. Panels E-H, HEK293 cells.

FIG. 2. Representation of subcellular localization of TORC2-eGFP (panel A) and TORC3-eGFP (panel B) in transfected HeLa cells as visualized by fluorescence microscopy.

FIG. 3. Representation of the results of a TORC1 translocation screen in HeLa cells. Nuclear-cytosolic fluorescence differences are plotted for each clone of the MCG full-length cDNA collection (diamond points). Leptomycin B was included in each plate as a positive control of TORC translocation (x points). The positive clones harboring TRPV6 and PKA are indicated.

FIG. 4. Representation of the localization of TORC1-eGFP in cells transfected with the empty expression vector pCMV-SPORT6 (panel A), or the expression vector containing TRPV6 (panel B) or PKA (panel C), as imaged by microscopy. The top set of panels A-C as originally filed is in color. The lower set of panels A-C was prepared by converting the color panels into grayscale images by computer.

FIG. 5. Representation of the translocation of fluorescent TORC fusion proteins in response to cAMP signaling in HeLa cells obtained by fluorescence microscopy.

FIG. 6. Representation of endogenous TORC2 translocation in HEK293 cells obtained by fluorescent immunohistochemistry (IHC) and nuclear staining (Hoechst) obtained by photomicrography.

FIG. 7. Representation of the translocation of fluorescent TORC fusion proteins in response to the GPCR agonist isoproterenol in HEK293 cells using fluorescence microscopy.

FIG. 8. Representation of the translocation of TORC1-eGFP fusion protein in HeLa cells stably expressing TORC1-eGFP in response to calcium signaling via calcineurin activation using fluorescence microscopy.

FIG. 9. Representation of the translocation of TORC1-eGFP in HeLa cells stably expressing TORC1-eGFP induced by activated calcineurin using fluorescence and photomicroscopy. The top set of panels A and B as originally filed is in color. The lower set of panels A and B was prepared by converting the color panels into grayscale images by computer.

FIG. 10. Representation of the activation of an NF-AT dependent luciferase reporter by TRPV6.

FIG. 11. Representation of the localization of TORC fusion proteins in HEK293 in response to treatment with several exogenous factors obtained using fluorescence microscopy.

FIG. 12. Representation of the translocation of TORC1-eGFP in HeLa cells in response to UV irradiation and PMA administration obtained using fluorescence microscopy.

FIG. 13. Representation of the effect of TORC1 overexpression on stimulus dependent CRE-directed transcription in HeLa cells.

FIG. 14. Representation of the requirement of TORC proteins for calcium induced CRE-driven transcription or NFKB-driven transcription in HeLa cells.

FIG. 15. Representation of western blots obtained after treating HeLa cells were either transfected with either anti-GFP specific siRNA or both TORC1 and TORC2 specific siRNA's prior to induction with various agents.

FIG. 16. Representation of the effect of TORC1(1-44)-eGFP fusion protein on promoter-driven gene expression.

FIG. 17. Representation of the translocation of fluorescent Drosophila TORC fusion protein in response to cAMP and calcium in Drosophila salivary cells, imaged by fluorescence microscopy. The top set of panels A through E as originally filed is in color. The lower set of panels A through E was prepared by converting the color panels into grayscale images by computer.

FIG. 18. TORC1 activates the transcription of PGC-1α gene mediated through CREB. HeLa cells were transfected with pGL3-basic-2 kb-PGC1α-Luc, phRL-SV40 (as control for transfection efficiency) along with various cDNA constructs as indicated. Cells were lyses and luciferase activity was determined 48 hrs post-transfection. A ratio of firefly luminescence (ff) over Renilla luminescence (RL) was calculated (mean±SEM) and expressed as normalized luciferase and used as an index of PGC-1α gene transcription.

FIG. 19. Ectopic expression of TORC1 increases the expression of PGC-1α and cytochrome c suggesting that it induces mitochondriogenesis. HeLa cells were tranduced with either the control lentiviruses (stop or GFP) or TORC1 lentivirus. A. Total protein was extracted from cells 72 hr post-transduction and subjected to Wester blot analysis to determine the expression of PGC-1α protein. B. Total RNA was extracted from cells at 24, 48 and 72 hr post-transduction and subjected to qPCR analysis to determine the expression level of endogenous PGC-1α (B) and cytochrome c (C) mRNAs.

FIG. 20. Ectopic expression of TORC1 increases cellular respiration in HeLa cells. HeLa cells were transduced with either the control (Stop) or TORC1 lentivirus. Cellular respiration was determined using a Clark electrode 72 hr post-transduction. The concentrations of Oligomycin and CCCP are 2 μg/ml and 2 μM, respectively.

FIG. 21. TORC1, 2 and 3 activate the transcription of PGC-1α gene. HeLa cells were transfected with pGL3-basic-2 kb-PGC-1α-Luc, phRL-SV40 (as control for transfection efficiency) along with various cDNA constructs as indicated. Cells were lyses and luciferase activity was determined 48 hrs post-transfection. A ratio of firefly luminescence over Renilla luminescence was calculated (mean±SEM) and expressed as Normalized luciferase activity and used as an index of PGC-1α gene transcription. N=6, *P<0.05 for TORC1, 2, 3 vs. Vector.

FIG. 22. Ectopic expression of TORC2 or TORC3 increase the expression of PGC-1α and mitochondrial markers. Primary muscle cells were tranduced with either TORC2, TORC3 or GFP adenovirus. A. Total protein was extracted from cells 48 hr post-transduction and subjected to Wester blot analysis to determine the expression of ectopic TORC2 and TORC3 protein. The primary and secondary antibodies to detect the ectopic TORC2 and TORC3 proteins were V5 and goat-anti-rabbit IgG, respectively. B-C. Total RNA was extracted from cells at 24 and 48 hr post-transduction and subjected to qPCR analysis to determine the expression level of endogenous PGC-1α (B) and PGC-1α target genes (C) including ERRα and mitochondrial markers, cytochrome C (CytC), cytochrome oxidase subunit II (CoxII), isocitrate dehydrogenase (IDH). N=3, *P<0.05 (TORC2 or TORC3 vs. GFP).

FIG. 23. Ectopic expression of TORC2 or TORC3 increase fatty acid oxidation in mouse primary muscle cells. Primary muscle cells were tranduced with either TORC2, TORC3 or GFP adenovirus. 14C-palmitate oxidation was performed 48 hr post-transduction. N=3, *P<0.05.

FIG. 24. Ectopic expression of TORC2 or TORC3 increase cellular respiration in mouse primary muscle cells. Primary muscle cells were transduced with either TORC, TORC3 or GFP adenovirus for 48 hr. Cellular respiration was measured without treatment (basal), or oligomycin or CCCP treatment. N=3, *p<0.05 TORC vs. GFP.

### DETAILED DESCRIPTION OF THE INVENTION

It is contemplated that the invention described herein is not limited to the particular methodology, protocols, and reagents described as these may vary. It is also to be understood that the terminology used herein is for the purpose of describing particular embodiments only, and is not intended to limit the scope of the present invention in any way.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices and materials are now described. All publications mentioned herein are incorporated by reference for the purpose of describing and disclosing the materials and methodologies that are reported in the publication which might be used in connection with the invention.

In practicing the present invention, many conventional techniques in molecular biology are used. These techniques are well known and are explained in, for example, Current Protocols in Molecular Biology, Volumes I, II, and III, 1997 (F. M. Ausubel ed.); Sambrook et al., 1989, Molecular Cloning: A Laboratory Manual, Second Edition, Cold Spring Harbor Laboratory Press, Cold Spring Harbor, N.Y.; DNA Cloning: A Practical Approach, Volumes I and II, 1985 (D. N. Glover ed.); Oligonucleotide Synthesis, 1984 (M. L. Gait ed.); Nucleic Acid Hybridization, 1985, (Hames and Higgins); Transcription and Translation, 1984 (Hames and Higgins eds.); Animal Cell Culture, 1986 (R. I. Freshney ed.); Immobilized Cells and Enzymes, 1986 (IRL Press); Perbal, 1984, A Practical Guide to Molecular Cloning; the series, Methods in Enzymology (Academic Press, Inc.); Gene Transfer Vectors for Mammalian Cells, 1987 (J. H. Miller and M. P. Calos eds., Cold Spring Harbor Laboratory); and Methods in Enzymology Vol. 154 and Vol. 155 (Wu and Grossman, and Wu, eds., respectively).

### ABBREVIATIONS

ATF: Activation transcription factor
cAMP: Cyclic AMP
CBP: CREB binding protein
CCE: Capacitative calcium entry
CNS: Central nervous system
COPD: Chronic obstructive pulmonary disease
CPA: cyclopiazonic acid
CRE: cAMP response element
CREB: cAMP response element binding protein
CREM: cAMP response element modulator
CsA: cyclosporin A
dTORC: Drosophila transducer of regulated CREB

### HTS: High-throughput Screening

ICER: Inducible cAMP early repressor

### LMB: Leptomycin B

NF-AT: Nuclear factor of activated T-cells
PKA: Cyclic AMP-dependent protein kinase A
SOCC: store operated calcium channel
TORC: Transducer of regulated CREB
TRPV6: Transient Receptor Potential cation channel, subfamily V, member 6

It is shown in the Examples that TORC proteins are critical co-regulators of CREB-dependent gene expression. TORC proteins were found to be present predominantly in the cytoplasm of cells but are rapidly transported to the nucleus by a variety of inducible pathways known to coordinately regulate CREB1 phosphorylation. These include agents that increase intracellular cAMP levels, agents that induce transient calcium increases, UV light, protein kinase A (PKA) activation and GPCR ligands, In addition, TORC activity is necessary and TORC nuclear-translocation sufficient for induction of CRE-mediated gene expression. Furthermore, TORC1 is translocated to the nucleus in a calcineurin dependent fashion and thus represents the second mammalian transcription factor, in addition to NF-AT, to be regulated in this fashion. Translocation of TORC1, and to a lesser extent TORC2, in response to calcium signaling is sensitive to cyclosporine A (CSA), suggesting a novel mechanism by which the efficacy and toxicity of immunophilin binding agents may occur. It is also demonstrated that the single Drosophila TORC protein, dTORC, is also regulated by calcineurin dependent nuclear translocation, further illustrating the important conserved role of TORC function. Finally, TORC translocation provides a simple biological sensor for activation of cAMP and calcium dependent signaling pathways.

The experiments reported in the Examples show that TORC translocation assays can serve in high throughput screening methods to identify modulators of TORC function and ultimately of CRE-dependent gene activation. Based on the results obtained with known modifiers it is further believed that modulators of TORC function and translocation, as well as agents that interfere with expression of a TORC polypeptide, have utility in modulating CREB1-mediated responses. In addition, it is believed that modulators of TORC function and translocation, as well as agents that interfere with expression of a TORC polypeptide, will prove effective in substantially inhibiting the development of, treating, or ameliorating a disease or pathological condition in a subject related to an abnormal level of a TORC-related or CREB-promoted process in a cell.

As used herein, the term “sample” and similar words, relate to any substance, composition or object that includes a nucleic acid, polynucleotide or oligonucleotide, or a protein or polypeptide, in a form identical to, or minimally altered from, the form of the nucleic acid, polynucleotide or oligonucleotide, or a protein or polypeptide, in an intact cell. Broadly, a sample can be a biological sample composed of intact cells. In this broad sense, DNA in a sample is genomic DNA, and RNA in a sample includes mRNA, tRNA, rRNA, and similar RNA. A sample may also contain DNA that is minimally altered from genomic DNA in view of steps such as isolating nuclei from a sample of cells, or disrupting nuclei contained in a sample of cells. In alternative meanings, a sample may be a subcellular fraction, or a subcellular component or organelle, or, when viewing an intact cell, the cell itself or a subcellular region of the cell. In several embodiments contemplated herein, a sample comprising a cell, a nucleic acid, polynucleotide or oligonucleotide, or a protein or polypeptide, is obtained from a subject suspected of having, or diagnosed as having, a particular pathology.

As used herein, the term “reference” and similar words, relate to any substance, composition or object as defined above for “sample”, with the exception that, in those embodiments of methods in which the sample is obtained from a subject suspected of having, or diagnosed as having, a particular pathology, a reference used in a cognate step in the same method is from a subject known not to have the pathology. More broadly, a reference is from a source that reliably can serve as a control, or as characterizing a nonexperimental status, or nonpathological state.

The term “agonist”, as used herein, refers to a molecule (i.e. modulator) which, directly or indirectly, may modulate a polypeptide (e.g. a TORC polypeptide) and which increase the biological activity of said polypeptide. Agonists may include proteins, nucleic acids, carbohydrates, or other molecules. A modulator that enhances gene transcription or the biochemical function of a protein is something that increases transcription or stimulates the biochemical properties or activity of said protein, respectively.

The terms “antagonist” or “inhibitor” as used herein, refer to a molecule (i.e. modulator) which directly or indirectly may modulate a polypeptide (e.g. a TORC polypeptide) which blocks or inhibits the biological activity of said polypeptide. Antagonists and inhibitors may include proteins, nucleic acids, carbohydrates, or other molecules. A modulator that inhibits expression or the biochemical function of a protein is something that reduces gene expression or biological activity of said protein, respectively.

Detection and Labeling. A TORC polynucleotide or a TORC polypeptide may be detected in many ways. Detecting may include any one or more processes that result in the ability to observe the presence and or the amount of a TORC polynucleotide or a TORC polypeptide. In one embodiment a sample nucleic acid containing a TORC polynucleotide may be detected prior to expansion. In an alternative embodiment a TORC polynucleotide in a sample may be expanded to provide an expanded TORC polynucleotide, and the expanded polynucleotide is detected or quantitated. Physical, chemical or biological methods may be used to detect and quantitate a TORC polynucleotide. Physical methods include, by way of nonlimiting example, optical visualization including various microscopic techniques such as fluorescence microscopy, confocal microscopy, microscopic visualization of in situ hybridization, surface plasmon resonance (SPR) detection such as binding a probe to a surface and using SPR to detect binding of a TORC polynucleotide or a TORC polypeptide to the immobilized probe, or having a probe in a chromatographic medium and detecting binding of a TORC polynucleotide in the chromatographic medium. Physical methods further include a gel electrophoresis or capillary electrophoresis format in which TORC polynucleotides or TORC polypeptides are resolved from other polynucleotides or polypeptides, and the resolved TORC polynucleotides or TORC polypeptides are detected. Physical methods additionally include broadly any spectroscopic method of detecting or quantitating a substance. Chemical methods include hybridization methods generally in which a TORC polynucleotide hybridizes to a probe. Biological methods include causing a TORC polynucleotide or a TORC polypeptide to exert a biological effect on a cell, and detecting the effect. The present invention discloses examples of biological effects which may be used as a biological assay. In many embodiments, the polynucleotides may be labeled as described below to assist in detection and quantitation. For example, a sample nucleic acid may be labeled by chemical or enzymatic addition of a labeled moiety such as a labeled nucleotide or a labeled oligonucleotide linker. Many equivalent methods of detecting a TORC polynucleotide or a TORC polypeptide are known to workers of skill in fields related to the field of the invention, and are contemplated to be within the scope of the invention.

A nucleic acid of the invention can be expanded using cDNA, mRNA or alternatively, genomic DNA, as a template together with appropriate oligonucleotide primers according to any of a wide range of PCR amplification techniques. The nucleic acid so amplified can be cloned into an appropriate vector and characterized by DNA sequence analysis. Furthermore, oligonucleotides corresponding to TORC nucleotide sequences can be prepared by standard synthetic techniques, e.g., using an automated DNA synthesizer.

Expanded polynucleotides may be detected and/or quantitated directly. For example, an expanded polynucleotide may be subjected to electrophoresis in a gel that resolves by size, and stained with a dye that reveals its presence and amount. Alternatively an expanded TORC polynucleotide may be detected upon exposure to a probe nucleic acid under hybridizing conditions (see below) and binding by hybridization is detected and/or quantitated. Detection is accomplished in any way that permits determining that a TORC polynucleotide has bound to the probe. This can be achieved by detecting the change in a physical property of the probe brought about by hybridizing a fragment. A nonlimiting example of such a physical detection method is SPR.

An alternative way of accomplishing detection is to use a labeled form of a TORC polynucleotide or a TORC polypeptide, and to detect the bound label. The polynucleotide may be labeled as an additional feature in the process of expanding the nucleic acid, or by other methods. A label may be incorporated into the fragments by use of modified nucleotides included in the compositions used to expand the fragment populations. A label may be a radioisotopic label, such as ¹²⁵I, ³⁵S, ³²P, ¹⁴C, or ³H, for example, that is detectable by its radioactivity. Alternatively, a label may be selected such that it can be detected using a spectroscopic method, for example. In one instance, a label may be a chromophore, absorbing incident light. A preferred label is one detectable by luminescence. Luminescence includes fluorescence, phosphorescence, and chemiluminescence. Thus a label that fluoresces, or that phosphoresces, or that induces a chemiluminscent reaction, may be employed. Examples of suitable fluorescent labels, or fluorochromes, include a ¹⁵²Eu label, a fluorescein label, a rhodamine label, a phycoerythrin label, a phycocyanin label, Cy-3, Cy-5, an allophycocyanin label, an o-phthalaldehyde label, and a fluorescamine label. Luminescent labels afford detection with high sensitivity. A label may furthermore be a magnetic resonance label, such as a stable free radical label detectable by electron paramagnetic resonance, or a nuclear label, detectable by nuclear magnetic resonance. A label may still further be a ligand in a specific ligand-receptor pair; the presence of the ligand is then detected by the secondary binding of the specific receptor, which commonly is itself labeled for detection. Nonlimiting examples of such ligand-receptor pairs include biotin and streptavidin or avidin, a hapten such as digoxigenin or antigen and its specific antibody, and so forth. A label still further may be a fusion sequence appended to a TORC polynucleotide or a TORC polypeptide. Such fusions permit isolation and/or detection and quantitation of the TORC polynucleotide or a TORC polypeptide. By way of nonlimiting example, a fusion sequence may be a FLAG sequence, a polyhistidine sequence, a fluorescent protein sequence such as a green fluorescent protein, a yellow fluorescent protein, an alkaline phosphatase, a glutathione transferase, and the like. In summary, labeling can be accomplished in a wide variety of ways known to workers of skill in fields related to the present disclosure. Any equivalent label that permits detecting and/or quantitation of a TORC polynucleotide or a TORC polypeptide is understood to fall within the scope of the invention.

Detecting, quantitating, including labeling, methods are known generally to workers of skill in fields related to the present invention, including, by way of nonlimiting example, workers of skill in spectroscopy, nucleic acid chemistry, biochemistry, molecular biology and cell biology. Quantitating permits determining the quantity, mass, or concentration of a nucleic acid or polynucleotide, or fragment thereof, that has bound to the probe. Quantitation includes determining the amount of change in a physical, chemical, or biological property as described in this and preceding paragraphs. For example the intensity of a signal originating from a label may be used to assess the quantity of the nucleic acid bound to the probe. Any equivalent process yielding a way of detecting the presence and/or the quantity, mass, or concentration of a polynucleotide or fragment thereof that hybridizes to a probe nucleic acid is envisioned to be within the scope of the present invention.

As used herein the term “substantially inhibiting the development of” a disease or pathological condition related to a TORC-related or a CREB-promoted process in a cell, and similar terms and phrases, relates to a ministration to a subject that acts prophylactically to minimize or essentially abrogate initial manifestations of the disease or condition. Such initial manifestations include, by way of nonlimiting example, a diagnostic value for a test, or a systemic symptom of the disease or condition. A subject in which or in whom the development of a disease or pathological condition has been substantially inhibited appears clinically and diagnostically indistinguishable from a normal, nondisease-bearing subject.

As used herein the term “treat” and similar terms and phrases, when employed in reference to a disease or pathological condition related to a TORC-related or a CREB-promoted process in a cell, relates to a ministration to a subject that acts to impede progression of the disease or condition or to begin an improvement in the subject, as evaluated, by way of nonlimiting example, by a diagnostic value for a test, or a systemic symptom of the disease or condition.

As used herein the term “ameliorate” and similar terms and phrases, when employed in reference to a disease or pathological condition related to a TORC-related or a CREB-promoted process in a cell, relates to a ministration to a subject that acts to reduce or essentially eliminate manifestations of the disease or condition in the subject. Such manifestations include, by way of nonlimiting example, a diagnostic value for a test, or a systemic symptom of the disease or condition.

As used herein, the term “Torc-related” or “Torc-activiated” relates to genes or proteins expressed as a result of or as a condition to Torc activity. For example, Torc-related genes or proteins inhibited or induced in pathways upstream or downstream from Torc activity or distribution.

### Polynucleotides

As used herein the terms “nucleic acid” and “polynucleotide” and similar terms and phrases are considered synonymous with each other, and are used as conventionally understood by workers of skill in fields such as biochemistry, molecular biology, genomics, and similar fields related to the field of the invention. A polynucleotide employed in the invention may be single stranded or it may be a base paired double stranded structure, or even a triple stranded base paired structure. A polynucleotide may be a DNA, an RNA, or any mixture or combination of a DNA strand and an RNA strand, such as, by way of nonlimiting example, a DNA-RNA duplex structure. A polynucleotide and an “oligonucleotide” as used herein are identical in any and all attributes defined here for a polynucleotide except for the length of a strand. As used herein, a polynucleotide may be about 50 nucleotides or base pairs in length or longer, or may be of the length of, or longer than, about 60, or about 70, or about 80, or about 100, or about 150, or about 200, or about 300, or about 400, or about 500, or about 700, or about 1000, or about 1500, or about 2000 or about 2500, or about 3000, nucleotides or base pairs or even longer. An oligonucleotide may be at least 3 nucleotides or base pairs in length, and may be shorter than about 70, or about 60, or about 50, or about 40, or about 30, or about 20, or about 15, or about 10 nucleotides or base pairs in length. Both polynucleotides and oligonucleotides may be chemically synthesized. Oligonucleotides may be used as probes.

As used herein an “isolated” nucleic acid molecule is one that is separated from at least one other nucleic acid molecule that is present in the natural source of the nucleic acid. Examples of isolated nucleic acid molecules include, but are not limited to, recombinant polynucleotide molecules, recombinant polynucleotide sequences contained in a vector, recombinant polynucleotide molecules maintained in a heterologous host cell, partially or substantially purified nucleic acid molecules, and synthetic DNA or RNA molecules. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated TORC nucleic acid molecule can contain less than about 50 kb, 25 kb, 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the present invention, e.g., a nucleic acid molecule having the nucleotide sequence of SEQ ID NO:1, SEQ ID NO:3, or SEQ ID NO:5, or a complement of any of this nucleotide sequence, can be isolated using standard molecular biology techniques and the sequence information provided herein. Using all or a portion of the nucleic acid sequence of any of SEQ ID NO:1, SEQ ID NO:3, or SEQ ID NO:5 as a hybridization probe, TORC nucleic acid sequences can be isolated using standard hybridization and cloning techniques (e.g., as described in Sambrook et al., eds., MOLECULAR CLONING: A Laboratory Manual 3rd Ed., Cold Spring Harbor Laboratory Press, Cold Spring Harbor, N.Y., 2001; and Brent et al., Current Protocols in Molecular Biology, Wiley Interscience Publishers, (2003)).

As used herein, the term “complementary” refers to Watson-Crick or Hoogsteen base pairing between nucleotides units of a nucleic acid molecule, As used herein and in the claims, the term “complementary” and similar words, relate to the ability of a first nucleic acid base in one strand of a nucleic acid, polynucleotide or oligonucleotide to interact specifically only with a particular second nucleic acid base in a second strand of a nucleic acid, polynucleotide or oligonucleotide. By way of nonlimiting example, if the naturally occurring bases are considered, A and T or U interact with each other, and G and C interact with each other. As employed in this invention and in the claims, “complementary” is intended to signify “fully complementary” within a region, namely, that when two polynucleotide strands are aligned with each other, at least in the region each base in a sequence of contiguous bases in one strand is complementary to an interacting base in a sequence of contiguous bases of the same length on the opposing strand.

As used herein, “hybridize”, “hybridization” and similar words relate to a process of forming a nucleic acid, polynucleotide, or oligonucleotide duplex by causing strands with complementary sequences to interact with each other. The interaction occurs by virtue of complementary bases on each of the strands specifically interacting to form a pair. The ability of strands to hybridize to each other depends on a variety of conditions, as set forth below. Nucleic acid strands hybridize with each other when a sufficient number of corresponding positions in each strand are occupied by nucleotides that can interact with each other. It is understood by workers of skill in the field of the present invention, including by way of nonlimiting example molecular biologists and cell biologists, that the sequences of strands forming a duplex need not be 100% complementary to each other to be specifically hybridizable.

In another embodiment, an isolated nucleic acid molecule of the invention comprises a nucleic acid molecule that is a complement of the nucleotide sequence shown in any of SEQ ID NO:1, SEQ ID NO:3, or SEQ ID NO:5, or a portion of this nucleotide sequence. A nucleic acid molecule that is complementary to the nucleotide sequence shown in any of SEQ ID NO: 1, SEQ ID NO:3, or SEQ ID NO:5 is one that is sufficiently complementary to the nucleotide sequence shown in of any of SEQ ID NO: 1, SEQ ID NO:3, or SEQ ID NO:5 that it can hydrogen bond with few or no mismatches to the nucleotide sequence shown in of any of SEQ ID NO:1, SEQ ID NO:3, or SEQ ID NO:5, thereby forming a stable duplex.

A significant use of a nucleic acid, polynucleotide, or oligonucleotide is in an assay directed to identifying a target sequence to which a probe nucleic acid hybridizes. The selectivity of a probe for a target is affected by the stringency of the hybridizing conditions. “Stringency” of hybridization reactions is readily determinable by one of ordinary skill in the art, and generally is an empirical evaluation dependent upon probe length, temperature, and buffer composition. Hybridization generally depends on the ability of denatured DNA to reanneal when complementary strands are present in an environment below their melting temperature. Higher relative temperatures tend to make the reaction conditions more stringent, while lower temperatures less so. For additional details and explanation of stringency of hybridization reactions and identifying hybridization conditions of varying stringency, see Brent et al., Current Protocols in Molecular Biology, Wiley Interscience Publishers, (2003), and Sambrook et al., Molecular Cloning: A Laboratory Manual, 3^{rd} Ed., New York: Cold Spring Harbor Press, 2001. In addition, in high throughput or multiplexed assay systems, both the probe characteristics and the stringency may be optimized to permit achieving the objectives of the multiplexed assay under a single set of stringency conditions.

Nonlimiting examples of “stringent conditions” or “high stringency conditions”, as defined herein, include those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C., or (4) employ 7% sodium dodecyl sulfate (SDS), 0.5 M NaPO₄, 1 mM EDTA at 50° C. with washing in 2×SSC, 0.1% SDS at 50° C.

“Moderately stringent conditions” include, by way of nonlimiting example, the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

### Variant TORC Polynucleotides

The invention further encompasses nucleic acid molecules that differ from the disclosed TORC nucleotide sequences. For example a sequence may differ due to degeneracy of the genetic code. These nucleic acids thus encode the same TORC protein as that encoded by the nucleotide sequence shown in SEQ ID NO:1, SEQ ID NO:3, or SEQ ID NO:5. In such embodiments, an isolated nucleic acid molecule of the invention has a nucleotide sequence encoding a protein having an amino acid sequence shown in any of SEQ ID NO:2, SEQ ID NO:4, or SEQ ID NO:6.

In addition to the human TORC nucleotide sequences shown in any of SEQ ID NO: 1, SEQ ID NO:3, or SEQ ID NO:5, it will be appreciated by those skilled in the art that DNA allelic sequence polymorphisms that lead to changes in the amino acid sequences of TORC protein may exist within a population (e.g., the human population). Such natural allelic variations can typically result in 1-5% variance in the nucleotide sequence of the TORC gene. Any and all such nucleotide variations and resulting amino acid polymorphisms in the TORC protein that are the result of natural allelic variation and that do not alter the functional activity of the TORC protein are intended to be within the scope of the invention.

Moreover, nucleic acid molecules encoding TORC orthologs from other species, and thus that have a nucleotide sequence that differs from the human sequence of any of SEQ ID NO: 1, SEQ ID NO:3, or SEQ ID NO:5, are intended to be within the scope of the invention. Nucleic acid molecules corresponding to natural allelic variants and orthologs of the TORC cDNAs of the invention can be isolated based on their homology to the human TORC nucleic acids disclosed herein using the human cDNAs, or a portion thereof, as a hybridization probe according to standard hybridization techniques under stringent hybridization conditions.

### Conservative Mutations

In addition to naturally-occurring allelic variants of the TORC sequence that may exist in the population, the skilled artisan will further appreciate that variants of the nucleotide sequence of any of SEQ ID NO: 1, SEQ ID NO:3, or SEQ ID NO:5 can be generated by a skilled artisan, thereby leading to changes in the amino acid sequence of the encoded TORC protein, without altering the functional ability of the TORC protein. For example, nucleotide substitutions leading to amino acid substitutions at “non-essential” amino acid residues can be made in the sequence of any of SEQ ID NO: 1, SEQ ID NO:3, or SEQ ID NO:5. A “non-essential” amino acid residue is a residue at a position in the sequence that can be altered from the wild-type sequence of the TORC protein without altering the biological activity of the resulting gene product, whereas an “essential” amino acid residue is a residue at a position that is required for biological activity. For example, amino acid residues that are invariant among members of a family of TORC proteins, of which the TORC proteins of the present invention are members, are predicted to be particularly unamenable to alteration. Whether a position in an amino acid sequence of a polypeptide is invariant or subject to substitution is readily apparent upon examination of a multiple sequence alignment of homologs, orthologs and paralogs of the polypeptide.

Thus an important aspect of the invention pertains to nucleic acid molecules encoding TORC proteins that contain changes in amino acid residues that are not essential for activity. Such TORC proteins differ in amino acid sequence from any of SEQ ID NO:2, SEQ ID NO:4, or SEQ ID NO:6 yet retain biological activity. In one embodiment, the isolated nucleic acid molecule comprises a nucleotide sequence encoding a protein, wherein the protein comprises an amino acid sequence at least about 75% similar to the amino acid sequence of any of SEQ ID NO:2, SEQ ID NO:4, or SEQ ID NO:6. Preferably, the protein encoded by the nucleic acid is at least about 80% identical to any of SEQ ID NO:2, SEQ ID NO:4, or SEQ ID NO:6, more preferably at least about 85%, at least about 90%, at least about 95%, at least about 97%, at least about 98%, and most preferably at least about 99% identical to SEQ ID NO:2, SEQ ID NO:4, or SEQ ID NO:6. An isolated nucleic acid molecule encoding a protein similar to the protein of any of SEQ ID NO:2, SEQ ID NO:4, or SEQ ID NO:6 can be created by introducing one or more nucleotide substitutions, additions or deletions into the corresponding nucleotide sequence, such that one or more amino acid substitutions, additions or deletions are introduced into the encoded protein.

Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. Certain amino acids have side chains with more than one classifiable characteristic, such as polar amino acid with a long aliphatic side chain. The amino acid families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, tryptophan, cysteine), nonpolar side chains (e.g., glycine, alanine, valine, leucine, isoleucine, proline, phenylalanine, methionine, tyrosine, tryptophan, lysine), beta-branched side chains (e.g., threonine, valine, isoleucine) aromatic side chains (e.g., tyrosine, phenylalanine, tryptophan, histidine) and metal-complexing side chains (e.g., aspartic acid, glutamic acid, asparagine, glutamine, serine, threonine, tyrosine, cysteine, methionine and histidine). Mutations can be introduced into SEQ ID NO:1, SEQ ID NO:3, or SEQ ID NO:5 by standard techniques, such as site-directed mutagenesis and PCR-mediated mutagenesis. Alternatively, in another embodiment, mutations can be introduced randomly along all or part of a TORC coding sequence, such as by saturation mutagenesis, and the resultant mutants can be screened for TORC protein biological activity to identify mutants that retain activity. Following mutagenesis of SEQ ID NO:1, SEQ ID NO:3, or SEQ ID NO:5 the encoded protein can be expressed by any recombinant technology known in the art and the activity of the protein can be determined.

### Determining Similarity Between Two or More Sequences

To determine the percent similarity of two amino acid sequences or of two nucleic acids, the sequences are aligned for optimal comparison purposes (e.g., gaps can be introduced in either of the sequences being compared for optimal alignment between the sequences). As used herein amino acid or nucleotide “identity” is synonymous with amino acid or nucleotide “homology”.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T or U, C, G, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region. In polypeptides the “percentage of positive residues” is calculated by comparing two optimally aligned sequences over that region of comparison, determining the number of positions at which the identical and conservative amino acid substitutions, as defined above, occur in both sequences to yield the number of matched positions, dividing the number of matched positions by the total number of positions in the region of comparison (i.e., the window size), and multiplying the result by 100 to yield the percentage of positive residues.

“Identity,” as known in the art, is a relationship between two or more polypeptide sequences or two or more polynucleotide sequences, as determined by, comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in (Computational Molecular Biology, Lesk. A. M., ed., Oxford University Press, New York, 1988; Biocomputing: Informatics and Genome Projects, Smith, D. W., ed., Academic Press, New York, 1993; Computer Analysis of Sequence Data, Part I. Griffin, A. M., and Griffin, H. G., eds. Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press. New York, 1991; and Carillo, H., and Lipman, D., SIAM J. Applied Math. (1988) 48: 1073. Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devercux, J., et al. (1984) Nucleic Acids Research 12(1): 387), BLASTP, BLASTN, and FASTA (Atschul, S. F. et al. (1990) J. Molec. Biol. 215: 403-410. The BLAST X program is publicly available from NCBI and other sources (BLAST Manual, Altschul, S., et al., NCBI NLM NIH Bethesda, Md. 20894; Altschul, S., et al. (1990) J. Mol. Biol. 215: 403-410. The well known Smith Waterman algorithm may also be used to determine identity.

Additionally the BLAST alignment tool is useful for detecting similarities and percent identity between two sequences. BLAST is available on the World Wide Web at the National Center for Biotechnology Information site. References describing BLAST analysis include Madden, T. L., Tatusov, R. L. & Zhang, J. (1996) Meth. Enzymol. 266:131-141; Altschul, S. F., Madden, T. L., Schäffer, A. A., Zhang, J., Zhang, Z., Miller, W. & Lipman, D. J. (1997) Nucleic Acids Res. 25:3389-3402; and Zhang, J. & Madden, T. L. (1997) Genome Res. 7:649-656.

### Antisense Nucleic Acids

Another aspect of the invention pertains to isolated antisense nucleic acid molecules that are hybridizable to or complementary to the nucleic acid molecule comprising the nucleotide sequence of SEQ ID NO: 1, SEQ ID NO:3, or SEQ ID NO:5, or variants, fragments, analogs or derivatives thereof. An “antisense” nucleic acid comprises a nucleotide sequence that is complementary to a “sense” nucleic acid encoding a protein, e.g., complementary to the coding strand of a double-stranded cDNA molecule or complementary to an mRNA sequence. In specific aspects, antisense nucleic acid molecules are provided that comprise a sequence complementary to a portion of at least about 10, 25, 50, 100, 250 or 500 nucleotides or an entire TORC coding strand.

In one embodiment, an antisense nucleic acid molecule is antisense to a “coding region” of the coding strand of a nucleotide sequence encoding a TORC protein. The term “coding region” refers to the region of the nucleotide sequence comprising codons which are translated into amino acid residues (e.g., the protein coding region of a human TORC protein that corresponds to any of SEQ ID NO:2, SEQ ID NO:4, or SEQ ID NO:6). In another embodiment, the antisense nucleic acid molecule is antisense to a “noncoding region” of the coding strand of a nucleotide sequence encoding a TORC protein. The term “noncoding region” refers to 5′ and 3′ sequences which flank the coding region that are not translated into amino acids (i.e., also referred to as 5′ and 3′ untranslated regions), but that may contain sequences regulating expression.

Given the coding strand sequences encoding a TORC protein disclosed herein (e.g., SEQ ID NO:1, SEQ ID NO:3, or SEQ ID NO:5), antisense nucleic acids of the invention can be designed according to the rules of Watson and Crick or Hoogsteen base pairing. The antisense nucleic acid molecule can be complementary to the entire coding region of a TORC mRNA, but more preferably is an oligonucleotide that is antisense to only a portion of the coding or noncoding region of a TORC mRNA.

The antisense nucleic acid molecules of the invention are typically administered to a subject or generated in situ such that they hybridize with or bind to cellular mRNA and/or genomic DNA encoding a TORC protein to thereby inhibit expression of the protein, e.g., by inhibiting transcription and/or translation. The hybridization can be by conventional nucleotide complementarity to form a stable duplex, or, for example, in the case of an antisense nucleic acid molecule that binds to DNA duplexes, through specific interactions in the major groove of the double helix.

### Interfering RNA

In one aspect of the invention, TORC gene expression can be attenuated by RNA interference. One approach well-known in the art is short interfering RNA (siRNA) or micro RNA (also designated as an interfering polynucleotide or a micro polynucleotide herein) mediated gene silencing where expression products of a TORC gene are targeted by specific double stranded TORC derived siRNA nucleotide sequences that are complementary to at least a 19-25 nt long segment of the TORC gene transcript, including the 5′ untranslated (UT) region, the ORF, or the 3′ UT region. See, e.g., PCT applications WO00/44895, WO99/32619, WO01/75164, WO01/92513, WO 01/29058, WO01/89304, WO02/16620, and WO02/29858, each incorporated by reference herein in their entirety; see also Jia et al. (2003) J. Virol. 77(5):3301-3306, and Morris et al. (2004) Science 305:1289-1292. Targeted genes can be a TORC gene, or an upstream or downstream modulator of the TORC gene. Nonlimiting examples of upstream or downstream modulators of a TORC gene include, e.g., a transcription factor that binds the TORC gene promoter, a kinase or phosphatase that interacts with a TORC polypeptide, and polypeptides involved in a TORC regulatory pathway.

A TORC polynucleotide according to the invention includes a siRNA polynucleotide. Such a TORC siRNA can be obtained using a TORC polynucleotide sequence, for example, by processing the TORC ribopolynucleotide sequence in a cell-free system, by transcription of recombinant double stranded TORC RNA or by chemical synthesis of nucleotide sequences similar to a TORC sequence. See, e.g., Tuschl, Zamore, Lehmann, Bartel and Sharp (1999), Genes & Dev. 13: 3191-3197, incorporated herein by reference in its entirety.

The most efficient silencing is generally observed with siRNA duplexes composed of a 21-nt sense strand and a 21-nt antisense strand, paired in a manner to have a 2-nt 3′ overhang. The sequence of the 2-nt 3′ overhang makes an additional small contribution to the specificity of siRNA target recognition. The contribution to specificity is localized to the unpaired nucleotide adjacent to the first paired bases. In one embodiment, the nucleotides in the 3′ overhang are ribonucleotides. In an alternative embodiment, the nucleotides in the 3′ overhang are deoxyribonucleotides.

In order to generate siRNA, a contemplated recombinant expression vector of the invention comprises a TORC DNA molecule cloned into an expression vector comprising operatively-linked regulatory sequences flanking the TORC sequence in a manner that allows for expression of both strands. The sense and antisense RNA strands may hybridize in vivo to generate siRNA constructs for silencing of the TORC gene by cleavage of the RNA to form siRNA molecules. Alternatively, two constructs can be utilized to create the sense and anti-sense strands of a siRNA construct. Finally, cloned DNA can encode a construct having secondary structure, wherein a single transcript has both the sense and complementary antisense sequences from the target gene or genes. In an example of this embodiment, a hairpin RNAi product is similar to all or a portion of the target gene. In another example, a hairpin RNAi product is a siRNA. The regulatory sequences flanking the TORC sequence may be identical or may be different, such that their expression may be modulated independently, or in a temporal or spatial manner.

In a specific embodiment, siRNAs are transcribed intracellularly by cloning the TORC gene templates into a vector containing, e.g., a RNA pol III transcription unit from the smaller nuclear RNA (snRNA) U6 or the human RNase P RNA H1. One example of a vector system is the GeneSuppressor RNA Interference kit (commercially available from Imgenex). The U6 and H1 promoters are members of the type III class of Pol III promoters. A siRNA vector has the advantage of providing long-term mRNA inhibition. In contrast, cells transfected with exogenous synthetic siRNAs typically recover from mRNA suppression within seven days or ten rounds of cell division. The long-term gene silencing ability of siRNA expression vectors may provide for applications in gene therapy.

In general, siRNAs are digested from longer dsRNA by an ATP-dependent ribonuclease called DICER. DICER is a member of the RNase III family of double-stranded RNA-specific endonucleases. The siRNAs assemble with cellular proteins into an endonuclease complex. In vitro studies in Drosophila suggest that the siRNAs/protein complex (siRNP) is then transferred to a second enzyme complex, called an RNA-induced silencing complex (RISC), which contains an endoribonuclease that is distinct from DICER. RISC uses the sequence encoded by the antisense siRNA strand to find and destroy mRNAs of complementary sequence. The siRNA thus acts as a guide, restricting the ribonuclease to cleave only mRNAs complementary to one of the two siRNA strands.

A TORC mRNA region to be targeted by siRNA is generally selected from a desired TORC sequence beginning 50 to 100 nt downstream of the start codon. Alternatively, 5′ or 3′ UTRs and regions nearby the start codon can be used but are generally avoided, as these may be richer in regulatory protein binding sites. UTR-binding proteins and/or translation initiation complexes may interfere with binding of the siRNP or RISC endonuclease complex. See, Elbashir et al. 2001 EMBO J. 20(23):6877-88. Hence, consideration should be taken to accommodate SNPs, polymorphisms, allelic variants or species-specific variations when targeting a desired gene.

An experiment involving a TORC siRNA includes the proper negative control. Typically, one would scramble the nucleotide sequence of the TORC siRNA and perform a homology search to make sure it lacks homology to any other gene. An inventive therapeutic method of the invention contemplates administering a TORC siRNA construct as therapy to compensate for aberrant TORC expression or activity. The TORC ribopolynucleotide is obtained and processed into siRNA fragments, or a TORC siRNA is synthesized, as described above. The TORC siRNA is administered to cells or tissues using known nucleic acid transfection techniques, as described above. A TORC siRNA specific for a TORC gene will decrease or knockdown TORC transcription products, which will lead to reduced TORC polypeptide production, resulting in reduced TORC polypeptide activity in the cells or tissues.

The present invention also encompasses a method of substantially inhibiting the development of, treating, or ameliorating a disease or pathological condition in a subject related to an abnormal level of a TORC-related or CREB-promoted process in a cell including administering to the individual an RNAi construct that targets the mRNA of the protein (the mRNA that encodes the protein) for degradation. A specific RNAi construct includes a siRNA or a double stranded gene transcript that is processed into siRNAs. Upon treatment, expression of the target TORC protein is inhibited.

### Ribozymes

The polynucleotides contemplated herein may also be ribozymes, i.e., enzymatic RNA molecules, that may be used to inhibit gene expression by catalyzing the specific cleavage of RNA. The mechanism of ribozyme action involves sequence-specific hybridization of the ribozyme molecule to complementary target RNA, followed by endonucleolytic cleavage. Examples which may be used include engineered “hammerhead” or “hairpin” motif ribozyme molecules that can be designed to specifically and efficiently catalyze endonucleolytic cleavage of gene sequences, for example, the gene for TORC1, TORC2 or TORC3. Ribozymes can be synthesized to recognize specific nucleotide sequences of a protein of interest and cleave it (Cech. J. Amer. Med. Assn. 260:3030 (1988)). Techniques for the design of such molecules for use in targeted inhibition of gene expression are well known to one of skill in fields related to the present invention.

Ribozyme methods include exposing a cell to ribozymes or inducing expression in a cell of such small RNA ribozyme molecules (Grassi and Marini, 1996, Annals of Medicine 28: 499-510; Gibson, 1996, Cancer and Metastasis Reviews 15: 287-299). Intracellular expression of hammerhead and hairpin ribozymes targeted to mRNA corresponding to at least one of the genes discussed herein can be utilized to inhibit protein encoded by the gene. Ribozymes can either be delivered directly to cells, in the form of RNA oligonucleotides incorporating ribozyme sequences, or introduced into the cell as an expression vector encoding the desired ribozymal RNA. Ribozymes can be routinely expressed in vivo in sufficient number to be catalytically effective in cleaving mRNA, and thereby modifying mRNA abundance in a cell (Cotten et al., 1989 EMBO J. 8:3861-3866).

### Aptamers

RNA aptamers can also be introduced into or expressed in a cell to modify RNA abundance or activity. RNA aptamers are specific RNA ligands for proteins, such as for Tat and Rev RNA (Good et al., 1997, Gene Therapy 4: 45-54) that can specifically inhibit their translation.

### Triple Helical Polynucleotides

Inhibition of gene expression may be achieved using “triple helix” base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) In: Huber, B. E. and B. I. Carr, Molecular and Immunologic Approaches, Futura Publishing Co., Mt. Kisco, N.Y.). These molecules may also be designed to block translation of mRNA by preventing the transcript from binding to ribosomes.

All polynucleotides, including antisense molecules, triple helix DNA, RNA aptamers and ribozymes of the present invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding the genes of the polypeptides discussed herein. Such DNA sequences may be incorporated into a wide variety of vectors with suitable RNA polymerase promoters such as T7 or SP6. Alternatively, cDNA constructs that synthesize antisense RNA constitutively or inducibly can be introduced into cell lines, cells, or tissues.

### Production of RNAs

Sense RNA (ssRNA) and antisense RNA (asRNA) of TORC are produced using known methods such as transcription in RNA expression vectors. See, e.g., Sambrook et al., Molecular Cloning, 3^{rd} Ed., Cold Spring Harbor Laboratory Press, Plainview, N.Y. (2001). siRNAs, such as 21 nt RNAs, are chemically synthesized using Expedite RNA phosphoramidites and thymidine phosphoramidite (Proligo, Germany). Synthetic oligonucleotides are deprotected and gel-purified (Elbashir et al. (2001) Genes & Dev. 15, 188-200), followed by Sep-Pak C18 cartridge (Waters, Milford, Mass., USA) purification (Tuschl et al. (1993) Biochemistry, 32:11658-11668). The RNA single strands are annealed by incubating in annealing buffer (100 mM potassium acetate, 30 mM HEPES-KOH at pH 7.4, 2 mM magnesium acetate) for 1 min at 90° C. followed by 1 h at 37° C.

PNA Moieties

In various embodiments, the TORC nucleic acids can be modified to generate peptide nucleic acids (see Hyrup et al. (1996) Bioorg Med Chem 4: 5-23). As used herein, the terms “peptide nucleic acids” or “PNAs” refer to nucleic acid mimics, e.g., DNA mimics, in which the deoxyribosephosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) above; Perry-O'Keefe et al. (1996) Proc. Natl. Acad. Sci. USA 93: 14670-675.

PNAs of the TORCs can be used in therapeutic and diagnostic applications. For example, PNAs can be used as antisense or anti-gene agents for sequence-specific modulation of gene expression by, e.g., inducing transcription or translation arrest or inhibiting replication. PNAs of the TORC proteins can also be used, e.g., in the analysis of single base pair mutations in a gene by, e.g., PNA directed PCR clamping; as artificial restriction enzymes when used in combination with other enzymes, e.g., S1 nucleases (Hyrup B. (1996) above); or as probes or primers for DNA sequence and hybridization (Hyrup et al. (1996), above; Perry-O'Keefe (1996), above).

Polypeptides

As used herein the term “protein”, “polypeptide”, or “oligopeptide”, and similar words based on these, relate to polymers of alpha amino acids joined in peptide linkage. Alpha amino acids include those encoded by triplet codons of nucleic acids, polynucleotides and oligonucleotides. They may also include amino acids with side chains that differ from those encoded by the genetic code.

As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full length gene product, encoded by the corresponding gene. Alternatively, it may be defined as the polypeptide, precursor or proprotein encoded by an open reading frame described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an open reading frame, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them. As used herein an “amino acid” designates any one of the naturally occurring alpha-amino acids that are found in proteins. In addition, the term “amino acid” designates any nonnaturally occurring amino acids known to workers of skill in protein chemistry, biochemistry, and other fields related to the present invention. These include, by way of nonlimiting example, sarcosine, hydroxyproline, norleucine, alloisoleucine, cyclohexylalanine, phenylglycine, homocysteine, dihydroxyphenylalanine, ornithine, citrulline, D-amino acid isomers of naturally occurring L-amino acids, and others. In addition an amino acid may be modified or derivatized, for example by coupling the side chain with a label. Any amino acid known to a worker of skill in the art may be incorporated into a polypeptide disclosed herein.

The term “epitope tagged” when used herein refers to a chimeric polypeptide comprising a TORC polypeptide fused to a “tag polypeptide”. The tag polypeptide has enough residues to provide an epitope against which an antibody can be made, yet is short enough such that it does not interfere with activity of the polypeptide to which it is fused. The tag polypeptide preferably also is fairly unique so that the antibody does not substantially cross-react with other epitopes. Suitable tag polypeptides generally have at least six amino acid residues and usually between about 8 and 50 amino acid residues (preferably, between about 10 and 20 amino acid residues).

As used herein, the terms “active” or “activity” and similar terms refer to form(s) of a polypeptide which retain a biological and/or an immunological activity of native or naturally-occurring TORC, wherein “biological” activity refers to a biological function (either inhibitory or stimulatory) caused by a native or naturally-occurring TORC other than the ability to induce the production of an antibody against an antigenic epitope possessed by a native or naturally-occurring TORC and an “immunological” activity refers to the ability to induce the production of an antibody against an antigenic epitope possessed by a native or naturally-occurring TORC.

TORC Proteins and Polypeptides

The TORC proteins of the invention includes an isolated TORC protein whose sequence is provided in any of SEQ ID NO:2, SEQ ID NO:4, or SEQ ID NO:6. The invention also includes a mutant or variant protein any of whose residues may be changed from the corresponding residue of SEQ ID NO:2, SEQ ID NO:4, or SEQ ID NO:6 while still encoding a protein that maintains its TORC protein-like activities and physiological functions, or a functional fragment thereof. For example, the invention includes the polypeptides encoded by the variant TORC nucleic acids described above. In the mutant or variant protein, up to 20% or more of the residues may be so changed.

In general, a TORC protein-like variant that preserves TORC protein-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a non-essential or conservative substitution as defined above. Furthermore, without limiting the scope of the invention, positions of any of SEQ ID NO:2, SEQ ID NO:4, or SEQ ID NO:6 may be substitute such that a mutant or variant protein may include one or more substitutions.

The invention also includes isolated TORC proteins, and biologically active portions thereof, or derivatives, fragments, analogs or homologs thereof. Also provided are polypeptide fragments suitable for use as immunogens to raise anti-TORC protein antibodies. A fragment of a protein or polypeptide, such as a peptide or oligopeptide, may be 5 amino acid residues or more in length, or 6 or more, 7 or more, 8 or more, 9 or more, 10 or more, 15 or more, 20 or more, 25 or more, 30 or more, 50 or more, 10 or more residues in length, up to a length that is one residue shorter than the full length sequence. In one embodiment, native TORC proteins can be isolated from cells or tissue sources by an appropriate purification scheme using standard protein purification techniques. In another embodiment, TORC proteins are produced by recombinant DNA techniques. Alternative to recombinant expression, a TORC protein or polypeptide can be synthesized chemically using standard peptide synthesis techniques. Purification of proteins and polypeptides is described, for example, in texts such as “Protein Purification, 3^{rd} Ed.”, R. K. Scopes, Springer-Verlag, New York, 1994; “Protein Methods, 2^{nd} Ed.,” D. M. Bollag, M. D. Rozycki, and S. J. Edelsterin, Wiley-Liss, New York, 1996; and “Guide to Protein Purification”, M. Deutscher, Academic Press, New York, 2001.

Biologically active portions of a TORC protein include peptides comprising amino acid sequences sufficiently similar to or derived from the amino acid sequence of the TORC protein, e.g., the amino acid sequence shown in SEQ ID NO:2, SEQ ID NO:4, or SEQ ID NO:6 that include fewer amino acids than the full length TORC proteins, and exhibit at least one activity of a TORC protein. Typically, biologically active portions comprise a domain or motif with at least one activity of the TORC protein. A biologically active portion of a TORC protein can be a polypeptide which is, for example, 10, 25, 50, 100 or more amino acids in length. A biologically active portion of a TORC protein of the present invention may contain at least one of the above-identified domains conserved among the TORC family of proteins. Moreover, other biologically active portions, in which other regions of the protein are deleted, can be prepared by recombinant techniques and evaluated for one or more of the functional activities of a native TORC protein.

In an embodiment, the TORC protein has an amino acid sequence shown in any of SEQ ID NO:2, SEQ ID NO:4, or SEQ ID NO:6. In other embodiments, the TORC protein is substantially similar to any of SEQ ID NO:2, SEQ ID NO:4, or SEQ ID NO:6 and retains the functional activity of the protein of any of SEQ ID NO:2, SEQ ID NO:4, or SEQ ID NO:6, yet differs in amino acid sequence due to natural allelic variation or mutagenesis, as described in detail below. Accordingly, in another embodiment, the TORC protein is a protein that comprises an amino acid sequence at least about 45% similar, and more preferably about 55% or more, 65% or more, 70% or more, 75% or more, 80% or more, 85% or more, 90% or more, 95% or more, 98% or more, or even 99% or more similar to the amino acid sequence of any of SEQ ID NO:2, SEQ ID NO:4, or SEQ ID NO:6 and retains the functional activity of the TORC proteins of the corresponding polypeptide having the sequence of SEQ ID NO:2, SEQ ID NO:4, or SEQ ID NO:6. Nonlimiting examples of particular amino acid residues that may changed in a variant polypeptide molecule are identified as the result of an alignment of a TORCX polypeptide with a homologous or paralogous polypeptide.

Chimeric and Fusion Proteins

The invention also provides TORC protein chimeric or fusion proteins. As used herein, a TORC protein “chimeric protein” or “fusion protein” includes a TORC polypeptide operatively linked to a non-TORC polypeptide. A “TORC polypeptide” refers to a polypeptide having an amino acid sequence corresponding to the TORC protein, whereas a “non-TORC polypeptide” refers to a polypeptide having an amino acid sequence corresponding to a protein that is not substantially similar to the TORC protein, e.g., a protein that is different from the TORC protein and that is derived from the same or a different organism. Within a fusion protein containing a TORC protein the TORC polypeptide can correspond to all or a portion of a TORC protein. In one embodiment, a TORC protein fusion protein comprises a full length TORC protein or at least one biologically active fragment of a TORC protein. In another embodiment, a TORC protein fusion protein comprises at least two fragments of a TORC protein each of which retains its biological activity. Within the fusion protein, the term “operatively linked” is intended to indicate that the TORC polypeptide and the non-TORC polypeptide are fused in-frame to each other. The non-TORC polypeptide can be fused to the N-terminus or C-terminus of the TORC polypeptide.

In another embodiment, the fusion protein is a GST-TORC protein fusion protein in which the TORC protein sequences are fused to the C-terminus of the GST (i.e., glutathione S-transferase) sequences. Such fusion proteins can facilitate the purification of recombinant TORC protein. Additional fusion embodiments include FLAG-tagged fusions and fluorescent protein fusions, useful for purification and detection of the fusion construct.

In yet another embodiment, the fusion protein is a TORC protein containing a heterologous signal sequence at its N-terminus. For example, the native TORC protein signal sequence can be removed and replaced with a signal sequence from another protein. In certain host cells (e.g., mammalian host cells), expression and/or secretion of the TORC protein can be increased through use of a heterologous signal sequence.

In another embodiment, the fusion protein is a TORC protein-immunoglobulin fusion protein in which the TORC protein sequences comprising one or more domains are fused to sequences derived from a member of the immunoglobulin protein family. The TORC protein-immunoglobulin fusion proteins of the invention can be incorporated into pharmaceutical compositions and administered to a subject to inhibit an interaction between a TORC protein ligand and a TORC protein on the surface of a cell, to thereby suppress TORC protein-mediated signal transduction in vivo.

A TORC protein chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Brent et al., Current Protocols in Molecular Biology, Wiley Interscience Publishers, (2003)). Moreover, many expression vectors are commercially available that already encode a fusion moiety (e.g., a GST polypeptide). A TORC protein-encoding nucleic acid can be cloned into such an expression vector such that the fusion moiety is linked in-frame to the TORC protein.

A “specific binding agent” of a TORC polypeptide or a TORC oligopeptide is any substance that specifically binds the TORC polypeptide or oligopeptide, but binds weakly or not at all to other polypeptides and oligopeptides. Nonlimiting examples of specific binding agents include antibodies, specific receptors for TORC polypeptides, binding domains of such antibodies and receptors, aptamers, imprinted polymers, and so forth.

TORC Agonists and Antagonists

The present invention also pertains to fragments or variants of the TORC proteins that function as either TORC protein agonists (mimetics) or as TORC protein antagonists. An agonist of the TORC protein can retain substantially the same, or a subset of, the biological activities of the naturally occurring form of the TORC protein. An antagonist of the TORC protein can inhibit one or more of the activities of the naturally occurring form of the TORC protein by, for example, competitively binding to a downstream or upstream member of a cellular signaling cascade which includes the TORC protein.

Variants or fragments of the TORC protein that function as either agonists (mimetics) or as antagonists can be identified by screening combinatorial libraries of mutants, e.g., truncation mutants, of the TORC protein for TORC protein agonist or antagonist activity. In one embodiment, a variegated library of TORC variants is generated by combinatorial mutagenesis at the nucleic acid level and is encoded by a variegated gene library. A variegated library of TORC variants can be produced by, for example, enzymatically ligating a mixture of synthetic oligonucleotides into gene sequences such that a degenerate set of potential TORC sequences is expressible as individual polypeptides, or alternatively, as a set of larger fusion proteins (e.g., for phage display) containing the set of TORC sequences therein. Chemical synthesis of a degenerate gene sequence can be performed in an automatic DNA synthesizer, and the synthetic gene then ligated into an appropriate expression vector. Methods for synthesizing degenerate oligonucleotides are known in the art (see, e.g., Narang (1983) Tetrahedron 39:3; Itakura et al. (1984) Annu Rev Biochem 53:323; Itakura et al. (1984) Science 198:1056; Ike et al. (1983) Nucl Acid Res 11:477.

As the TORC gene family contains a critical region of high conservation, peptide mimetics of TORC proteins would also be predicted to act as TORC modulators. An embodiment of a mimetic is disclosed in the Examples. Such peptides are derived or designed from TORC family proteins that block TORC function. These mimetics are expected to block function of all the highly related TORC proteins. Suitable peptide mimetics to TORC proteins can be made according to conventional methods based on an understanding of the regions in the polypeptides required for TORC protein activity. Briefly, a short amino acid sequence is identified in a protein by conventional structure-function studies such as deletion or mutation analysis of the wild-type protein, as well as by multisequence alignment. The amino acid sequence of the peptide mimetic may be composed of amino acids matching this region in whole or in part. Such amino acids could be replaced with other chemical structures resembling the original amino acids but imparting pharmacologically better properties, such as higher inhibitory activity, stability, half-life or bioavailability.

Polypeptide Libraries

In addition, libraries of fragments of the TORC protein coding sequence can be used to generate a variegated population of functional fragments for screening and subsequent selection of variants of a TORC protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of TORC proteins. Recrusive ensemble mutagenesis (REM), a new technique that enhances the frequency of functional mutants in the libraries, can be used in combination with the screening assays to identify TORC variants (Arkin and Yourvan (1992) Proc. Natl. Acad. Sci. USA 89:7811-7815; Delgrave et al. (1993) Protein Engineering 6:327-331).

### Anti-TORC Antibodies

The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. In general, antibody molecules obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species. Any antibody disclosed herein binds “immunospecifically” to its cognate antigen. By immunospecific binding is meant that an antibody raised by challenging a host with a particular immunogen binds to a molecule such as an antigen that includes the immunogenic moiety with a high affinity, and binds with only a weak affinity or not at all to non-immunogen-containing molecules. As used in this definition, high affinity means having a dissociation constant less than about 1×10⁻⁶ M, and weak affinity means having a dissociation constant higher than about 1×10⁻⁶ M.

An isolated protein of the invention intended to serve as an antigen, or a portion or fragment thereof, can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein, such as an amino acid sequence shown in SEQ ID NO:2, SEQ ID NO:4, or SEQ ID NO:6, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

### 1. Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor.

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

### 2. Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor: J. Immunol., 133:3001 (1984); Brodeur et al.: Monoclonal Antibody Production Techniques and Applications, Marcel Dekker, Inc., New York, (1987) pp. 51-63). The culture medium in which the hybridoma cells are cultured can then be assayed for the presence of monoclonal antibodies directed against the antigen. After the desired hybridoma cells are identified, the clones can be subcloned by limiting dilution procedures and grown by standard methods (Goding, 1986). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567.

### 3. Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) The humanized antibody optimally also will comprise at least a portion of an immunoglobulin constant region (Fc), typically that of a human immunoglobulin (Jones et al., 1986; Riechmann et al., 1988; and Presta (1992) Curr. Op. Struct. Biol., 2:593-596).

### 4. Human Antibodies

Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al. (1983) Immunol Today 4: 72) and the EBV hybridoma technique to produce human monoclonal antibodies (see Cole, et al. (1985) In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al. (1983) Proc Natl Acad Sci USA 80: 2026-2030) or by transforming human B-cells with Epstein Barr Virus in vitro (see Cole, et al. (1985) In: MONOCLONAL ANTIBODIES AND CANCER THERAPY, Alan R. Liss, Inc., pp. 77-96). In addition, human antibodies can also be produced using additional techniques, including phage display libraries (Hoogenboom and Winter (1991) J. Mol. Biol., 227:381; Marks et al. (1991) J. Mol. Biol., 222:581). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (1992) (Bio/Technology 10, 779-783); Lonberg et al. ((1994) Nature 368 856-859); Morrison ((1994) Nature 368, 812-13); Fishwild et al, ((1996) Nature Biotechnology 14, 845-51); Neuberger ((1996) Nature Biotechnology 14, 826); and Lonberg and Huszar ((1995) Intern. Rev. Immunol. 13 65-93). A method for producing an antibody of interest, such as a human antibody, is also disclosed in U.S. Pat. No. 5,916,771.

Human antibodies may additionally be produced using transgenic nonhuman animals which are modified so as to produce fully human antibodies rather than the animal's endogenous antibodies in response to challenge by an antigen. (See publication WO 94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications.

### 5. F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of Fab expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

### 6. Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit. Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al. (1991) EMBO J., 10:3655-3659. For further details of generating bispecific antibodies see, for example, Suresh et al. (1986) Methods in Enzymology, 121:210.

### 7. Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis(p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

### 8. Diagnostic Applications of Antibodies Directed Against TORC Proteins

Antibodies directed against a protein of the invention may be used in methods known within the art relating to the localization and/or quantitation of the protein (e.g., for use in measuring levels of the protein within appropriate physiological samples, for use in diagnostic methods, for use in imaging the protein, and the like). An anti-TORC antibody may be used to detect or quantitate a TORC protein antigen in a sample. In many such embodiments the antibody is used in an immunosorbent assay.

An antibody specific for a protein of the invention can be used to isolate the protein by standard techniques, such as immunoaffinity chromatography or immunoprecipitation. Such an antibody can facilitate the purification of the natural protein antigen from cells and of recombinantly produced antigen expressed in host cells. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S, ³H.

### 9. Pharmaceutical Compositions of Antibodies

Antibodies specifically binding a protein of the invention, as well as other molecules identified by the screening assays disclosed herein, can be administered for the treatment of various disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nanoparticles, and nanocapsules) or in macroemulsions.

### 10. Antibody Therapeutics

Antibodies of the invention, including polyclonal, monoclonal, humanized and fully human antibodies, may be used as therapeutic agents. Such agents will generally be employed to treat or prevent a disease or pathology in a subject. An antibody preparation, preferably one having high specificity and high affinity for its target antigen, is administered to the subject and will generally have an effect due to its binding with the target. Such an effect may be one of two kinds, depending on the specific nature of the interaction between the given antibody molecule and the target antigen in question. In the first instance, administration of the antibody may abrogate or inhibit the binding of the target with an endogenous ligand to which it naturally binds. In this case, the antibody binds to the target and masks a binding site of the naturally occurring ligand, wherein the ligand serves as an effector molecule. Thus the receptor mediates a signal transduction pathway for which ligand is responsible.

Alternatively, the effect may be one in which the antibody elicits a physiological result by virtue of binding to an effector binding site on the target molecule. In this case the target, a receptor having an endogenous ligand which may be absent or defective in the disease or pathology, binds the antibody as a surrogate effector ligand, initiating a receptor-based signal transduction event by the receptor.

The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

TORC Recombinant Vectors and Host Cells

Another aspect of the invention pertains to vectors, preferably expression vectors, containing a nucleic acid encoding TORC protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively linked to the nucleic acid sequence to be expressed. Within a recombinant expression vector, “operably linked” is intended to mean that the nucleotide sequence of interest is linked to a regulatory sequence(s) in a manner that allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel (1990) GENE EXPRESSION TECHNOLOGY: METHODS IN ENZYMOLOGY 185, Academic Press, San Diego, Calif. Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., TORC proteins, mutant forms of the TORC protein, fusion proteins, etc.). The recombinant expression vectors of the invention can be designed for expression of the TORC protein in prokaryotic or eukaryotic cells. For example, the TORC protein can be expressed in bacterial cells such as E. coli, insect cells (using baculovirus expression vectors) yeast cells or mammalian cells. Suitable host cells are discussed further in Goeddel (1990). Alternatively, the recombinant expression vector can be transcribed and translated in vitro, for example using T7 promoter regulatory sequences and T7 polymerase.

Promoter regions can be selected from any desired gene using vectors that contain a reporter transcription unit lacking a promoter region, such as a chloramphenicol acetyl transferase (“CAT”), or the luciferase (LUC) transcription unit, downstream of restriction site or sites for introducing a candidate promoter fragment; i.e., a fragment that may contain a promoter. For example, introduction into the vector of a promoter-containing fragment at the restriction site upstream of the CAT or LUC gene engenders production of CAT or LUC activity, respectively, which can be detected by standard CAT or LUC assays. Vectors suitable to this end are well known and readily available. Two such vectors are pKK232-8 and pCM7. Thus, promoters for expression of polynucleotides of the present invention include not only well-known and readily available promoters, but also promoters that readily may be obtained by the foregoing technique, using a reporter gene.

Expression of proteins in prokaryotes is most often carried out in E. coli with vectors containing constitutive or inducible promoters directing the expression of either fusion or non-fusion proteins. Among known bacterial promoters suitable for expression of polynucleotides and polypeptides are the E. coli lacI and lacZ promoters, the T3 and T7 promoters, the T5 tac promoter, the lambda PR, PL promoters and the trp promoter. Fusion vectors add a number of amino acids to a protein encoded therein, usually to the amino terminus of the recombinant protein. Such fusion vectors typically serve three purposes: (1) to increase expression of recombinant protein; (2) to increase the solubility of the recombinant protein; and (3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al. (1990) GENE EXPRESSION TECHNOLOGY: METHODS IN ENZYMOLOGY 185, Academic Press, San Diego, Calif. 60-89).

In another embodiment, the TORC expression vector is a yeast expression vector. Examples of vectors for expression in yeast S. cerivisae include pYepSec1 (Baldari, et al., (1987) EMBO J. 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, the TORC protein can be expressed in insect cells using baculovirus expression vectors. Baculovirus vectors available for expression of proteins in cultured insect cells (e.g., SF9 cells) include the pAc series (Smith et al. (1983) Mol Cell Biol 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed (1987) Nature 329:840) and pMT2PC (Kaufman et al. (1987) EMBO J. 6: 187-195). When used in mammalian cells, the expression vector's control functions are often provided by viral regulatory elements. For example, commonly used promoters are derived from polyoma, Adenovirus 2, cytomegalovirus and Simian Virus 40. Other eukaryotic promoters include the CMV immediate early promoter, the HSV thymidine kinase promoter, the early and late SV40 promoters, the promoters of retroviral LTRs, such as those of the Rous sarcoma virus (“RSV”), and metallothionein promoters, such as the mouse metallothionein-I promoter.

For other suitable expression systems for both prokaryotic and eukaryotic cells. See, e.g., Sambrook et al., MOLECULAR CLONING: A LABORATORY MANUAL. 3rd ed., Cold Spring Harbor Laboratory, Cold Spring Harbor Laboratory Press, Cold Spring Harbor, N.Y., 2001.

In another embodiment, the recombinant mammalian expression vector is capable of directing expression of the nucleic acid preferentially in a particular cell type. Non-limiting examples of suitable tissue-specific promoters include the albumin promoter (liver-specific; Pinkert et al. (1987) Genes Dev 1:268-277), lymphoid-specific promoters (Calame and Eaton (1988) Adv Immunol 43:235-275), in particular promoters of T cell receptors (Winoto and Baltimore (1989) EMBO J8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss (1990) Science 249:374-379) and the α-fetoprotein promoter (Campes and Tilghman (1989) Genes Dev 3:537-546).

The invention further provides a recombinant expression vector comprising a DNA molecule of the invention cloned into the expression vector in an antisense orientation. That is, the DNA molecule is operatively linked to a regulatory sequence in a manner that allows for expression (by transcription of the DNA molecule) of an RNA molecule that is antisense to a TORC mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. For a discussion of the regulation of gene expression using antisense genes see Weintraub et al., “Antisense RNA as a molecular tool for genetic analysis,” Reviews—Trends in Genetics, Vol. 1(1) 1986.

### Host Cells

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, the TORC protein can be expressed in bacterial cells such as E. coli, insect cells, yeast or mammalian cells (such as Chinese hamster ovary cells (CHO) or COS cells). Other suitable host cells are known to those skilled in the art.

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, and W138 cells.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (2001), Brent et al. (2003), and other laboratory manuals.

For stable transfection of mammalian cells, in order to identify and select stable integrants, a gene that encodes a selectable marker (e.g., resistance to antibiotics) is generally introduced into the host cells along with the gene of interest. Various selectable markers include those that confer resistance to drugs, such as G418, hygromycin and methotrexate.

### Cell Culture

A cell culture to express TORC is propagated using standard culture conditions. 24 hours before transfection, at approx. 80% confluency, the cells are trypsinized and diluted 1:5 with fresh medium without antibiotics (1-3×105 cells/ml) and transferred to 24-well plates (500 ml/well). Transfection is performed using a commercially available lipofection kit or by FuGENE6 or by electroporation, calcium phosphate particle incorporation, or ballistic particles, and TORC expression is monitored using standard techniques with positive and negative control. A positive control is cells that naturally express TORC while a negative control is cells that do not express TORC.

A host cell of the invention, such as a prokaryotic or eukaryotic host cell in culture, can be used to produce (i.e., express) the TORC protein. Accordingly, the invention further provides methods for producing the TORC protein using the host cells of the invention. In one embodiment, the method comprises culturing the host cell of invention (into which a recombinant expression vector encoding the TORC protein has been introduced) in a suitable medium such that the TORC protein is produced. In another embodiment, the method further comprises isolating the TORC protein from the medium or the host cell.

Transgenic Animals

The host cells of the invention can also be used to produce nonhuman transgenic animals. For example, in one embodiment, a host cell of the invention is a fertilized oocyte or an embryonic stem cell into which TORC protein-coding sequences have been introduced. Such host cells can then be used to create non-human transgenic animals in which exogenous TORC protein sequences have been introduced into their genome or homologous recombinant animals in which endogenous TORC protein sequences have been altered. Such animals are useful for studying the function and/or activity of the TORC proteins and for identifying and/or evaluating modulators of TORC protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous TORC gene has been altered by homologous recombination between the endogenous gene and an exogenous DNA molecule introduced into a cell of the animal, e.g., an embryonic cell of the animal, prior to development of the animal.

A transgenic animal of the invention can be created by introducing TORC protein-encoding nucleic acid into the male pronuclei of a fertilized oocyte, e.g., by microinjection, retroviral infection, and allowing the oocyte to develop in a pseudopregnant female foster animal. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan 1986, In: MANIPULATING THE MOUSE EMBRYO, Cold Spring Harbor Laboratory Press, Cold Spring Harbor, N.Y. A transgenic founder animal can then be used to breed additional animals carrying the transgene. Moreover, transgenic animals carrying a transgene encoding a TORC protein can further be bred to other transgenic animals carrying other transgenes. See e.g., Thomas et al. (1987) Cell 51:503 for a description of homologous recombination vectors. The vector is introduced into an embryonic stem cell line (e.g., by electroporation) and cells in which the introduced TORC protein gene has homologously recombined with the endogenous TORC protein gene are selected (see e.g., Li et al. (1992) Cell 69:915). See e.g., Bradley 1987, In: TERATOCARCINOMAS AND EMBRYONIC STEM CELLS: A PRACTICAL APPROACH, Robertson, ed. IRL, Oxford, pp. 113-152, and Bradley (1991) Curr Opin Biotechnol 2:823-829; PCT International Publication Nos.: WO 90/1184; WO 91/01140; WO 92/0968; and WO 93/04169.

Pharmaceutical Compositions

The pharmaceutical compositions disclosed herein useful for preventing, treating or ameliorating pathological conditions related to abnormal CRE-dependent gene expression or abnormal activation of chemokines are to be administered to a patient at therapeutically effective doses. A therapeutically effective dose refers to that amount of the compound sufficient to result in substantially inhibiting the development of, the treatment of, or the amelioration of said conditions.

Administration “in combination with” one or more further therapeutic agents includes simultaneous (concurrent) and consecutive administration in any order.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™. The TORC nucleic acid molecules, TORC proteins, and anti-TORC protein antibodies of the invention, and derivatives, fragments, analogs and homologs thereof are designated “active compounds” or “therapeutics” herein. These therapeutics can be incorporated into pharmaceutical compositions suitable for administration to a subject. Such compositions typically comprise the nucleic acid molecule, protein, or antibody and a pharmaceutically acceptable carrier.

As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in textbooks such as Remington's Pharmaceutical Sciences, Gennaro A R (Ed.) 20^{th} edition (2000) Williams & Wilkins PA, USA, and Wilson and Gisvold's Textbook of Organic Medicinal and Pharmaceutical Chemistry, by Delgado and Remers, Lippincott-Raven., which are incorporated herein by reference. Preferred examples of components that may be used in such carriers or diluents include, but are not limited to, water, saline, phosphate salts, carboxylate salts, amino acid solutions, Ringer's solutions, dextrose solution, and 5% human serum albumin. Liposomes and non-aqueous vehicles such as fixed oils may also be used.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic. For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRCN DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release pharmaceutical active agents over shorter time periods.

Microencapsulation of recombinant proteins for sustained release has been successfully performed with human growth hormone (rhGH), interferon-(rhIFN-), interleukin-2, and MN rgp120. Johnson et al., Nat. Med. 2:795-799 (1996); Yasuda, Biomed. Ther., 27:1221-1223 (1993); Hora et al., Bio/Technology, 8:755-758 (1990); Cleland, “Design and Production of Single Immunization Vaccines Using Polylactide Polyglycolide Microsphere Systems,” in Vaccine Design The Subunit and Adjuvant Approach, Powell and Newman, eds, (Plenum Press: New York. 1995), pp. 439-462; WO 97/03692, WO 96/40072, WO 96/7399; and U.S. Pat. No. 5,654,010.

The nucleic acid molecules of the invention can be inserted into vectors and used as gene therapy vectors. Gene therapy vectors may include antisense polynucleotides and inhibitory polynucleotides including microRNA (miRNA), modified miRNA, small inhibitory RNA (siRNA), and modified siRNA, wherein modifications are introduced as described in this invention at least to confer stability on the molecules. In one embodiment of gene therapy a TORC nucleic acid is part of an expression vector that expresses a TORC protein or fragment or chimeric protein thereof in a subject. In particular, such a nucleic acid has a promoter operably linked to the TORC coding region, said promoter being inducible or constitutive, and, optionally, tissue-specific. In another particular embodiment, a nucleic acid molecule is used in which the TORC coding sequences and any other desired sequences are flanked by regions that promote homologous recombination at a desired site in the genome, thus providing for intrachromosomal expression of a TORC nucleic acid (Koller and Smithies, 1989, Proc. Natl. Acad. Sci. USA 86:8932-8935; Zijlstra et al., 1989, Nature 342:435-438).

Gene therapy vectors can be delivered to a subject by any of a number of routes, e.g., as described in U.S. Pat. No. 5,703,055, by constructing it as part of an appropriate nucleic acid expression vector and administering it so that it becomes intracellular, e.g., by infection using a defective or attenuated retroviral or other viral vector (see, e.g., U.S. Pat. No. 4,980,286 and others mentioned infra), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering it in linkage to a peptide which is known to enter the nucleus, by administering it in linkage to a ligand subject to receptor-mediated endocytosis (see e.g., U.S. Pat. Nos. 5,166,320; 5,728,399; 5,874,297; and 6,030,954, all of which are incorporated by reference herein in their entirety) (which can be used to target cell types specifically expressing the receptors), etc. Delivery can thus also include, e.g., intravenous injection, local administration (see U.S. Pat. No. 5,328,470) or stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

In alternative embodiments a retroviral vector can be used (see, e.g., U.S. Pat. Nos. 5,219,740; 5,604,090; and 5,834,182). These retroviral vectors have been modified to delete retroviral sequences that are not necessary for packaging of the viral genome and integration into host cell DNA. The TORC nucleic acid to be used in gene therapy is cloned into the vector, which facilitates delivery of the gene into a patient.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Methods for conducting adenovirus-based gene therapy are described in, e.g., U.S. Pat. Nos. 5,824,544; 5,868,040; 5,871,722; 5,880,102; 5,882,877; 5,885,808; 5,932,210; 5,981,225; 5,994,106; 5,994,132; 5,994,134; 6,001,557; and 6,033,8843, all of which are incorporated by reference herein in their entirety.

Adeno-associated virus (AAV) has also been proposed for use in gene therapy. Methods for producing and utilizing AAV are described, e.g., in U.S. Pat. Nos. 5,173,414; 5,252,479; 5,552,311; 5,658,785; 5,763,416; 5,773,289; 5,843,742; 5,869,040; 5,942,496; and 5,948,675, all of which are incorporated by reference herein in their entirety.

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In a preferred embodiment, the cell used for gene therapy is autologous to the patient. Dosages and desired drug concentrations of pharmaceutical compositions of the present invention may vary depending on the particular use envisioned The determination of the appropriate dosage or route of administration is well within the skill of an ordinary physician. Animal experiments provide reliable guidance for the determination of effective doses for human therapy. Interspecies scaling of effective doses can be performed following the principles laid down by Mordenti, J. and Chappell, W. “The use of interspecies scaling in toxicokinetics” In Toxicokinetics and New Drug Development, Yacobi et al., Eds., Pergamon Press, New York 1989, pp. 42-96.

When in vivo administration of a TORC polypeptide or agonist or antagonist hereof is employed, normal dosage amounts may vary from about 10 ng/kg to up to 100 mg/kg of mammal body weight or more per day, preferably about 1 .mu.g/kg/day to 10 mg/kg/day, depending upon the route of administration. Guidance as to particular dosages and methods of delivery is provided in the literature; see, for example, U.S. Pat. Nos. 4,657,760; 5,206,344; or 5,225,212. It is anticipated that different formulations will be effective for different treatment compounds and different disorders, that administration targeting one organ or tissue, for example, may necessitate delivery in a manner different from that to another organ or tissue. Compounds and their physiologically acceptable salts and solvates may be formulated for administration by inhalation or insufflation (either through the mouth or the nose) or topical, oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound.

For buccal administration the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

Pharmaceutical compositions suitable for use in the invention include compositions wherein the active ingredients are contained in an effective amount to achieve the intended purpose. The determination of an effective dose is well within the capability of those skilled in the art.

For any compound, the therapeutically effective dose can be estimated initially either in cell culture assays, e.g., of neoplastic cells, or in animal models, usually mice, rabbits, dogs, or pigs. The animal model may also be used to determine the appropriate concentration range and route of administration. A dose may be formulated in animal models to achieve a circulating plasma concentration range that includes the IC50 (i.e., the concentration of the test compound that achieves a half-maximal inhibition of symptoms). Such information can then be used to determine useful doses and routes for administration in humans.

A therapeutically effective dose refers to that amount of active ingredient useful to prevent, treat or ameliorate a particular pathological condition of interest. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between toxic and therapeutic effects is the therapeutic index, and it can be expressed as the ratio, LD50/ED50. Pharmaceutical compositions that exhibit large therapeutic indices are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active moiety or to maintain the desired effect. Factors that may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc. Pharmaceutical formulations suitable for oral administration of proteins are described, e.g., in U.S. Pat. Nos. 5,008,114; 5,505,962; 5,641,515; 5,681,811; 5,700,486; 5,766,633; 5,792,451; 5,853,748; 5,972,387; 5,976,569; and 6,051,561.

### Additional Uses and Methods of the Invention

The nucleic acid molecules, proteins, protein homologues, and antibodies described herein can be used in one or more of the following methods: (a) screening assays; (b) predictive medicine (e.g., diagnostic assays, prognostic assays, monitoring clinical trials, and pharmacogenomics); and (c) methods of treatment (e.g., therapeutic and prophylactic).

### Screening Assays

The invention provides “screening assays” for identifying modulators, i.e., candidate or test compounds or agents (e.g., proteins, polypeptides, nucleic acids or polynucleotides, peptides, peptidomimetics, small molecules including agonists or antagonists, or other drugs) that bind to TORC proteins or have a stimulatory or inhibitory effect on, for example, TORC protein expression or TORC protein activity. Any of the assays described, as well as additional assays known to practitioners in the fields of pharmacology, hematology, internal medicine, oncology and the like, may be employed in order to screen candidate substance for their properties as therapeutic agents. As noted, the therapeutic agents of the invention encompass proteins, polypeptides, nucleic acids or polynucleotides, peptides, peptidomimetics, small molecules including agonists or antagonists, or other drugs described herein.

In one embodiment, the invention provides assays for screening candidate or test compounds which bind to or modulate the activity of a TORC protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des 12: 145).

Examples of methods for the synthesis of molecular libraries can be found in the art, for example in: DeWitt et al. (1993) Proc Natl Acad Sci U.S.A. 90:6909; Erb et al. (1994) Proc Natl Acad Sci U.S.A. 91:11422; Zuckermann et al. (1994) J Med Chem 37:2678; Cho et al. (1993) Science 261:1303; Carrell et al. (1994) Angew Chem Int Ed Engl 33:2059; Carell et al. (1994) Angew Chem Int Ed Engl 33:2061; and Gallop et al. (1994) J Med Chem 37:1233.

Libraries of compounds may be presented in solution (e.g., Houghten (1992) Biotechniques 13:412-421), or on beads (Lam (1991) Nature 354:82-84), on chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc Natl Acad Sci U.S.A. 87:6378-6382; Felici (1991) J Mol Biol 222:301-310; Ladner above.). In an important embodiment a cell-based assay includes using a transfected cell that produces a tagged TORC protein from a heterologous TORC polynucleotide. Application of a candidate active agent can be tested for effectiveness is assessed by examining the subcellular distribution of the labeled TORC (see the Examples).

In another embodiment, an assay is a cell-based assay in which a cell which expresses a membrane-bound form of a TORC protein, or a biologically active portion thereof, on the cell surface is contacted with a test compound and the ability of the test compound to bind to a TORC protein determined. The cell, for example, can of mammalian origin or a yeast cell. Determining the ability of the test compound to bind to the TORC protein can be accomplished, for example, by coupling the test compound with a radioisotope or enzymatic label such that binding of the test compound to the TORC protein or biologically active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically labeled with, for example, horseradish peroxidase, alkaline phosphatase, or luciferase, and the enzymatic label detected by determination of conversion of an appropriate substrate to product.

In another embodiment, an assay is a cell-based assay comprising contacting a cell expressing a membrane-bound form of a TORC protein, or a biologically active portion thereof, on the cell surface with a test compound and determining the ability of the test compound to modulate (e.g., stimulate or inhibit) the activity of the TORC protein or biologically active portion thereof. Determining the ability of the test compound to modulate the activity of a TORC protein or a biologically active portion thereof can be accomplished, for example, by determining the ability of the TORC protein to bind to or interact with a TORC protein target molecule. In one embodiment, a TORC protein target molecule is a component of a signal transduction pathway that facilitates transduction of an extracellular signal (e.g., a signal generated by binding of a compound to a membrane-bound TORC protein molecule) through the cell membrane and into the cell. The target, for example, can be a second intercellular protein that has catalytic activity or a protein that facilitates the association of signaling molecules with the TORC protein.

Determining the ability of the TORC protein to bind to or interact with a TORC protein target molecule can be accomplished by one of the methods described above for determining direct binding. In one embodiment, determining the ability of the TORC protein to bind to or interact with a TORC protein target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising a TORC-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the present invention is a cell-free assay comprising contacting a TORC protein or biologically active portion thereof with a test compound and determining the ability of the test compound to bind to the TORC protein or biologically active portion thereof. Binding of the test compound to the TORC protein can be determined either directly or indirectly as described above. In one embodiment, the assay comprises contacting the TORC protein or biologically active portion thereof with a known compound which binds TORC protein to form an assay mixture, contacting the assay mixture with a test compound, and determining the ability of the test compound to interact with a TORC protein, wherein determining the ability of the test compound to interact with a TORC protein comprises determining the ability of the test compound to preferentially bind to a TORC protein or biologically active portion thereof as compared to the known compound.

In another embodiment, an assay is a cell-free assay comprising contacting a TORC protein or biologically active portion thereof with a test compound and determining the ability of the test compound to modulate (e.g., stimulate or inhibit) the activity of the TORC protein or biologically active portion thereof. Determining the ability of the test compound to modulate the activity of a TORC protein can be accomplished, for example, by determining the ability of the TORC protein to bind to a TORC protein target molecule by one of the methods described above for determining direct binding. In an alternative embodiment, determining the ability of the test compound to modulate the activity of a TORC protein can be accomplished by determining the ability of the TORC protein to further modulate a TORC protein target molecule. For example, the catalytic/enzymatic activity of the target molecule on an appropriate substrate can be determined as previously described.

In yet another embodiment, the cell-free assay comprises contacting the TORC protein or biologically active portion thereof with a known compound which binds a TORC protein to form an assay mixture, contacting the assay mixture with a test compound, and determining the ability of the test compound to interact with a TORC protein, wherein determining the ability of the test compound to interact with a TORC protein comprises determining the ability of the TORC protein to preferentially bind to or modulate the activity of a TORC protein target molecule.

In more than one embodiment of the above assay methods of the present invention, it may be desirable to immobilize either a TORC protein or its target molecule to facilitate separation of complexed from uncomplexed forms of one or both of the proteins, as well as to accommodate automation of the assay. Binding of a test compound to a TORC protein, or interaction of a TORC protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix.

Other techniques for immobilizing proteins on matrices can also be used in the screening assays of the invention. For example, either the TORC protein or its target molecule can be immobilized utilizing conjugation of biotin and streptavidin. Biotinylated TORC protein or target molecules can be prepared from biotin-NHS(N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with TORC protein or target molecules, but which do not interfere with binding of the TORC protein to its target molecule, can be derivatized to the wells of the plate, and unbound target or TORC protein trapped in the wells by antibody conjugation. Methods for detecting such complexes, in addition to those described above for the GST-immobilized complexes, include immunodetection of complexes using antibodies reactive with the TORC protein or target molecule, as well as enzyme-linked assays that rely on detecting an enzymatic activity associated with the TORC protein or target molecule.

In another aspect, modulators of CREB-promoted processes are identified in a method wherein a cell is contacted with a candidate compound and the expression of a TORC mRNA or protein in the cell is determined. The level of expression of a TORC mRNA or protein in the presence of the candidate compound is compared to the level of expression of a TORC mRNA or protein in the absence of the candidate compound. The candidate compound can then be identified as a modulator of TORC expression and/or modulator of CREB-promoted processes. For example, when expression of a TORC mRNA or protein is greater (statistically significantly greater) in the presence of the candidate compound than in its absence, the candidate compound is identified as a stimulator of a TORC mRNA or protein expression. Alternatively, when expression of a TORC mRNA or protein is less (statistically significantly less) in the presence of the candidate compound than in its absence, the candidate compound is identified as an inhibitor of a TORC mRNA or protein expression. The level of a TORC mRNA or protein expression in the cells can be determined by methods described herein for detecting modulators of CREB-promoted processes or TORC mRNA or protein.

In yet another aspect of the invention, the TORC proteins can be used as “bait proteins” in a two-hybrid assay or three hybrid assay (see, e.g., U.S. Pat. No. 5,283,317; Zervos et al. (1993) Cell 72:223-232; Madura et al. (1993) J Biol Chem 268:12046-12054; Bartel et al. (1993) Biotechniques 14:920-924; Twabuchi et al. (1993) Oncogene 8:1693-1696; and Brent WO94/10300), to identify other proteins that bind to or interact with the TORC protein (“TORC protein-binding proteins” or “TORC protein-bp”) and modulate TORC protein activity. Such TORC protein-binding proteins are also likely to be involved in the propagation of signals by the TORC proteins as, for example, upstream or downstream elements of the TORC protein pathway.

The two-hybrid system is based on the modular nature of most transcription factors, which consist of separable DNA-binding and activation domains. Briefly, the assay utilizes two different DNA constructs. In one construct, the gene that codes for a TORC protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a TORC protein-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with the TORC protein. Screening can also be performed in vivo. For example, in one embodiment, the invention includes a method for screening for a modulator of activity or of latency or predisposition to a TORC protein-associated disorder by administering a test compound or to a test animal at increased risk for a TORC-associated disorder. In some embodiments, the test animal recombinantly expresses a TORC polypeptide. Activity of the polypeptide in the test animal after administering the compound is measured, and the activity of the protein in the test animal is compared to the activity of the polypeptide in a control animal not administered said polypeptide. A change in the activity of said polypeptide in said test animal relative to the control animal indicates the test compound is a modulator of latency of or predisposition to a TORC-associated disorder.

In some embodiments, the test animal is a recombinant test animal that expresses a test protein transgene or expresses the transgene under the control of a promoter at an increased level relative to a wild-type test animal. Preferably, the promoter is not the native gene promoter of the transgene.

This invention further pertains to novel agents identified by the above-described screening assays and uses thereof for treatments as described herein.

### Predictive Medicine

The present invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the present invention relates to diagnostic assays for determining modulators of CREB-promoted processes or TORC protein and/or nucleic acid expression as well as TORC protein activity, in the context of a biological sample (e.g., blood, serum, cells, tissue) to thereby determine whether an individual is afflicted with a disease or disorder, or is at risk of developing a disorder, associated with aberrant TORC expression or activity. The invention also provides for prognostic (or predictive) assays for determining whether an individual is at risk of developing a disorder associated with a TORC protein, nucleic acid expression or activity. For example, mutations in a TORC gene can be assayed in a biological sample. Such assays can be used for prognostic or predictive purpose to thereby prophylactically treat an individual prior to the onset of a disorder characterized by or associated with TORC protein, nucleic acid expression or activity.

### Diagnostic Assays

Polynucleotides or oligonucleotides corresponding to any one portion of the TORC nucleic acids of SEQ ID NO:1, SEQ ID NO:3, or SEQ ID NO:5 may be used to detect DNA containing a corresponding ORF gene, or detect the expression of a corresponding TORC gene, or TORC protein-like gene. For example, a TORC nucleic acid expressed in a particular cell or tissue can be used to identify the presence of that particular cell type.

An exemplary method for detecting the presence or absence of determining modulators of CREB-promoted processes or TORC in a biological sample involves obtaining a biological sample from a test subject and contacting the biological sample with a compound or an agent capable of detecting a TORC protein or nucleic acid (e.g., mRNA, genomic DNA) that encodes a TORC protein such that the presence of a TORC is detected in the biological sample. An agent for detecting a TORC mRNA or genomic DNA is a labeled nucleic acid probe capable of hybridizing to a TORC mRNA or genomic DNA. The nucleic acid probe can be, for example, a full-length TORC nucleic acid, such as the nucleic acid of SEQ ID NO:1, SEQ ID NO:3, or SEQ ID NO:5, or a portion thereof, such as an oligonucleotide of at least 15, 30, 50, 100, 250 or 500 nucleotides in length and sufficient to specifically hybridize under stringent conditions to a TORC mRNA or genomic DNA, as described above. Other suitable probes for use in the diagnostic assays of the invention are described herein.

An agent for detecting a TORC protein is an antibody capable of binding to a TORC protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect a TORC mRNA, protein, or genomic DNA in a biological sample in vitro as well as in vivo. For example, in vitro techniques for detection of a TORC mRNA include Northern hybridizations and in situ hybridizations. In vitro techniques for detection of a TORC protein include enzyme linked immunosorbent assays (ELISAs), Western blots, immunoprecipitations and immunofluorescence. In vitro techniques for detection of a TORC genomic DNA include Southern hybridizations. Furthermore, in vivo techniques for detection of a TORC protein include introducing into a subject a labeled anti-TORC protein antibody. For example, the antibody can be labeled with a radioactive marker whose presence and location in a subject can be detected by standard imaging techniques.

The invention also encompasses kits for detecting the presence of determining modulators of CREB-promoted processes or TORC in a biological sample. For example, the kit can comprise: a labeled compound or agent capable of detecting determining modulators of CREB-promoted processes, TORC protein or mRNA in a biological sample; means for determining the amount of a TORC in the sample; and means for comparing the amount of a TORC in the sample with a standard. The compound or agent can be packaged in a suitable container. The kit can further comprise instructions for using the kit to detect a TORC protein or nucleic acid.

### Prognostic Assays

The diagnostic methods described herein can furthermore be utilized to identify subjects having or at risk of developing a disease or disorder associated with aberrant TORC expression or activity. For example, the assays described herein, such as the preceding diagnostic assays or the following assays, can be utilized to identify a subject having or at risk of developing a disorder associated with a TORC protein, nucleic acid expression or activity. Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant TORC expression or activity. For example, such methods can be used to determine whether a subject can be effectively treated with an agent for a disorder, such as a proliferative disorder, differentiative disorder, glia-associated disorders, etc. Thus, the present invention provides methods for determining whether a subject can be effectively treated with an agent for a disorder associated with aberrant TORC expression or activity in which a test sample is obtained and a TORC protein or nucleic acid is detected (e.g., wherein the presence of a TORC protein or nucleic acid is diagnostic for a subject that can be administered the agent to treat a disorder associated with aberrant TORC expression or activity.)

The methods of the invention can also be used to detect genetic lesions in a TORC gene or genes which modulate TORC expression and activity, thereby determining if a subject with the lesioned gene is at risk for, or suffers from, a proliferative disorder, differentiative disorder, glia-associated disorder, etc. In various embodiments, the methods include detecting, in a sample of cells from the subject, the presence or absence of a genetic lesion characterized by at least one of an alteration affecting the integrity of a gene encoding a TORC protein, or the misexpression of the TORC gene.

### Monitoring Clinical Efficacy

Monitoring the effect of agents (e.g., drugs, compounds) on the expression or activity of a TORC can be applied in clinical trials. For example, the effectiveness of an agent determined by a screening assay as described herein to increase TORC gene expression, protein levels, or promote the biological function of TORC, can be monitored in clinical trials of subjects exhibiting decreased TORC gene expression, protein levels, or downregulated TORC activity. Alternatively, the effectiveness of an agent determined by a screening assay to decrease TORC gene expression, protein levels, or inhibit the biological function of TORC, can be monitored in clinical trials of subjects exhibiting increased TORC gene expression, protein levels, or upregulated TORC activity. In such clinical trials, the expression or activity of a TORC and, preferably, other genes that have been implicated in, for example, a proliferative or neurological disorder, can be used as a “read out” or marker of the responsiveness of a particular cell. Such methods include assessing TORC subcellular distribution, or assaying the CREB co-activating activity of TORC.

### Methods of Treatment

The present invention provides for both prophylactic and therapeutic methods of treating a subject at risk of (or susceptible to) a disorder or having a disorder associated with aberrant TORC expression or activity.

Diseases and disorders that are characterized by increased (relative to a subject not suffering from the disease or disorder) levels or biological activity may be treated with therapeutics that antagonize (i.e., reduce or inhibit) activity. Therapeutics that antagonize activity may be administered in a therapeutic or prophylactic manner. Therapeutics that may be utilized include, but are not limited to, (i) a TORC polypeptide, or analogs, derivatives, fragments or homologs thereof, (ii) antibodies to a TORC peptide; (iii) nucleic acids encoding a TORC peptide; (iv) administration of antisense or siRNA TORC nucleic acid; or (v) modulators (i.e., inhibitors, agonists and antagonists, including additional peptide mimetic of the invention or antibodies specific to a peptide of the invention) that alter the interaction between a TORC peptide and its binding partner.

Diseases and disorders that are characterized by decreased (relative to a subject not suffering from the disease or disorder) levels or biological activity may be treated with therapeutics that increase (i.e., are agonists to) activity. Therapeutics that upregulate activity may be administered in a therapeutic or prophylactic manner. Therapeutics that may be utilized include, but are not limited to, a TORC peptide, or analogs, derivatives, fragments or homologs thereof, or an agonist that increases bioavailability.

In one aspect, the invention provides a method for preventing, in a subject, a disease or condition associated with aberrant TORC expression or activity, by administering to the subject an agent that modulates TORC expression or at least one TORC activity. Subjects at risk for a disease that is caused or contributed to by aberrant TORC expression or activity can be identified by, for example, any or a combination of diagnostic or prognostic assays as described herein. Administration of a prophylactic agent can occur prior to the manifestation of symptoms characteristic of the TORC aberrancy, such that a disease or disorder is prevented or, alternatively, delayed in its progression. Depending on the type of a TORC aberrancy, for example, a TORC agonist or TORC antagonist agent can be used for treating the subject. The appropriate agent can be determined based on screening assays described herein.

Another aspect of the invention pertains to methods of modulating TORC expression or activity for therapeutic purposes. The modulatory method of the invention involves contacting a cell with an agent that modulates one or more of the activities of a TORC protein activity associated with the cell. An agent that modulates a TORC protein activity can be an agent as described herein, such as a nucleic acid or a protein, a naturally-occurring cognate ligand of a TORC protein, a peptide, a TORC peptidomimetic, or other small molecule.

TORC Polynucleotides and Polypeptides

The present invention discloses TORC polynucleotides and the polypeptides encoded by them. TORC polynucleotides may be isolated, characterized and prepared as described in Tourgenko e al. (2003), and in U.S. Provisional application Ser. No. ______ filed (month, day, year). A polynucleotide encoding human TORC1 (GenBank Acc. No. AY360171) is shown in Table 1.

TABLE 1

(SEQ ID NO:1)

| | |
|---|---|
| 1 | aggaggagga ggtggcggcg agaagatggc gacttcgaac aatccgcgga aattcagcga |
| 61 | gaagatcgcg ctgcacaatc agaagcaggc ggaggagacg gcggccttcg aggaggtcat |
| 121 | gaaggacctg agcctgacgc gggccgcgcg gctccagctc cagaaatccc agtacctgca |
| 181 | actgggcccc agccgaggcc agtactatgg cgggtccctg cccaacgtga accagatcgg |
| 241 | gagtggcacc atggacctgc ccttccagcc cagcggattt ctgggggagg ccctggcagc |
| 301 | ggctcctgtc tctctgaccc ccttccaatc ctcgggcctg gacaccagcc ggaccacccg |
| 361 | gcaccatggg ctggtggaca gggtgtaccg ggagcgtggc cggctcggct ccccacaccg |
| 421 | ccggcccctg tcagtggaca aacacggacg gcaggccgac agctgcccct atggcaccat |
| 481 | gtacctctca ccacccgcgg acaccagctg gagaaggacc aattctgact ccgccctgca |
| 541 | ccagagcaca atgacgccca cgcagccaga atcctttagc agtgggtccc aggacgtgca |
| 601 | ccagaaaaga gtcttactgt taacagtccc aggaatggaa gagaccacat cagaggcaga |
| 661 | caaaaacctt tccaagcaag catgggacac caagaagacg gggtccaggc ccaagtcctg |
| 721 | tgaggtcccc ggaatcaaca tcttcccgtc tgccgaccag gaaaacacta cagccctgat |
| 781 | ccccgccacc cacaacacag gggggtccct gcccgacctg accaacatcc acttcccctc |
| 841 | cccgctcccg accccgctgg accccgagga gcccaccttc cctgcactga gcagctccag |
| 901 | cagcaccggc aacctcgcgg ccaacctgac gcacctgggc atcggtggcg ccggccaggg |
| 961 | aatgagcaca cctggctcct ctccacagca ccgcccagct ggcgtcagcc ccctgtccct |
| 1021 | gagcacagag gcaaggcgtc agcaggcatc gcccaccctg tccccgctgt cacccatcac |
| 1081 | tcaggctgta gccatggacg ccctgtctct ggagcagcag ctgccctacg ccttcttcac |
| 1141 | ccaggcgggc tcccagcagc caccgccgca gccccagccc ccgccgcctc ctccacccgc |
| 1201 | gtcccagcag ccaccacccc cgccaccccc acaggcgccc gtccgcctgc cccctggtgg |
| 1261 | ccccctgttg cccagcgcca gcctgactcg tgggccacag ccgcccccgc ttgcagtcac |
| 1321 | ggtaccgtcc tctctccccc agtccccccc agagaaccct ggccagccat cgatggggat |
| 1381 | cgacatcgcc tcggcgccgg ctctgcagca gtaccgcact agcgccggct ccccggccaa |
| 1441 | ccagtctccc acctcgccag tctccaatca aggcttctcc ccagggagct ccccgcaaca |
| 1501 | cacttccacc ctgggcagcg tgtttgggga cgcgtactat gagcagcaga tggcggccag |
| 1561 | gcaggccaat gctctgtccc accagctgga gcagttcaac atgatggaga acgccatcag |
| 1621 | ctccagcagc ctgtacagcc cgggctccac actcaactac tcgcaggcgg ccatgatggg |
| 1681 | cctcacgggc agccacggga gcctgccgga ctcgcagcaa ctgggatacg ccagccacag |
| 1741 | tggcatcccc aacatcatcc tcacagtgac aggagagtcc ccccccagcc tctctaaaga |
| 1801 | actgaccagc tctctggccg gggtcggcga cgtcagcttc gactccgaca gccagtttcc |
| 1861 | cctggacgaa ctcaagatcg accccctgac cctcgacgga ctgcacatgc tcaacgaccc |
| 1921 | cgacatggtt ctggccgacc cagccaccga ggacaccttc cggatggacc gcctgtgagc |
| 1981 | gggcacgccg gcaccctgcc gctcagccgt cccgacggcg cctccccagc ccggggacgg |
| 2041 | ccgtgctccg tccctcgcca acggccgagc ttgtgattct gagcttgcaa tgccgccaag |
| 2101 | cgccccccgc cagcccgccc ccggttgtcc acctcccgcg aagcccaatc gcgaggccgc |
| 2161 | gagccgggcc gtccacccac ccgcccgccc agggctgggc tgggatcgga ggccgtgagc |
| 2221 | ctcccgcccc tgcagaccct ccctgcactg gctccctcgc ccccagcccc ggggcctgag |
| 2281 | ccgtcccctg taagatgcgg gaagtgtcag ctcccggcgt ggcgggcagg ctcaggggag |
| 2341 | gggcgcgcat ggtccgccag ggctgtgggc cgtggcgcat tttccgactg tttgtccagc |
| 2401 | tctcactgcc ttccttggtt cccggtcccc cagcccatcc gccatcccca gcccgtggtc |
| 2461 | aggtagagag tgagccccac gccgccccag ggaggaggcg ccagagcgcg gggcagacgc |
| 2521 | aaagtgaaat aaacactatt ttgacggcaa aaaaaaaaaa aaa |

In the sequence shown in Table 1, the coding sequence extends from position 26 to position 1978.

The polypeptide sequence predicted for the TORC1 protein based on the nucleotide sequence of Table 1 is shown in Table 2 (GenBank Acc. No. AAQ98856.1).

TABLE 2

(SEQ ID NO:2)

| | |
|---|---|
| 1 | matsnnprkf sekialhnqk qaeetaafee vmkdlsltra arlqlqksqy lqlgpsrgqy |
| 61 | yggslpnvnq igsgtmdlpf qpsgflgeal aaapvsltpf qssgldtsrt trhhglvdrv |
| 121 | yrergrlgsp hrrplsvdkh grqadscpyg tmylsppadt swrrtnsdsa lhqstmtptq |
| 181 | pesfssgsqd vhqkrvlllt vpgmeettse adknlskqaw dtkktgsrpk scevpginif |
| 241 | psadqentta lipathntgg slpdltnihf psplptpldp eeptfpalss ssstgnlaan |
| 301 | lthlgiggag qgmstpgssp qhrpagvspl slstearrqq asptlsplsp itqavamdal |
| 361 | sleqqlpyaf ftqagsqqpp pqpqpppppp pasqqppppp ppqapvrlpp ggpllpsasl |
| 421 | trgpqpppla vtvpsslpqs ppenpgqpsm gidiasapal qqyrtsagsp anqsptspvs |
| 481 | nqgfspgssp qhtstlgsvf gdayyeqqma arqanalshq leqfnmmena isssslyspg |
| 541 | stlnysqaam mgltgshgsl pdsqqlgyas hsgipniilt vtgesppsls keltsslagv |
| 601 | gdvsfdsdsq fpldelkidp ltldglhmln dpdmvladpa tedtfrmdrl |

A polynucleotide encoding human TORC2 (GenBank Acc. No. AY360172) is shown in Table 3.

TABLE 3

(SEQ ID NO:3)

| | |
|---|---|
| 1 | atctaggctg gggccgggtt cgcggtgctc gctgaggcgg cggtggctac ggctggagga |
| 61 | gccgggccga ggccgcggcg gaggccgcgg ctggtactgg gagggtggca gggagggacg |
| 121 | gggaaggaag atggcgacgt cgggggcgaa cgggcctggt tcggccacgg cctcggcttc |
| 181 | caatccgcgc aaatttagtg agaagattgc gctgcagaag cagcgtcagg ccgaggagac |
| 241 | ggcggccttc gaggaggtga tgatggacat cggctccacc cggttacagg cccaaaaact |
| 301 | gcgactggca tacacaagga gctctcatta tggtgggtct ctgcccaatg ttaaccagat |
| 361 | tggctctggc ctggccgagt tccagagccc cctccactca cctttggatt catctcggag |
| 421 | cactcggcac catgggctgg tggaacgggt gcagcgagat cctcgaagaa tggtgtcccc |
| 481 | acttcgccga tacacccgcc acattgacag ctctccctat agtcctgcct acttatctcc |
| 541 | tcccccagag tctagctggc gaaggacgat ggcctggggc aatttccctg cagagaaggg |
| 601 | gcagttgttt cgactaccat ctgcacttaa caggacaagc tctgactctg cccttcatac |
| 661 | aagtgtgatg aaccccagtc cccaggatac ctacccaggc cccacacctc ccagcatcct |
| 721 | gcccagccga cgtgggggta ttctggatgg tgaaatggac cccaaagtac ctgctattga |
| 781 | ggagaacttg ctagatgaca agcatttgct gaagccatgg gatgctaaga agctatcctc |
| 841 | atcctcttcc cgacctcggt cctgtgaagt ccctggaatt aacatctttc catctcctga |
| 901 | ccagcctgcc aatgtgcctg tcctcccacc tgccatgaac acggggggct ccctacctga |
| 961 | cctcaccaac ctgcactttc ccccaccact gcccaccccc ctggaccctg aagagacagc |
| 1021 | ctaccctagc ctgagtgggg gcaacagtac ctccaatttg acccacacca tgactcacct |
| 1081 | gggcatcagc agggggcatg ggcctgggcc cggctatgat gcaccaggac ttcattcacc |
| 1141 | tctcagccac ccatccctgc agtcctccct aagcaatccc aacctccagg cttccctgag |
| 1201 | cagtcctcag ccccagcttc agggctccca cagccacccc tctctgcctg cctcctcctt |
| 1261 | ggcctgccat gtactgccca ccacctccct gggccacccc tcactcagtg ctccggctct |
| 1321 | ctcctcctcc tcttcctcct cctccacttc atctcctgtt ttgggcgccc cctcttaccc |
| 1381 | tgcttctacc cctggggcct ccccccacca ccgccgtgtg cccctcagcc ccctgagttt |
| 1441 | gctcgcgggc ccagccgacg ccagaaggtc ccaacagcag ctgcccaaac agttttcgcc |
| 1501 | aacaatgtca cccaccttgt cttccatcac tcagggcgtc cccctggata ccagtaaact |
| 1561 | gtccactgac cagcggttac ccccctaccc atacagctcc ccaagtctgg ttctgcctac |
| 1621 | ccagccccac accccaaagt ctctacagca gccagggctg ccctctcagt cttgttcagt |
| 1681 | gcagtcctca ggtgggcagc ccccaggcag gcagtctcat tatgggacac cgtacccacc |
| 1741 | tgggcccagt gggcatgggc aacagtctta ccaccggcca atgagtgact tcaacctggg |
| 1801 | gaatctggag cagttcagca tggagagccc atcagccagc ctggtgctgg atccccctgg |
| 1861 | cttttctgaa gggcctggat ttttaggggg tgaggggcca atgggtggcc cccaggatcc |
| 1921 | ccacaccttc aaccaccaga acttgaccca ctgttcccgc catggctcag ggcctaacat |
| 1981 | catcctcaca ggggactcct ctccaggttt ctctaaggag attgcagcag ccctggccgg |
| 2041 | agtgcctggc tttgaggtgt cagcagctgg attggagcta gggcttgggc tagaagatga |
| 2101 | gctgcgcatg gagccactgg gcctggaagg gctaaacatg ctgagtgacc cctgtgccct |
| 2161 | gctgcctgat cctgctgtgg aggagtcatt ccgcagtgac cggctccaat gagggcacct |
| 2221 | catcaccatc cctcttcttg gccccatccc ccaccaccat tcctttcctc ccttccccct |
| 2281 | ggcaggtaga gactctactc tctgtcccca gatcctcttt ctagcatgaa tgaaggatgc |
| 2341 | caagaatgag aaaaagcaa |

In the sequence shown in Table 3, the coding sequence extends from position 131 to position 2212.

The polypeptide sequence predicted for the TORC2 protein based on the nucleotide sequence of Table 2 is shown in Table 4 (GenBank Acc. No. AAQ98857.1).

TABLE 4

(SEQ ID NO:4)

| | |
|---|---|
| 1 | matsgangpg satasasnpr kfsekialqk qrqaeetaaf eevmmdigst rlqaqklrla |
| 61 | ytrsshyggs lpnvnqigsg laefqsplhs pldssrstrh hglvervqrd prrmvsplrr |
| 121 | ytrhidsspy spaylspppe sswrrtmawg nfpaekgqlf rlpsalnrts sdsalhtsvm |
| 181 | npspqdtypg ptppsilpsr rggildgemd pkvpaieenl lddkhllkpw dakklsssss |
| 241 | rprscevpgi nifpspdqpa nvpvlppamn tggslpdltn lhfppplptp ldpeetayps |
| 301 | lsggnstsnl thtmthlgis rghgpgpgyd apglhsplsh pslqsslsnp nlqaslsspq |
| 361 | pqlqgshshp slpasslach vlpttslghp slsapalsss ssssstsspv lgapsypast |
| 421 | pgasphhrrv plsplsllag padarrsqqq lpkqfsptms ptlssitqgv pldtsklstd |
| 481 | qrlppypyss pslvlptqph tpkslqqpgl psqscsvqss ggqppgrqsh ygtpyppgps |
| 541 | ghgqqsyhrp msdfnlgnle qfsmespsas lvldppgfse gpgflggegp mggpqdphtf |
| 601 | nhqnlthcsr hgsgpniilt gdsspgfske iaaalagvpg fevsaaglel glgledelrm |
| 661 | eplgleglnm lsdpcallpd paveesfrsd rlq |

A polynucleotide encoding human TORC3 (GenBank Acc. No. AY360173) is shown in Table 5.

TABLE 5

(SEQ ID NO:5)

| | |
|---|---|
| 1 | attcgccatg gccgcctcgc cgggctcggg cagcgccaac ccgcggaagt tcagtgagaa |
| 61 | gatcgcgctg cacacgcaga gacaggccga ggagacgcgg gccttcgagc agctcatgac |
| 121 | cgacctcacc ctgtcgcggg ttcaatttca gaagcttcag caactgcgcc ttacacagta |
| 181 | ccatggagga tccttaccaa atgtgagcca gctgcggagc aatgcgtcag agtttcagcc |
| 241 | gtcatttcac caagctgata atgttcgggg aacccgccat cacgggctgg tggagaggcc |
| 301 | atccaggaac cgcttccacc ccctccaccg aaggtctggg gacaagccag ggcgacaatt |
| 361 | tgatggtagt gcttttggag ccaattattc ctcacagcct ctggatgaga gttggccaag |
| 421 | gcagcagcct ccttggaaag acgaaaagca tcctgggttc aggctgacat ctgcacttaa |
| 481 | caggaccaat tctgattctg ctcttcacac gagtgctctg agtaccaagc cccaggaccc |
| 541 | ctatggagga gggggccagt cggcctggcc tgccccatac atggggtttt gtgatggtga |
| 601 | gaataatgga catggggaag tagcatcttt ccctggccca ttgaaagaag agaatctgtt |
| 661 | aaatgttcct aagccactgc caaaacaact gtgggagacc aaggagattc agtccctgtc |
| 721 | aggacgccct cgatcctgtg atgttggagg tggcaatgct tttccacata atggtcaaaa |
| 781 | cctaggcctc tcacccttct tggggacttt gaacactgga gggtcattgc cagatctaac |
| 841 | caacctccac tactcgacac ccctgccagc ctccctggac accaccgacc accactttgg |
| 901 | cagtatgagt gtggggaata gtgtgaacaa catcccagct gctatgaccc acctgggtat |
| 961 | aagaagctcc tctggtctcc agagttctcg gagtaacccc tccatccaag ccacgctcaa |
| 1021 | taagactgtg ctttcctctt ccttaaataa ccacccacag acatctgttc ccaacgcatc |
| 1081 | tgctcttcac ccttcgctcc gtctgttttc ccttagcaac ccatctcttt ccaccacaaa |
| 1141 | cctgagcggc ccgtctcgcc gtcggcagcc tcccgtcagc cctctcacgc tttctcctgg |
| 1201 | ccctgaagca catcaaggtt tcagcagaca gctgtcttca accagcccac tggccccata |
| 1261 | tcctacctcc cagatggtgt cctcagaccg aagccaactt tcctttctgc ccacagaagc |
| 1321 | tcaagcccag gtgtcgccgc caccccctta ccctgcaccc caggagctca cccagcccct |
| 1381 | cctgcagcag ccccgcgccc ctgaggcccc tgcccagcag ccccaggcag cctcctcact |
| 1441 | gccacagtca gactttcagc ttctcccggc ccagggctca tctttgacca acttcttccc |
| 1501 | agatgtgggt tttgaccagc agtccatgag gccaggccct gcctttcctc aacaggtgcc |
| 1561 | tctggtgcaa caaggttccc gagaactgca ggactctttt catttgagac caagcccgta |
| 1621 | ttccaactgc gggagtctcc cgaacaccat cctgccagaa gactccagca ccagcctgtt |
| 1681 | caaagacctc aacagtgcgc tggcaggcct gcctgaggtc agcctgaacg tggacactcc |
| 1741 | atttccactg gaagaggagc tgcagattga acccctgagc ctggatggac tcaacatgtt |
| 1801 | aagtgactcc agcatgggcc tgctggaccc ctctgttgaa gagacgtttc gagctgacag |
| 1861 | actgtgaaca gaaggcagtg gaacagaaga atgtttttct gcaacagcca aaatagaatg |
| 1921 | gaatagaatg aagccagctg ataccacggg ctttcgttat cttgacatag aaggaagcag |
| 1981 | tgccacggct ccagggtttc agatgagatc ccatctcaga cactgtggct tcctccagat |
| 2041 | cacacagctt tgtactgcct ctcccgcctg tggccaaagt cgtgttgcag caggcaggct |
| 2101 | gcttggagct tcccatgaac tggaaagctc acctccactg catcttttta ctggccatcc |
| 2161 | agtcagccga tgtgtaagag taggaaatac tgtgtcactg gaggccctcc gtagcattgg |
| 2221 | g |

In the sequence shown in Table 5, the coding sequence extends from position 8 to position 1867.

The polypeptide sequence predicted for the TORC3 protein based on the nucleotide sequence of Table 3 is shown in Table 6 (GenBank Acc. No. AAQ98858.1).

TABLE 6

(SEQ ID NO:6)

| | |
|---|---|
| 1 | maaspgsgsa nprkfsekia lhtqrqaeet rafeqlmtdl tlsrvqfqkl qqlrltqyhg |
| 61 | gslpnvsqlr snasefqpsf hqadnvrgtr hhglverpsr nrfhplhrrs gdkpgrqfdg |
| 121 | safganyssq pldeswprqq ppwkdekhpg frltsalnrt nsdsalhtsa lstkpqdpyg |
| 181 | gggqsawpap ymgfcdgenn ghgevasfpg plkeenllnv pkplpkqlwe tkeiqslsgr |
| 241 | prscdvgggn afphngqnlg lspflgtlnt ggslpdltnl hystplpasl dttdhhfgsm |
| 301 | svgnsvnnip aamthlgirs ssglqssrsn psiqatlnkt vlssslnnhp qtsvpnasal |
| 361 | hpslrlfsls npslsttnls gpsrrrqppv spltlspgpe ahqgfsrqls stsplapypt |
| 421 | sqmvssdrsq isflpteaqa qvsppppypa pqeltqpllq qprapeapaq qpqaasslpq |
| 481 | sdfqllpaqg ssltnffpdv gfdqqsmrpg pafpqqvplv qqgsrelqds fhlrpspysn |
| 541 | cgslpntilp edsstslfkd lnsalaglpe vslnvdtpfp leeelqiepl sldglnmlsd |
| 601 | ssmglldpsv eetfradrl |

As used herein and in the claims, the terms “TORC polynucleotide”, “TORCX polynucleotide”, where “X” takes on the values 1, 2 or 3, and similar terms and phrases, relate generally to the nucleotide sequences shown in Tables 1, 3 and 5, or their complements, as well as to a polynucleotide encoding a polypeptide whose amino acid sequence is at least 80% identical, or at least 85% identical, or at least 90% identical, or at least 95% identical, or at least 97% identical, or at least 98% identical, or at least 99% identical, to an amino acid sequence given in Tables 2, 4, or 6; or to a nucleotide sequence that is a fragment of any of the nucleotide sequences described in this paragraph; or to a nucleotide sequence that hybridizes to any nucleotide sequence described in this paragraph. In addition a TORC polynucleotide refers to a polynucleotide described above in this paragraph wherein the aforesaid fragment encodes a mature form of the polypeptide. A TORC polynucleotide further relates to polynucleotide described in above in this paragraph, or its complement, wherein a variant amino acid residue in the encoded polypeptide is a non-essential or a conservative substitution. A TORC polynucleotide further relates to a polynucleotide encoding a fusion protein, wherein the polynucleotide comprises a first TORC polynucleotide as described above in this paragraph and a second polynucleotide encoding a second polypeptide fused to the 5′ end or to the 3′ end of the first polynucleotide.

As used herein and in the claims, the terms “TORC polypeptide”, “TORCX polypeptide”, where “X” takes on the values 1, 2 or 3, and similar terms and phrases relate generally to the amino acid sequences shown in Tables 2, 4, and 6, as well as to a polypeptide whose amino acid sequence is at least 80% identical, or at least 85% identical, or at least 90% identical, or at least 95% identical, or at least 97% identical, or at least 98% identical, or at least 99% identical, to an amino acid sequence given in Tables 2, 4, or 6; or to a polypeptide whose amino acid sequence is a fragment of any of the amino acid sequences described in this paragraph. In addition a TORC polypeptide refers to a polypeptide described above in this paragraph wherein the aforesaid fragment encodes a mature form of the polypeptide. A TORC polypeptide further relates to polypeptide described in above in this paragraph, wherein a variant amino acid residue in the encoded polypeptide is a non-essential or a conservative substitution. A TORC polypeptide further relates to a polypeptide encoding a fusion protein, wherein the polypeptide comprises a first TORC polypeptide as described above in this paragraph and a second polypeptide encoding a second polypeptide fused to the 5′ end or to the 3′ end of the first polypeptide.

### EXAMPLES

### Materials and Methods

The following materials and methods are employed in the Examples described below.

Reagents, equipment, and culture conditions. All cell-lines are grown at 37° C. in a humidified incubator with 5% CO₂ in Dulbecco's Modified Eagle Medium (Invitrogen, Carlsbad, Calif. # 11995-065) supplemented with 10% fetal bovine serum, penicillin/streptomycin (Invitrogen #15140-122), and MEM non-essential amino acids (Invitrogen# 11140-050). Leptomycin B, ionomycin, rapamycin, and cyclosporin A are obtained from EMD Biosciences Inc., San Diego Calif. Phorbol 12-myristate 13-acetate (PMA; Sigma#P3766)), cyclopiazoic acid (Sigma#C1530), forskolin (Sigma#F3917), 3-isobutyl-1-methylxanthine (IBMX), dibutyryl cyclic-AMP (db-cAMP) are obtained from Sigma, St. Louis, Mo. Isoproterenol (cat#195263) is obtained from MP Biomedicals, Inc., Irvine, Calif. FK506 is obtained from Novartis (CGP048123-NX1, Novartis-Basel 81402076). DNA transfections are performed using Fugene6 transfection reagent (Roche Diagnostics, Indianapolis, Ind.), and siRNA transfections are performed using Oligofectamine (Invitrogen #12252-011) following the respective manufacturer's protocol. Cells are exposed to 1 joule/cm2 of 254 nm UV light in a UV Stratalinker 2400 (Stratagene, La Jolla, Calif.). The luciferase reporters pCRE-Luc, pNFAT-TA-Luc, and pNFKB-Luc are obtained from BD Biosciences, Inc., San Jose, Calif. The cDNA collection used in the translocation screen contain approximately 7,000 full-length human and murine cDNA's obtained from the Mammalian Genome Collection (MGC), Genomics Institute of the Novartis Research Foundation (available from the American Type Culture Collection, Manassas, Va.).

Nucleotide Sequences of TORC cDNAs. The nucleotide sequences encoding TORC proteins are available at GenBank accession nos. AY360171 (hTORC1), AY360172 (hTORC2), and AY360173 (hTORC3).

Plasmid construction and virus production. The FLAG-TORC1 expression plasmid, pCMV-FLAG-TORC1, is generated by PCR amplification of the human TORC1 coding region using the primers

(SEQ ID NO:7)

5′-GTA AAG CTT ATG GCG ACT TCG AAC AAT CCG-3′,
and

(SEQ ID NO:8)

5′-CGT GGA TCC TCA GTC CAT CCG GAA GGT GTC CTC-3′

and cloning this product into the HinDIII and BamHI sites of pFlag-CMV4 (Sigma).

The TORC1-eGFP expression plasmid, pCMV-TORC1-eGFP, is generated by PCR amplification of the human TORC1 coding region using the primers

(SEQ ID NO:9)

5′-CGC GAG ATC TAT GGC GAC TTC GAA CAA TCCG-3′,
and

(SEQ ID NO:10)

5′-ATA GGA TCC GTC CAT CCG GAA GGT GTC CTC-3′

and cloned into the BglII and BamHI sites in pEGFP—N1 (BD Biosciences). The TORC2-eGFP expression plasmid, pCMV-TORC2-eGFP, is generated by PCR amplifying human TORC2 coding region with the primers:

(SEQ ID NO:11)

5′ TTC TTT CGC TAG CGA GGC GAC GTC GGG GGC GAA CGG
GCCT 3′
and

(SEQ ID NO:12)

5′ GAA CTG CAG AAT TCG TTG GAG CCG GTC ACT GCG GAA
TGA 3′

and the resulting product is cloned into the NheI and EcoRI sites of pEGFP-N1 (U-4153 p35-55). The TORC3-eGFP expression plasmid, pCMV-TORC3-eGFP, is generated by PCR amplification of the TORC3 coding region using the primers

(SEQ ID NO:13)

5′ TTC TTT CGC TAG CGA TGG CCG CCT CGC CGG GCT CGG
GCA 3′
and

(SEQ ID NO:14)

5′ GAA CTG CAG AAT TCG CAG TCT GTC AGC TCG AAA CGT
CTC 3′

and the resulting PCR product is cloned into the NheI and EcoRI sites of pEGFP-N1 (U-4153 p35-55). The dominant negative TORC1 expression vector, pTORC1(1-44)-eGFP, is generated by PCR amplification of the coding region of TORC1 encoding the first 44 amino acids with primers

(SEQ ID NO:15)

5′ TCC CTT GCT AGC GCC ACC ATG GCG ACT TCG AAC AAT
CCG CGG AAA 3′
and

(SEQ ID NO:16)

5′ CTT TCT CAG AAT TCG CTG GAG CCG CGC GGC CCG CGT
CAG GCT 3′

and cloning the resulting product into the NheI and EcoRI sites of pEGFP-N1 (U-4153 p 116-117). The constitutively active calcineurin expression construct pCI-neo-CnA*-HA (Molkentin et al., 1998) is obtained from Eric Olson, University of Texas Southwestern Medical Center, Dallas Tex.

cDNA screen. HeLa cells are seeded in 384-well plates at 1,200 cells/well in 30 ul media 24 h prior to transfection. For each batch of seven 384 well plates, 35 ug pCMV-TORC1-eGFP is diluted into 10 ml OPTI-MEM and 562 ul Fugene6 is then added. 3 ul of this mixture is added to the cDNA stamps that contained 4 ul of approximately 7.5 ng/ul cDNA expression plasmid in OPTI-MEM, and finally the entire mix is added to the HeLa cells. After 48 hours, media is removed from the plates, and the plates are submerged in 100% methanol at −20 C for 20 minutes, the methanol is removed, and the plates are dried at room temperature. Nuclei are stained with 25 ul of 5 ug/ml Hoechst stain in PBS per well for 15 minutes followed by 3 washes of PBS. Cell images for each well are obtained using the Cellomics ArrayScan II and the amount of TORC1-GFP in the cytoplasm and the nucleus is determined using the Cytoplasm to Nuclear Translocation algorithm.

Generation of stable TORC-eGFP cell lines. HeLa cells are transfected with pCMV-TORC1-eGFP and stable integrants are selected with 700 ug/ml geneticin. A single colony that expressed TORC1-eGFP is isolated and expanded (HhTORC1eGFP cells).

HEK293 cells are transfected with either pCMV-TORC1-eGFP, pCMV-TORC2-eGFP or pCMV-TORC3-eGFP linearized with AseT restriction endonuclease and stable integrants are selected with 700 ug/ml geneticin generating a population of cells that express TORC-eGFP. These are referred to as 293hTORC1, 293hTORC2 or 293hTORC3 cells, respectively. All of the stable TORC-eGFP cell-lines are subjected to fluorescence activated cell sorting to remove non-fluorescing cells.

Confocal microscopy. Four-well glass slides seeded with HhTORC1eGFP cells are co-transfected with 0.38 ug pCMV-SPORT6 or respective cDNA plasmid along with 0.13 ug p3x-FLAG-CMV-7-BAP (Sigma#C-7472) as a transfection control. Media is replaced after 24 hours, and after 48 hours cells are fixed with 4% formaldehyde in PBS for 10 min at room temperature and permeabilized with 0.2% Triton X-100 in PBS for 2 min. Cells are blocked with 3% BSA in PBS for 1 hour and incubated with anti-FLAG monoclonal antibody (Sigma# F-3165) followed by staining with anti-mouse AlexaFluor 647 antibody (Molecular Probes, Eugene Oreg., Cat. #A-21463) and Hoechst 33342 stain (Molecular Probes #H3570).

TORC antibodies. Peptide antigens are generated and used to immunize rabbits to generate polyclonal antibodies which are then affinity purified against the original peptide. The antigens used for each antibody are:

anti-TORC1-PAS2769-2770:

| | |
|---|---|
| CSPHRRPLSVDKHGR; | (SEQ ID NO:17) |
| anti-TORC1-1B: | |
| ENPGQPSMGIDIASC; | (SEQ ID NO:18) |
| anti-TORC1-2A: | |
| CPATEDTFRMDRL; | (SEQ ID NO:19) |

anti-TORC1-EPO31350:

| | |
|---|---|
| KQAWDTKKTGSRPKSC; | (SEQ ID NO:20) |
| and | |

anti-TORC2-1cKSCN:

| | |
|---|---|
| CDPAVEESFRSDRLQ. | (SEQ ID NO:21) |

Anti-TORC1-EP031350 is generated by Eurogentec North America Inc., San Diego, Calif.; anti-TORC1-PAS2769-2770 is generated by ProSci Inc., Poway C A, and the remaining antibodies are generated by Zymed Laboratories, Inc., San Francisco Calif.

Immunohistochemistry. HEK293 derived cells are treated with either DMSO or 50 uM forskolin for 90 minutes, fixed with 100% methanol at −20° C. for 20 minutes, followed by drying and blocking with 5% BSA/10% goat serum/0.1% Tween-20 in PBS for 3 hours at room temperature. Cells are incubated with 1:4000 dilution of anti-TORC2 antibody 1cKSCN. Cells are then stained with anti-rabbit AlexaFluor 488 (Molecular Probes #A21441) and Hoechst stain prior to microscopy.

siRNA transfections. Six thousand HeLa cells are concurrently seeded and transfected with 90 ng pCRE-luc or pNFKB-luc and 10 ng Renilla luciferase plasmid with 0.3 ul Fugene6 per well in a 96-well plate. siRNA transfections are performed 24 hours later using Oligofectamine transfection reagent (Invitrogen) with a final concentration of siRNA of 20 nM in media lacking antibiotics, and media was exchanged to media containing antibiotics 24 hours after siRNA transfection. Media containing various compounds is exchanged 48 h after siRNA transfection, and luciferase activities are determined after an overnight incubation with the compounds. siRNA's used are:

nonspecific control:
(SEQ ID NO:22; Dharmacon, Dallas TX,
#D-001210-01-05)
5′-UAG CGA CUA AAC ACA UCA AUU-3′;
GL3 luciferase
(SEQ ID NO:23; Dharmacon#D-001400-01-05)
5′-CTT ACG CTG AGT ACT TCG A-3′;
TORC1
(SEQ ID NO:24; Dharmacon#D-014026-03)
5′-CCG GCA ACC UCG CGG CCA AUU-3′;
TORC2
(SEQ ID NO:25; Dharmacon#D-018947-02)
5′-CGA CUA CCA UCU GCA CUU AUU-3′;
and
TORC3
(SEQ ID NO:26; Dharmacon#D014210-04)
5′-CAA CGC AUC UGC UCU UCA CUU-3′.

Drosophila Methods. The Gal4 responder construct, UAS-GFP-TORC, is created by PCR cloning the eGFP gene from the pEGFP—N1 vector (Clontech # 6085-1) and the dTORC gene from Drosophila genomic DNA into the pUAST vector (Brand and Perrimon, 1993). This construct is injected into Drosophila embryos by P-element germline transformation to create transgenic lines (Rubin and Spradling, 1982). To induce expression of the fusion protein, flies containing UAS-GFP-TORC are crossed to flies containing the HS-Gal4 construct obtained from the Bloomington Stock Center, University of Indiana, Bloomington, Ind. The larvae are heat-shocked at 25° C. and salivary glands from late third instar larvae were dissected in PBS, placed in Sang M3 media (Sigma #S3652) containing various chemical treatments in a 96-well plate. The glands are visualized using the Nikon Eclipse TE2000-E fluorescent microscope. Images are taken at a 100× magnification.

### Example 1

### Subcellular Localization of TORC1 Protein

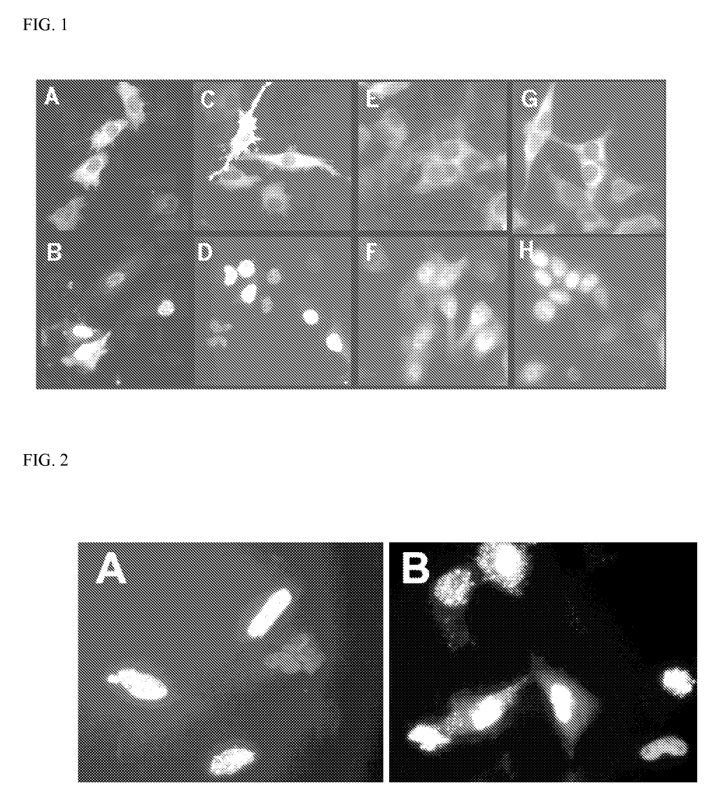

HeLa cells are transfected with FLAG-TORC1 (FIG. 1, panels A and B) or TORC1-eGFP (panels C and D). Cells are either untreated (panels A and C) or treated with 10 nM leptomycin-B (LMB), a fungal inhibitor of CRM1 mediated nuclear export of proteins, for 90 min (panels B and D). FLAG-tagged protein is visualized by immunofluorescence while eGFP-tagged proteins are directly visualized by fluorescence. Although TORC proteins have been shown to be CREB1-coactivators (Tourgenko et al., 2003), protein immunofluorescence using the FLAG epitope is found to be present predominantly in the HeLa cytoplasm (FIG. 1, panel A). Similar results are also obtained using direct fluorescence from eGFP with a TORC1eGFP fusion protein (TORC1-eGFP; FIG. 1, panel C). Since a variety of signal transduction proteins are regulated by either nuclear export or stimulus induced import, the localization of TORC1 is examined after treatment with LMB. After 90 minute exposure to LMB the TORC1 fusion proteins are found to be present predominantly in the nucleus (FIG. 1, panels B and D, respectively). Each of the tagged constructs appeared to retain the ability to activate CRE-dependent transcription similar to the untagged TORC constructs (data not shown).

### Example 2

### Subcellular Localization of TORC2 and TORC3 Proteins

The localization of human TORC2 and TORC3 is also examined. Both HeLa cells and HEK293 cells are transfected with either pCMV-TORC2-eGFP or pCMV-TORC3-eGFP. When expressed as eGFP fusions, TORC2 and TORC3 occurred constitutively in the nucleus in HeLa cells (FIG. 2). However, when expressed in HEK293 cells both TORC2- and TORC3-eGFP fusions proteins are largely, though not exclusively, present in the cytoplasm (FIG. 1, panels E and G, respectively, which show fluorescence primarily outside the nucleus). As with TORC1 in HeLa cells (Example 1), LMB treatment induces accumulation of both proteins in the nuclei of the cells, which now appear bright with fluorescence (FIG. 1, panels F and H, respectively). It may be concluded that the subcellular localization of the three human TORCs is regulated by nuclear export. (The localization of the TORC proteins in Examples 1 and 2 does not appear to be an artifact of either transfection or the labels. First, translocation and export are similar whether TORC1 is expressed with an N-terminal FLAG epitope tag or with a C-terminal eGFP label. Second, no translocation is seen after transfection with eGFP alone (data not shown). Finally, as described below, no treatment is found that resulted in appearance of translocation of a cytoplasmic alkaline phosphatase gene (Examples 4 and 7).

### Example 3

### Identifying cDNA's that Induce TORC1 Nuclear Accumulation

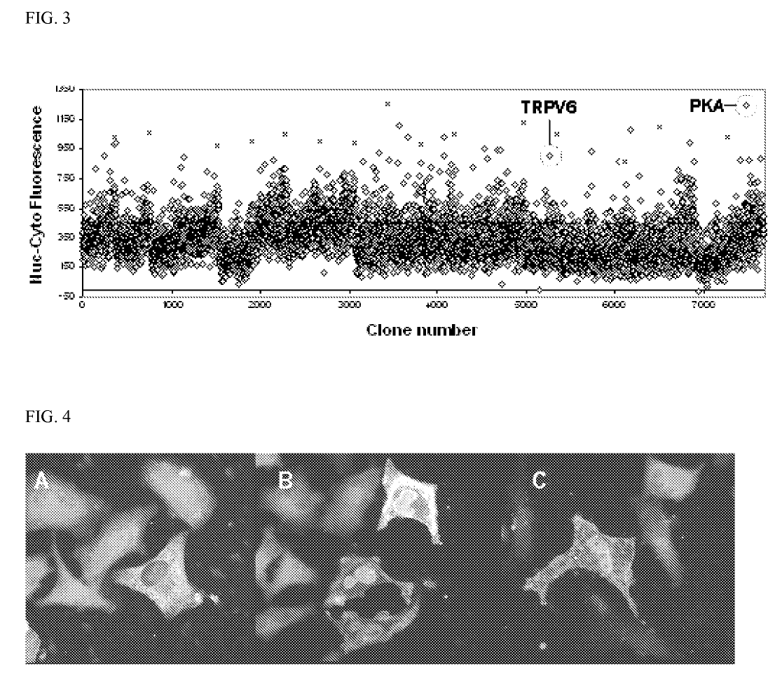

To identify cellular signals that regulate TORC translocation, a high-complexity screen is developed to identify genes that cause TORC1-eGFP to accumulate in the nucleus. HeLa cells are cotransfected with TORC1-eGFP in combination with approximately 7,000 individual cDNA expression constructs from the MGC Full-length collection (Strausberg et al., 2002). Forty-eight hours after transfection, the cells are fixed and the relative amounts of eGFP fluorescence in the cytoplasm and the nucleus are determined using the Cellomics ArrayScan II automated microscope. To provide a positive control for TORC1-eGFP translocation, one well in each 384-well cell-culture plate is treated with LMB prior to fixation. Translocation is monitored by plotting the nuclear-cytoplasmic fluorescence difference. Translocation via LMB is detected in virtually all control wells (FIG. 3, “X”). Potentially active cDNA's are recovered from clones with high nuclear-cytoplasmic fluorescence differences and retested in secondary assays. The highest scoring reproducible hits from this screen are the plasmids encoding the murine transient receptor potential cation channel, subfamily V, member 6 (TRPV6: cDNA clone MGC:27673 IMAGE:4911355) and a murine cAMP dependent protein kinase, catalytic, alpha subunit (PKA: cDNA clone MGC:6169 IMAGE:3497908). It should be noted that several other cDNAs are also confirmed for their ability to induce translocation of the tagged TORC1 protein.

### Example 4

### Confirmation of the Roles of TRPV6 and PKA

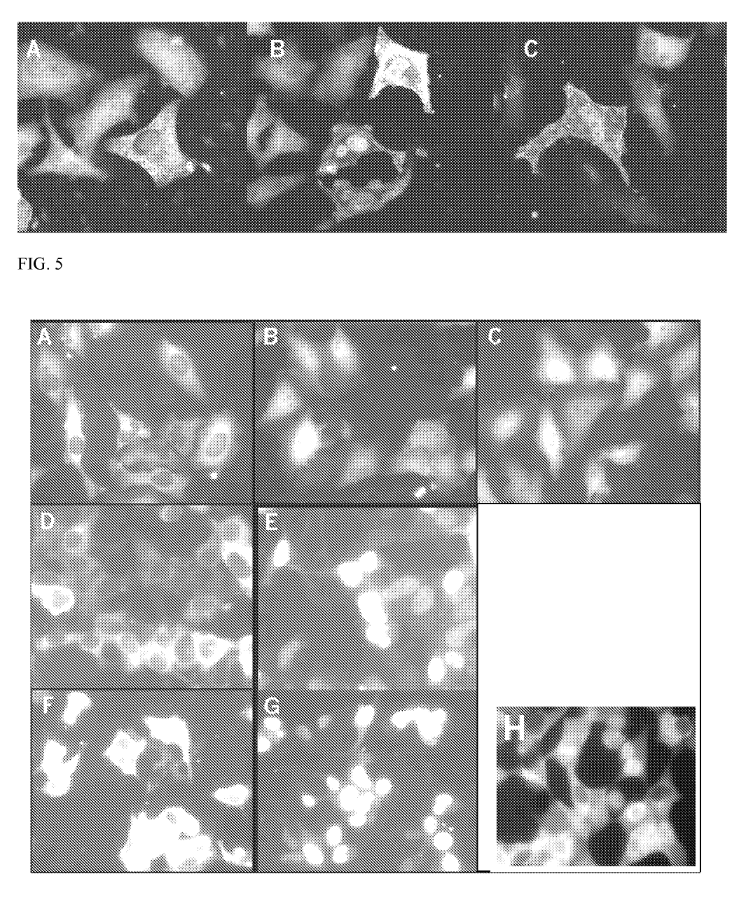

To confirm that TRPV6 and PKA induce TORC1-eGFP translocation to the nucleus (Example 3), HeLa cells stably expressing TORC1-eGFP are transfected with the empty expression vector pCMV-SPORT6 (FIG. 4, panel A), or the expression vector containing TRPV6 (panel B) or PKA (panel C). Confocal microscopy is carried out; in the original color photomicrographs alkaline phosphatase staining (pink) marks transfected cells, nuclear DNA staining is in blue, and TORC1-eGFP fluorescence is in green. In FIG. 4, the control using empty vector (panel A) shows one cell (lower right) with pink cytoplasm, indicating successful transfection. This cell has blue nuclear stain and slight green at or adjacent the nucleus (lower right portion). In panel B (TRPV6) the three cells on the right have cytoplasmic pink stain, and of these, the upper two have clearly green nuclei; the lower cell has a blue-green nucleus. In panel C (PKA) the large cell in the center has pink cytoplasm, and has a green nucleus. These results show that the gene products of both these cDNA clones induce nuclear accumulation of TORC1-eGFP. TRPV6 resulted in virtually all the TORC1-eGFP moving to the nucleus while PKA resulted in only partial accumulation of TORC1-eGFP in the nucleus. These observations demonstrate that translocation of TORC1 into the nucleus is a regulated event, and suggest that TORC activity may be induced by cAMP and calcium signal transduction pathways.

Both TRPV6 and PKA also induce translocation of TORC2 and TORC3 in HEK293 cells, however only PKA is sufficiently active to induce nuclear accumulation of TORC2 and TORC3 without simultaneously blocking nuclear export with LMB (Example 5).

### Example 5

### Dependence of Translocation of TORC Proteins on Camp

Induction of TORC1 translocation by PKA suggests that cAMP might coordinately activate CREB via phosphorylation and TORC via nuclear transport. In order to assess this possibility, HeLa cells stably expressing TORC1-eGFP are exposed to either no treatment (FIG. 5, panel A), 50 uM forskolin and 100 uM IBMX (panel B), or 1 mM db-cAMP (panel C) for 2 hours. It is found that increasing intracellular cAMP concentrations by these treatments results in significant appearance of eGFP fluorescence in the nucleus (panels B and C), indicating partial translocation of TORC1-eGFP in HeLa cells compared to the no-treatment control which exhibits only cytoplasmic fluorescence. (FIG. 5, panel A). Similarly, HEK293 cells stably expressing TORC2-eGFP (FIG. 5, panels D and E) or TORC3-eGFP (panels F and G) are either untreated (panels D and F) or treated with 25 uM forskolin for 1 hour (panels E and G). It is seen from the fluorescence microscopy results in panels E and G that in stably transfected HEK293 cells TORC2-eGFP and TORC3-eGFP are translocated to the nucleus by treatment with forskolin alone.

In addition, both TORC2-eGFP (data not shown) and TORC3-eGFP (FIG. 5, panel H) accumulate in the nucleus in stably transfected HEK293 cells cotransfected with PKA.

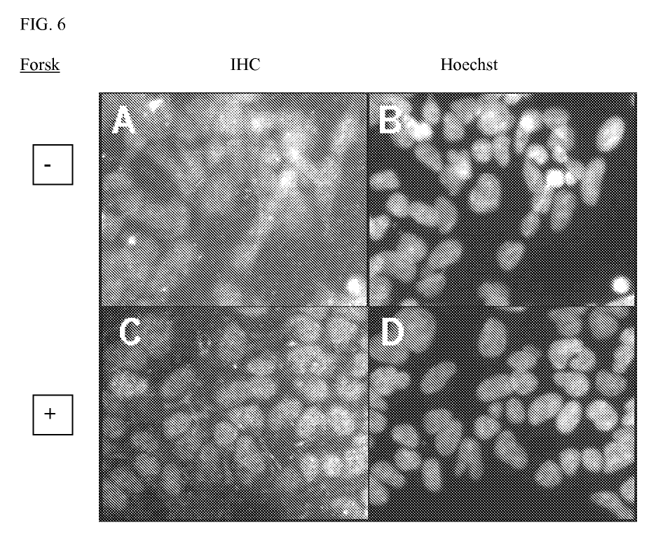

The localization of endogenous TORC2 is examined in wild type HEK293 cells by immunohistochemical staining following forskolin exposure. HEK293 cells are either untreated (FIG. 6, panels A and B), or treated with 50 uM forskolin for 90 min (panels C and D). Anti-TORC2 staining is shown panels A and C, with the corresponding nuclear Hoechst staining in panels B and D. Untreated cells display diffuse staining throughout the cell while cells exposed to forskolin display predominantly nuclear immunohistochemical staining indistinguishable from that seen when the nuclei are stained with Hoechst. Thus, the subcellular localization of all the TORCs appears to be regulated by intracellular cAMP levels.

It should be noted that assessing the translocation of endogenous TORC proteins is difficult due to the low level of expression of the proteins and the properties of the antibody reagents available. Although the antibody produced to date easily recognizes overexpressed protein, the TORC1 antibody can recognize endogenous protein with low sensitivity and TORC3 antisera have failed to detect endogenous protein.

### Example 6

### Stimulation of Translocation of TORC by G-Protein Coupled Receptors

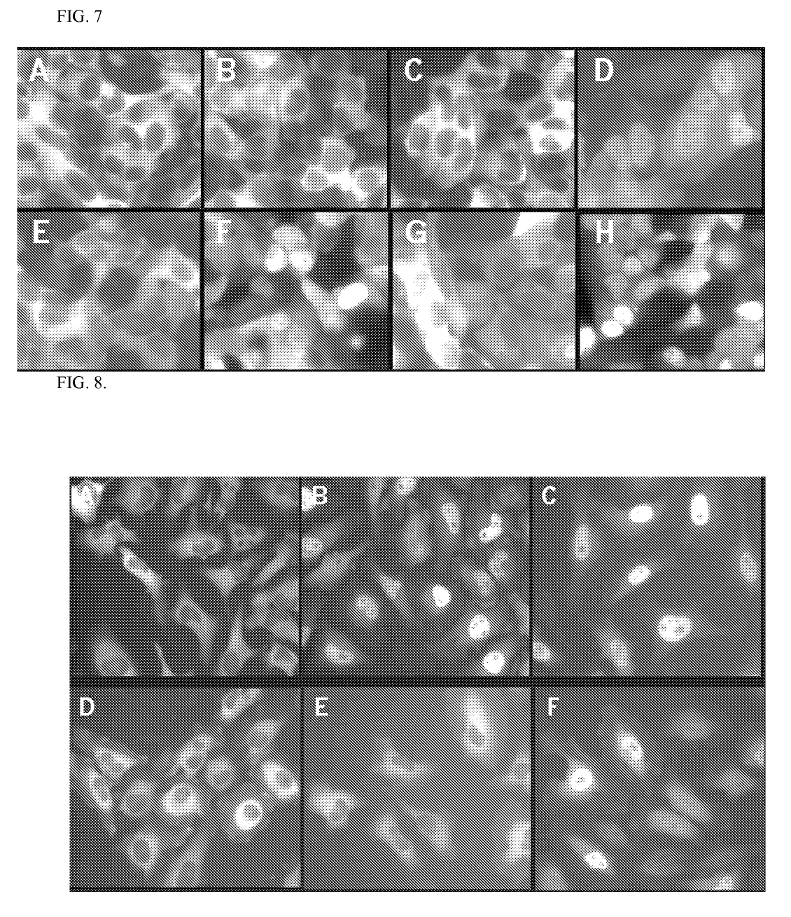

To assess whether TORC translocation is induced by stimulation of endogenous G-protein coupled receptors (GPCRs), HEK293 cells stably expressing various TORC-eGFP fusions are treated with isoproterenol, a β2-adrenergic receptor agonist, for 1 hr. As shown in FIG. 7, untreated HEK293 cells stably expressing TORC1-eGFP (panel A), and cells treated with 10 nM LMB alone (panel B) or 160 nM isoproterenol alone (panel C) show eGFP fluorescence originating from the cytoplasm. Only treatment with isoproterenol plus LMB (panel D) results in fluorescence emanating primarily from the nucleus.

In the case of HEK293 cells stably expressing TORC2-eGFP or TORC3-eGFP, fluorescence from the nucleus occurs with treatment by 160 nM isoproterenol alone (FIG. 7, panels F and H, respectively), compared to cytoplasmic fluorescence with untreated cells (panels E and G, respectively). Thus translocation of TORC1-eGFP to the nucleus in response to isoproterenol treatment occurs only in the presence of LMB, while isoproterenol alone is sufficient for nuclear translocation of both TORC2-eGFP and TORC3-eGFP.

In contrast to HeLa cells, the import of TORC1-eGFP is slow in stably transfected HEK293 cells and required both LMB and forskolin and IBMX or isoproterenol treatment (data not shown), suggesting the need for both an import signal and blockade of export.

### Example 7

### TORCs are Translocated in Response to Calcium in a Calcineurin Dependent Manner

TRPV6 is a calcium channel that is suspected, but not proven, to be a store operated calcium channel involved in capacitative calcium entry (CCE) and calcium signaling (Cui et al., 2002). This is particularly intriguing as CCE is required to activate and induce nuclear translocation of another transcription factor, Nuclear Factor of Activated T-cells (NF-AT; see Hogan et al., 2003 for review). NF-AT contains a nuclear localization signal that is inactivated by phosphorylation; nuclear import of NF-AT requires dephosphorylation by the calcium-dependent phosphatase, calcineurin. NF-AT also contains a calcineurin-regulated nuclear export sequence (NES) (Zhu and Mckeon 1999). Calcineurin is the target of immunosuppressive drugs cyclosporine A (CsA) and FK506 which potently block stimulus dependent transport of NF-AT to the nucleus.

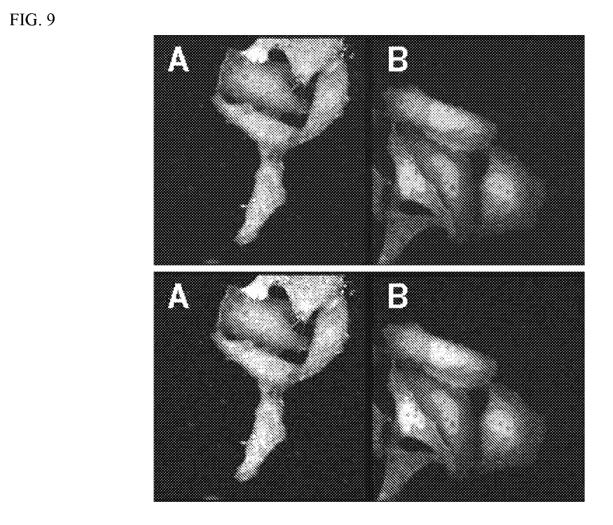

To determine whether translocation of TORCs is also regulated by the level of intracellular calcium, localization of TORC proteins in response to the calcium ionophore ionomycin, or to the inhibitor of the sarcoplasmic-endoplasmic reticulum calcium ATPase cyclopiazonic acid (CPA) is examined (FIG. 8). HeLa cells stably expressing TORC1-eGFP are exposed to either DMSO carrier (panel A), 1 uM ionomycin (panel B), or 10 uM CPA (panel C) for 1 hour. Both ionomycin and CPA potently induce accumulation of the TORC1-eGFP in the nucleus, as seen by the eGFP fluorescence, compared to cytoplasmic fluorescence seen with DMSO alone.

Panels D, E, and F also show treatment with 1 uM ionomycin. Upon preexposure to 6.4 nM FK506 (panel D) or 5 uM CsA (panel E) for 1 hour the eGFP fluorescence remains in the cytoplasm, indicating that translocation in response to ionomycin is completely blocked by these agents. Treatment with 2 uM rapamycin, an immunophillin-binding compound that does not inhibit calcineurin, (panel F) does not block translocation, as seen by strong nuclear fluorescence. The fact that translocation in response to ionomycin is completely blocked by both CsA and FK506, but not by rapamycin, suggests that calcineurin is implicated in calcium-induced translocation.

The role of calcineurin in TORC translocation is further tested by examining localization of TORC1-eGFP after transfection of an activated form of calcineurin (FIG. 9). HeLa cells stably expressing TORC1-eGFP are transiently transfected with either an empty expression vector (panel A) or a plasmid expressing a constitutively active form of calcineurin (panel B). In panel A the four lower cells show blue nuclei and green cytoplasm tinged with pink (staining the for alkaline phosphatase transfection control). All the cells in panel B shows slight pink stain in the cytoplasm and strong green fluorescence, overwhelming the blue nuclear stain, in the nucleus, for all cells. Thus expression of active calcineurin induces accumulation of TORC1-eGFP in the nucleus, mimicking the effect of TRPV6 or ionomycin. Cotransfection of activated calcineurin in HEK293 cells is also found to induce translocation of both TORC2 and TORC3 (data not shown).

### Example 8

### TRPV6 Regulates Translocation of TORC

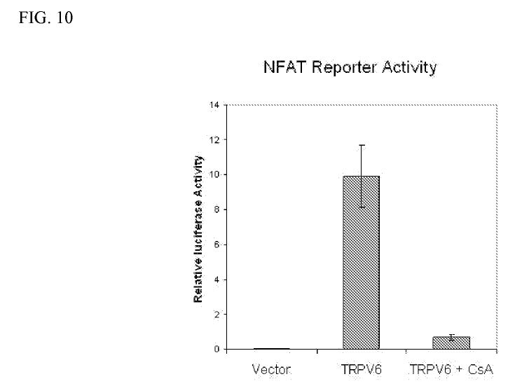

To determine whether TRPV6 expression is sufficient to also activate NF-AT signaling, HEK293 cells are transiently transfected with either empty vector or TRPV6 expression vector in combination with an NF-AT dependent luciferase reporter. 3 uM CsA is added 18 hours prior to the reporter assay. It is found that TRPV6 activates NF-AT as revealed by luciferase (FIG. 10). This activation is inhibited by CsA, consistent with the hypothesis that TRPV6 activates NF-AT via activation of calcineurin. Thus TORC translocation is regulated in a manner similar to NF-AT and appears to represent a novel target of calcineurin and the immunophillin-binding immunosuppressants. It is believed that this is the first demonstration that TRPV6 expression is sufficient to activate calcineurin and thus, that it may play a role in T-cell activation.

### Example 9

### Calcium Dependent Activation of TORC Translocation

The effect of calcium-signaling on the nuclear translocation of the three human TORC-eGFP fusion proteins is examined in stably expressing HEK293 cells. In response to LMB exposure, TORC2-eGFP and TORC3-eGFP do accumulate in the nucleus, yet at a rate that is much slower than in HeLa cells. TORC3-eGFP becomes predominantly nuclear approximately 90 min following LMB exposure, and TORC2-eGFP becomes predominantly nuclear by 120 min following LMB exposure. In HeLa cells, on the other hand, TORC1 is translocated to near completion in less than 30 minutes. (data not shown).

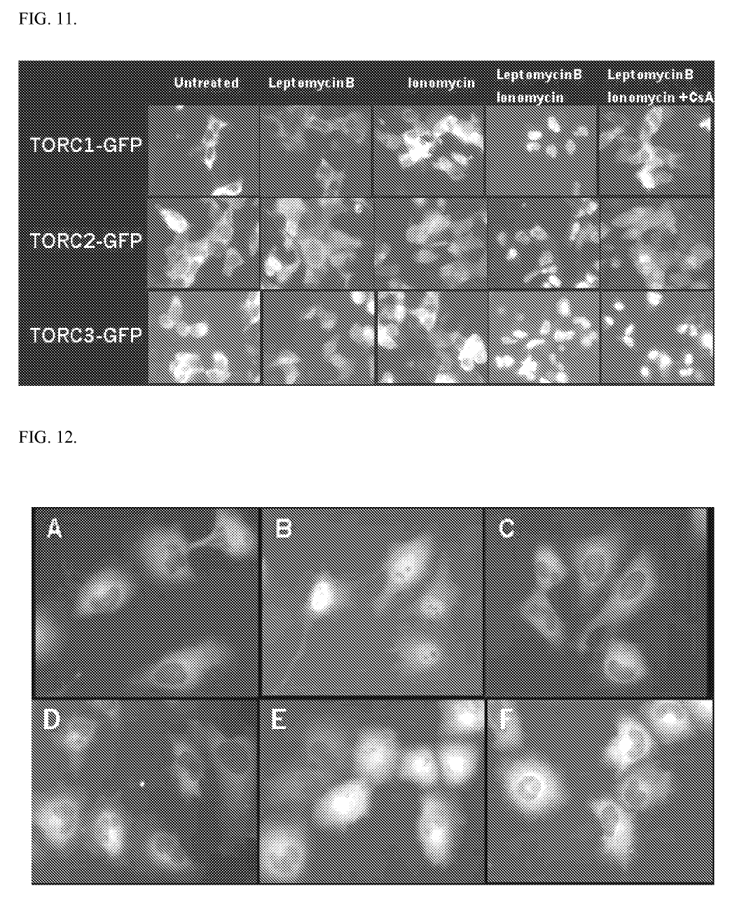

HEK293 cells stably expressing TORC1-eGFP, TORC2-eGFP, or TORC3-eGFP are exposed to 10 nM LMB, 10 uM ionomycin, 10 nM LMB and 10 uM ionomycin, or 10 nM LMB, 10 uM ionomycin, and 5 uM CsA (FIG. 11). Treatments are for 45 min and the CsA treatment includes a 15 min pre-exposure to 1 uM CsA. As shown by fluorescence microscopy of eGFP, when the cells are exposed solely to ionomycin or LMB for 45 minutes, each of the three TORC's exhibit weak or no translocation to the nucleus in HEK293 cells. But the combination of both ionomycin and LMB results in efficient translocation of all three TORC-eGFP fusion proteins into the nucleus (FIG. 11). Therefore, nuclear translocation induced by calcium in HEK293 cells appears to require both a positive signal as well as inhibition of nuclear export. Interestingly, the three TORC's differ in their sensitivity to CsA. Nuclear accumulation of TORC1-eGFP is completely blocked by CsA whereas TORC2- and TORC3-eGFP proteins are partly or unaffected by CsA respectively (FIG. 11). Thus, although all three TORCs translocate in response to calcium signaling, the mechanisms regulating TORC2 and TORC3 translocation may be distinct from that of TORC1, at least in HEK293 cells. It should be noted that the kinetics of nuclear import and export may explain the different requirements for combination of inducers and LMB to accumulate different TORCs in the nucleus.

### Example 10

### Dependence of TORC Translocation on Other Stimuli

The effect of several other stimuli that result in CREB phosphorylation and CRE-dependent transcription also result in the coordinate induction of TORC translocation. HeLa cells stably expressing TORC1-eGFP are untreated (FIG. 12, panels A and D) or exposed to UV (1 joule/cm2) alone (panel B) or UV in the presence of 10 uM CsA (panel C), 10 uM PMA (panel E), or 10 uM PMA and 5 uM CsA (panel F). Images are obtained 10 min after UV exposure or 1 hour after PMA exposure. Both UV irradiation of cells (FIG. 12, panel B compared with panel A) and the protein kinase C (PKC) agonist phorbol 12-myristate-13acetate (PMA) induced TORC1-eGFP to accumulate in the nucleus (panel E compared with panel D). Translocation by both UV and PMA are blocked by CsA treatment (panels C and F, respectively), demonstrating a requirement for calcineurin in both of these responses. TORC translocation in response to cAMP is insensitive to CsA (data not shown) revealing that at least two independent pathways modulate the subcellular localization of the TORC proteins.

### Example 11

### TORC nuclear translocation is sufficient for induction of CRE-mediated gene expression

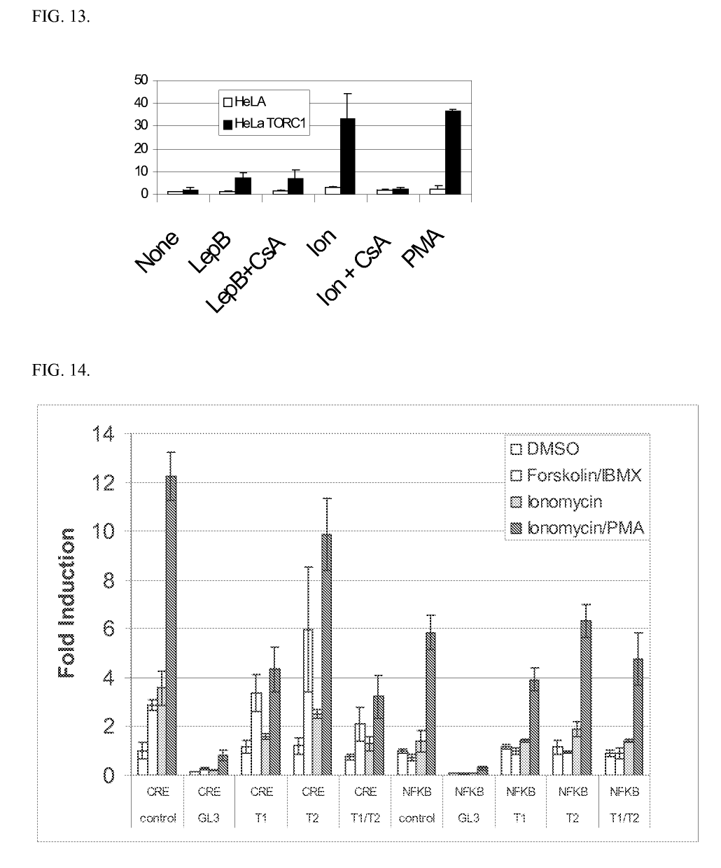

To determine the effect of TORC1 over-expression and localization on gene expression, CRE-dependent transcription is compared in untransfected HeLa cells and HeLa cells stably expressing TORC1-eGFP using a luciferase reporter containing a CRE-dependent promoter. HeLa cells and HeLa cells stably expressing TORC1-eGFP are stimulated with 10 nM LMB, 10 uM CsA, 5 uM ionomycin, 10 uM PMA or the indicated combinations for 18 hours (FIG. 13). Both western analysis and real-time PCR analysis indicate that HeLa cells express TORC1, but at extremely low levels, while the TORC1-eGFP stable cell-line produces significantly more TORC1 protein (data not shown). As shown in FIG. 13, while untreated naïve HeLa cells and transfected HeLa::TORC1-eGFP cells display similar levels of CRE-driven luciferase, LMB treatment resulted in a significant induction only in the HeLa::TORC1-eGFP cells. This suggests that movement of the TORC1-eGFP to the nucleus was sufficient to activate CRE-driven gene expression. In addition, while ionomycin and PMA resulted in a modest level of activation in naïve HeLa cells, these agents showed greater than 10-fold larger increases in CRE-luciferase induction in HeLa::TORC1-eGFP cells. Further, the induction by ionomycin is completely blocked by CsA. Activation by PMA was partially blocked by CsA (data not shown), suggesting that PMA may induce TORC activity both calcineurin dependent and independent mechanisms. Thus, over-expression of TORC1 potently enhanced activation of CRE driven gene expression, and movement of the exogenous TORC1 to the nucleus by LMB is sufficient for induction of CRE-driven gene expression. Thus, over-expression of TORC1 potently enhanced activation of CRE driven gene expression in a calcineurin dependent fashion. It is believed that these observations provide a mechanism to explain previous findings that CsA and FK506 inhibit a expression of a subset of CREB responsive genes (Siemann et al. 1999).

### Example 12

### Knockdown of TORC's Using siRNAs

To determine if TORC function is essential for activation through calcium signaling, TORC1 and TORC2 expression is inhibited with siRNAs. siRNAs that significantly block expression of human TORC1 and TORC2 protein are identified by western blot analysis using TORC1 and TORC2 specific antibodies (data not shown). HeLa cells are transiently transfected with either a CRE- or NFKB-luciferase plasmid prior to transfection with 20 nM siRNA specific for luciferase (GL3), TORC1 (T1), TORC2 (T2) or a combination of both TORC1 and TORC2 (T1/T2), vs. a control nonspecific siRNA. Each cotransfected cell preparation is treated with either DMSO, 50 uM forskolin and 100 uM IBMX, 10 uM ionomycin, or 10 uM ionomycin with 10 uM PMA 48 hours after siRNA transfection (FIG. 14). The anti-pGL3 siRNAs are used as a positive control for siRNA transfection and efficiency.

Knockdown of either TORC1 or TORC2 alone is sufficient to decrease CRE-activation through ionomycin. The siRNA to TORC1 alone inhibit induction by PMA and ionomycin whereas the TORC2 siRNA has little effect. When both TORC1 and TORC2 siRNAs are transfected, the greatest inhibitory effect is observed against all stimuli. These effects are specific as the TORC siRNAs had little effect on induction of an NF-kappaB dependent luciferase reporter by ionomycin and PMA (FIG. 14), nor had any effect on the basal expression of an SV40 driven Renilla luciferase reporter (data not shown).

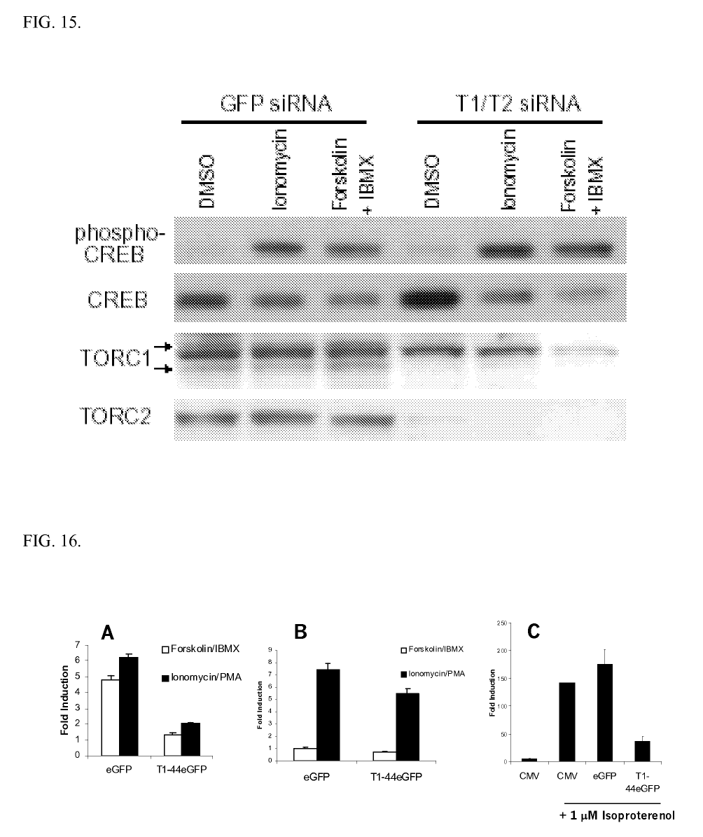

HeLa cells are either transfected with either anti-GFP specific siRNA or both TORC1 and TORC2 specific siRNA's prior to induction with DMSO, ionomycin, or forskolin and IBMX for 20 min followed by lysis. The siRNAs are shown to block TORC1 and TORC2 protein production by western blot analysis (FIG. 15). It should be noted that forskolin/IBMX induction of CRE-expression is only modestly blocked by TORC1 and TORC2 siRNAs. The weak inhibition of forskolin/IBMX induction might be due to either insufficient blockade by the siRNAs or due to the expression of all three human TORC proteins in HeLa cells. More complete inhibition of CRE-activation is observed when siRNAs to TORC3 are also included (data not shown). In those experiments it was not possible to confirm inhibition of TORC3 protein expression due to the lack of antibody of sufficient quality. Blockade of TORC1 and TORC2 expression has no effect on CREB1 phosphorylation by either ionomycin or forskolin/IBMX (FIG. 15), consistent with previous data that TORCs activates CRE-driven expression independently of CREB-phosphorlyation.

The experiments reported in this Example show that TORCs are essential for calcium induced CRE-driven transcription.

### Example 13

### Dominant Negative Mutant Constructs of TORC

The siRNA results (Example 12) are complicated by the need to use multiple siRNAs and to block multiple TORCs. In order to efficiently block activity of all TORC proteins more readily, a dominant interfering TORC protein is designed which fuses the highly conserved 44 amino-acid CREB-binding domain of TORC1 to eGFP (T1-44eGFP). To block activity of all TORC proteins efficiently, a dominant interfering protein that should specifically block the ability of all TORC's to interact with CREB1 is designed. The CREB1 interaction domain of TORC proteins is present in the first 44 amino acids of TORC1. The N-terminal 44 amino acids, representing the first exon of the TORC1 gene, is nearly 100% conserved in virtually all mammalian, C. elegans and Drosophila TORC proteins, yet has virtually no homology or similarity to any other proteins (Tourgenko et al., 2003). When these 44 amino acids are expressed as a fusion protein with eGFP (T1(1-44)eGFP), the enhanced CRE-response in HhTORC1 cells is effectively blocked and the activity of all three TORCs on CRE-activation is inhibited (data not shown). HeLa cells are transfected with either the eGFP control or TORC1(1-44)-eGFP with either CRE-luciferase (FIG. 16, panel A) or NFKB-luciferase (panel B) prior to stimulation with 50 uM forskolin/100 uM IBMX or 5 uM ionomycin. In panel C, HEK293 cells are transiently transfected with CRE-luciferase and the β2-adrenergic receptor (β2AR) in combination with either the empty CMV vector, eGFP, or TORC1(1-44)-eGFP fusion and exposed to isoproterenol for 18 hours. The dominant negative T1(1-44)eGFP also potently blocks CRE activation through either forskolin/IBMX or ionomycin and PMA (FIG. 16, panel A). This activity is specific as there is no effect on expression by eGFP alone and T1(1-44)eGFP has no effect on activation of an NF-kappaB reporter by ionomycin and PMA (FIG. 16, panel B). As shown in FIG. 16, panel C, T1(1-44)eGFP, but not eGFP strongly inhibits CRE-luciferase induction by the β2-adrenergic receptor (β2AR) and isoproterenol. Thus blockade of TORC function also prevents CRE-activation through a Gs-linked GPCR using isoproterenol. It may be concluded that this demonstrates TORC function is both sufficient and essential for activation of a CRE response.

### Example 14

### TORC Translocation is an Evolutionarily Conserved Mechanism of Regulation

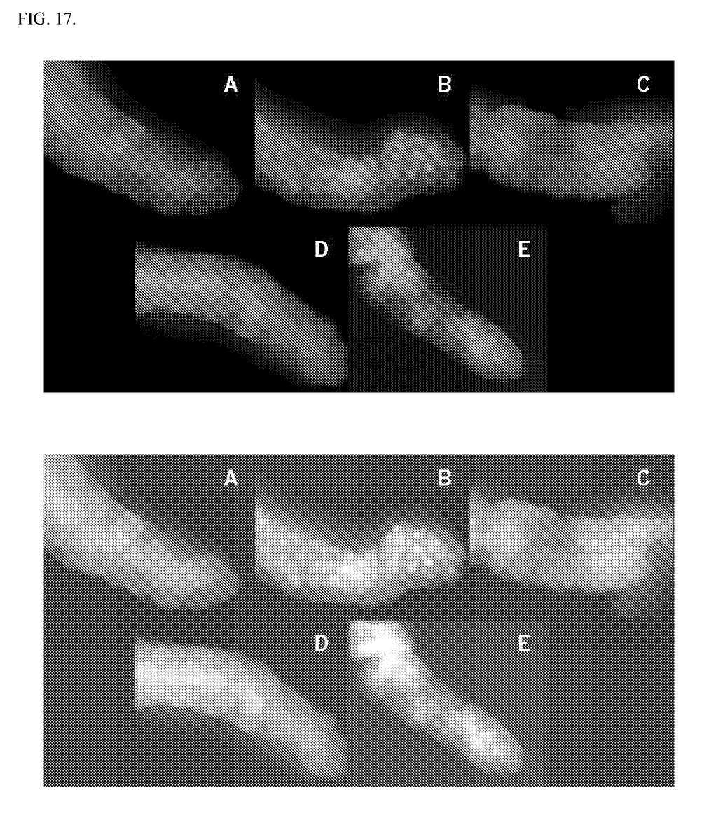

A fusion of eGFP and the Drosophila TORC (dTORC) protein is generated. Drosophila larval salivary glands expressing the dTORC-eGFP transgene were subjected to the following treatments: untreated for 1 hour (FIG. 17, panel A); exposed to 5 uM ionomycin for 1 hour (panel B); 5 uM ionomycin with 10 uM CsA for 1 hour (panel C); untreated for 4 hours (panel D); 100 uM db-cAMP for 4 hours (panel E). Salivary glands dissected from transgenic larvae expressing the dTORC fusion protein are examined by microscopy. dTORC-eGFP is localized in the cytoplasm in all of the cells in the gland (FIG. 17, panels A and D). Exposure of the transgenic salivary glands to either ionomycin or db-cAMP induced translocation of dTORC-eGFP into the nucleus (panels B and E, respectively). The ionomycin-induced translocation is blocked with CsA (panel C). Thus, the localization of TORC in Drosophila is regulated by cAMP and calcium, just as in human cells. Thus, this regulation of TORC subcellular localization within the cell is a highly conserved mechanism of modifying CREB signaling.

### Example 15

### Subcellular Localization of TORC Proteins in Neuronal Cells

A culture of a human brain cell line chosen from HCN-1A (cortical neuron), HCN-2 (cortical neuron), DBTRG-05MG (glial cell; glioblastoma), or rat PC-12 cells induced to exhibit a neuronal phenotype by nerve growth factor, (all available from ATCC) or murine primary dorsal root ganglion explant neurons (Araki et al. (2004) Science 305:1010-1013) is established. The cells are transfected with either pCMV-TORC1-eGFP, pCMV-TORC2-eGFP or pCMV-TORC3-eGFP. After 24-48 hours the eGFP localization is examined using fluorescence microscopy. The results are reexamined in the presence of LMB. These results characterize the subcellular localization of TORC proteins in neuronal cells.

### Example 16

### Identifying an Agent that Modulates the Activity of a TORC-Related Process in a Brain Cell

A neuronal cell or phenotypic neuronal cell (Example 15) is established in culture and transfected with either pCMV-TORC1-eGFP, pCMV-TORC2-eGFP or pCMV-TORC3-eGFP. The culture is divided in two portions, only one portion of which is treated with a candidate TORC-affecting agent. Typically, a library, such as a combinatorial library, of candidates exists. In multiwell plates, portions of the transfected neuronal cell culture are treated with various members of the library. In order to select agents with the most potent modulating activity, the culture may be pretreated with a substance known to promote accumulation of a TORC protein in the cytoplasm, or with a substance known to promote accumulation of a TORC protein in the nucleus. The concentration of the substance may be adjusted so that it is just at or near a midpoint of effectiveness in promoting its effect. The subcellular localization of the TORC protein-eGFP fusion is examined using fluorescence microscopy, and compared with the portion of the original culture, also treated with the substance but not treated with the candidate agent. A candidate agent that induces a significant effect in comparison with the untreated portion of the culture is identified as a biologically effective agent according to this assay.

General Methods: The following methods and substances were used in the experiments set out in Examples 17 to 23.

Cell culture: Primary muscle cells were isolated from 2 to 3 wk-old FVB male mice. Myoblasts were cultured in Ham's F10 (Gibco, cat# 11550-043) containg 20% heat-inactivated FBS (Hyclone, cat# 30070.03), 100 U/ml penicillin-100 μg/ml streptomycin (Gibco, cat# 15140-122), 1× Normocin (Invivogen, cat# ant-nr-1) and 2.5 ng/ml bFGF (Invitrogen, cat# 13256-029). When they reached 80% confluency, they were induced to differentiate by switching to DMEM (Gibco, cat# 11965-092) containing 5% horse serum (Hyclone, cat# SH30074).

Transfection and luciferase activity measurement: HeLa cells were grown in DMEM containing 10% FBS. They were seeded on a 96 well plate at 7000 cells per well and transfection was performed 6 hrs later using Fugene 6 transfection reagent (Roche, Cat# NC916732). The amount of DNA and Fugene 6 used for tranfections was 163 ng and 0.325 μl, respectively. Cells were lysed 48 or 72 hr post-transfection and subjected to luciferase activity measurement using the Dual-Glo luciferase assay system (Promega, Cat# PRE2943-0) according to the supplier's instructions.

Transduction with lentiviruses or adenoviruses: HeLa cells were seeded at 1.3×10⁵ per well on 6 well plates. 2 ml of medium containing 0.5 ml TORC1 or STOP (control) lentivirus, 8 mg/ml polybrene (Sigma, Cat# H9268) and 10 mM Hepes was added to each well the plates were centrifuged at 1000 g for 30 min to increase transduction efficiency. The medium was removed and changed to DMEM with 10% FBS 24 hrs later. Cells were harvested at 24, 48 or 72 hr post-transduction. Primary muscle cells (day 1 post-differentiation) were infected with adenovirus of TORC2, TORC3 or GFP (control) (4×10⁸ particle per well on a 6 well plate). Medium was changed daily. Cells were harvested 24 or 48 hr post-transduction.

RNA extraction and real-time PCR analysis of gene expression: Total RNA was extracted from cells using Trizol (Invitrogen, Cat# 15596-026). 1 mg of total RNA was used for cDNA synthesis performed with Superscript II RNase H Reverse Transcriptase (Invitrogen, Cat# 18064-022). The relative expression of the target genes was determined by real-time PCR using the synthesized cDNA with primer/probe sets specific to the target genes. The relative mRNA expression levels were calculated comparing the target genes/18S rRNA. The Taqman probe sequence of cytochrome oxidase subunit II (CoxII) is 5′-TCAAGCAACAGTAACATCAAACCGACCA-3′. (SEQ ID NO: XX) The Taqman primer sequences of CoxI are 5′-CCATCCCAGGCCGACTAA-3′ (SEQ ID NO: XX) (forward) and 5′-CAGAGCATTGGCCATAGAATAACC-3′ (SEQ ID NO: XX) (reversed). The probe and primers for CoxII were obtained from Sigma Genosys and Applied Biosystems, respectively. The Taqman primer/probe sets for other genes were obtained from Applied Biosystems (listed below):

Human and mouse cytochrome c (Hs01588973_ml; Mm01621048_s1)
Human and mouse PGC-1α (Hs00173304_m1; Mm00447183 m1)

### Mouse ERRα (Mm00433143_m1)

Mouse isocitrate dehydrogenase (Mm00499674_m1)

### 18S (Cat# 4310893E)

Protein extraction and Western blotting: Cells were lysed using Cell extraction buffer (Biosurce, Cat# FNN0011) supplemented with Complete Mini protease inhibitor cocktail (Roche, Cat# DCTC0). 50 μg of cell lysate was used for Western blot analysis. TORC1 antibody was obtained from Mark Labow (FGA) and used at 1:2000 dilution. Antibodies against tubulin (Abcam, Cat# ab3194) and V5 (Invitrogen, Cat# 960-25) were used at 1:3000 and 1:5000, respectively. The 2^{nd} antibodies, goat-anti-rabbit IgG (Cat#31460) and goat-anti-mouse IgG (Cat# 31430) were purchased from Pierce and used at 1:10,000 dilution.

Fatty acid oxidation: Cells were labeled with 36 μM ¹⁴C-palmitate (ARC, Cat# ARC-172A) in a non-carbonate buffer containing 114 mM NaCl, 4.7 mM KCl, 1.2 mM KH₂PO₄, 1.2 mM MgSO₄, and 0.5% fatty acid-free BSA for 2 hrs. ¹⁴CO₂ produced by the cells was quantified and served as index of fatty acid oxidation rate.

Cellular respiration: Cells were trysinized and resuspended in PBS containing 25 mM glucose and 1 mM pyruvate and 2% fatty acid free BSA. Cellular respiration was measured without treatment and with treatment of oligomycin and CCCP using a Clark electrode. 3 million of HeLa cells or 0.5 million of primary muscle cells was used for each measurement. The concentration of oligomycin was 2 μg/ml and the concentration of CCCP was 2 μM and 2-5 μM for HeLa cells and primary muscle cells, respectively.

### Example 17

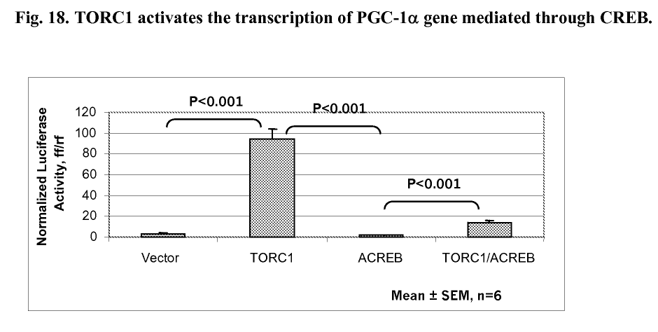

Ectopic expression of TORC1 increases mitochondrial gene expression and mitochondrial oxidative capacity in HeLa cells. FIG. 18 illustrates the results when HeLa cells were transfected with pGL3-basic-2 kb-PGC1α-Luc, phRL-SV40 (as control for transfection efficiency) along with various cDNA constructs as indicated. Cells were lyses and luciferase activity was determined 48 hrs post-transfection. A ratio of firefly luminescence (ff) over Renilla luminescence (RL) was calculated (mean±SEM) and expressed as normalized luciferase and used as an index of PGC-1α gene transcription. TORC1 induces transcription of the PGC-1α promoter as demonstrated by reporter protein expression. The presence of ACREB, a dominant negative inhibitor of CREB, decreased the effect of TORC1, thus illustrating that TORC1 operates through a CREB mechanism.

### Example 18

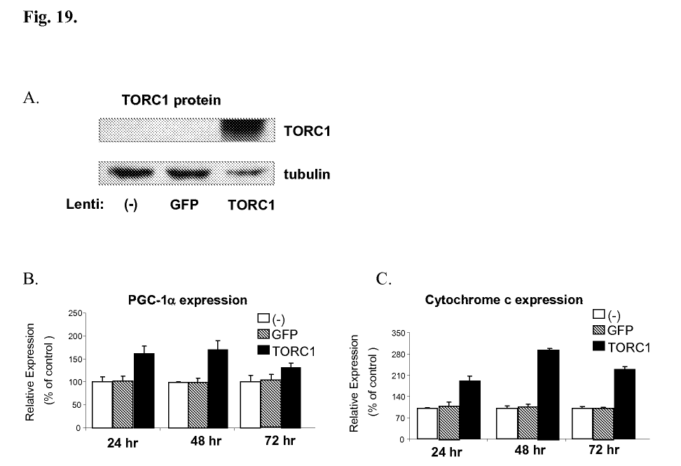

FIG. 19 demonstrates that ectopic expression of TORC1 increases the expression of PGC-1α and cytochrome c suggesting that it induces mitochondriogenesis. Cytochrome c is an accepted biomarker of mitochondrial levels. HeLa cells were transduced with either the control lentiviruses (stop or GFP) or TORC1 lentivirus. A. Total protein was extracted from cells 72 hr post-transduction and subjected to Western blot analysis to determine the expression of PGC-1α protein. B. Total RNA was extracted from cells at 24, 48 and 72 hr post-transduction and subjected to qPCR analysis to determine the expression level of endogenous PGC-1α (B) and cytochrome c (C) mRNAs.

### Example 19

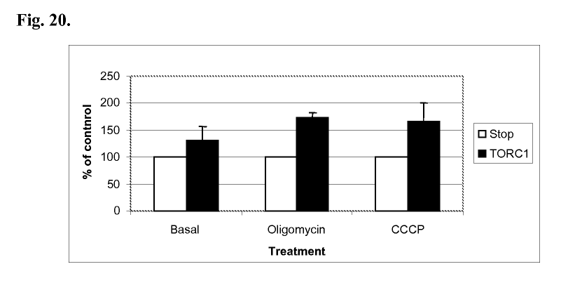

FIG. 20 demonstrates that ectopic expression of TORC1 increases cellular respiration in HeLa cells. HeLa cells were transduced with either the control (Stop) or TORC1 lentivirus. Cellular respiration was determined using a Clark electrode 72 hr post-transduction. The concentrations of Oligomycin (MP Biomedicals; ATP synthase inhibitor) and CCCP (Sigma; electron transport chain uncoupler) are 2 μg/ml and 2 μM, respectively. Increased cellular respiration is observed after TORC1 expression.

### Example 20

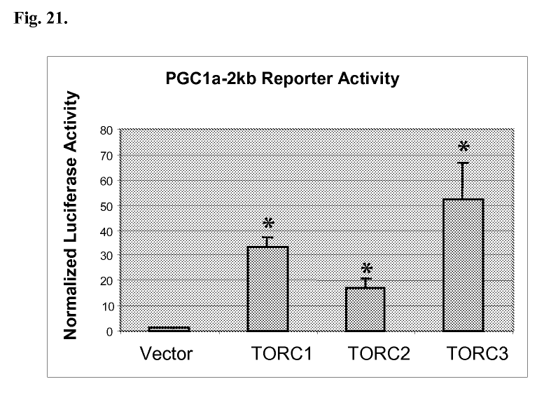

Ectopic expression of TORC2 and TORC3 increase mitochondrial gene expression and mitochondrial oxidative capacity in mouse primary muscle cells. FIG. 21. illustrates that each of TORC1, TORC2 and TORC3 activate the transcription of PGC-1α gene. HeLa cells were transfected with pGL3-basic-2 kb-PGC-1α-Luc, phRL-SV40 (as control for transfection efficiency) along with various cDNA constructs as indicated. Cells were lyses and luciferase activity was determined 48 hrs post-transfection. A ratio of firefly luminescence over Renilla luminescence was calculated (mean±SEM) and expressed as Normalized luciferase activity and used as an index of PGC-1α gene transcription. N=6, *P<0.05 for TORC1, 2, 3 vs. Vector.

### Example 21

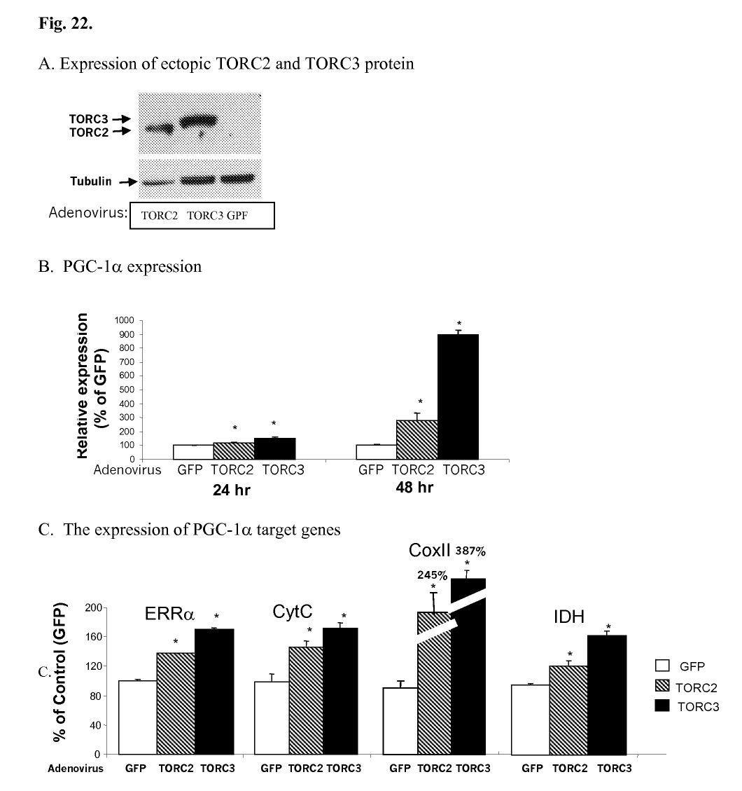

In FIG. 22, ectopic expression of TORC2 or TORC3 is shown to increase the expression of PGC-1α and mitochondrial markers in primary muscle cells. Primary muscle cells were transduced with either TORC2, TORC3 or GFP adenovirus. A. Total protein was extracted from cells 48 hr post-transduction and subjected to Western blot analysis to determine the expression of ectopic TORC2 and TORC3 protein. The primary and secondary antibodies to detect the ectopic TORC2 and TORC3 proteins were V5 and goat-anti-rabbit IgG, respectively. B-C. Total RNA was extracted from cells at 24 and 48 hr post-transduction and subjected to qPCR analysis to determine the expression level of endogenous PGC-1α (B) and PGC-1α target genes (C) including ERRα and mitochondrial markers, cytochrome C (CytC), cytochrome oxidase subunit II (CoxII), isocitrate dehydrogenase (IDH). N=3, *P<0.05 (TORC2 or TORC3 vs. GFP).

### Example 22

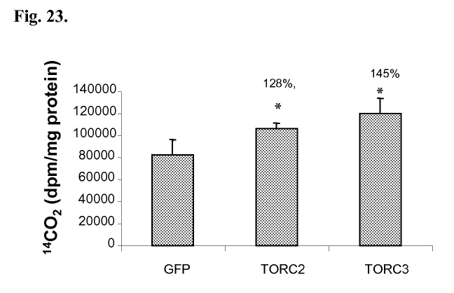

FIG. 23. shows that ectopic expression of TORC2 or TORC3 increased fatty acid oxidation in mouse primary muscle cells. Primary muscle cells were transduced with either TORC2, TORC3 or GFP adenovirus. 14C-palmitate oxidation was performed 48 hr post-transduction. N=3, *P<0.05. This functional assay further confirms the role of TORC proteins in mitochondrial biogenesis.

### Example 23

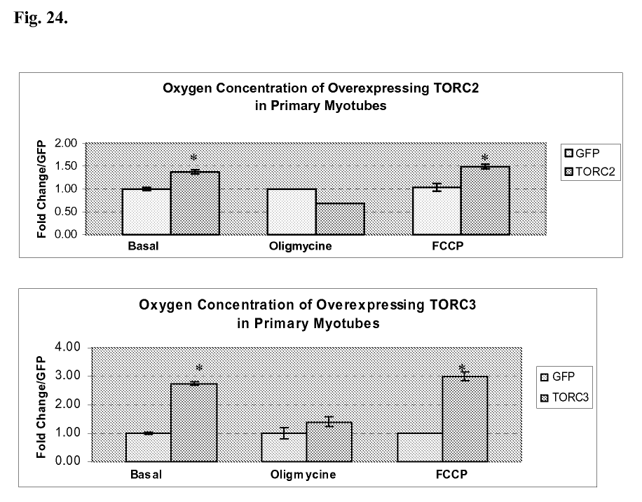

In FIG. 24, mouse primary muscle cells are shown to increase cellular respiration upon ectopic expression of TORC2 or TORC3. Primary muscle cells were transduced with either TORC2, TORC3 or GFP adenovirus for 48 hr. Cellular respiration was measured without treatment (basal), or oligomycin or CCCP treatment. N=3, *p<0.05 TORC vs. GFP.

The data presented in Examples 1-14 suggest that agonists of TORC activity, expression or nuclear translocation could by themselves induce or potentiate CREB-mediated processes in neurons. As cAMP and CREB mediated responses have been linked with a number of pathologic and physiologic processes, modifiers of TORC activity could have a variety of applications. TORC agonists could find utility in enhancing memory, treating depression, mood disorders or schizophrenia. Loss of CREB function has been linked to neurodegeneration and blockade of CREB function results in neuronal cell death and apoptosis. Further, in Huntington's disease, the precipitated nuclear inclusion bodies are thought to sequester CBP and thus would also block CREB activity (Sugars et al., 2003; Kazantsev 1999). Restoration of CREB-inducible gene expression by TORC agonists might again have clinical utility in this setting.

The Examples also identify an ability of TRPV6 to potently induce calcineurin dependent induction of NF-AT driven gene expression. As discussed above, TRPV6 is suspected of composing or being part of the store-operated calcium channel essential for activation of calcineurin in response to intracellular transient rises in calcium concentrations. The observations that TRPV6 can induce translocation of TORCs as well as NF-AT driven gene expression in a CsA sensitive manner, strongly supports a role for TRPV6 in NF-AT driven processes such as cardiac hypertrophy and T-cell activation.

The movement of TORCs to the nucleus provides an efficient cost effective general means to identify activators or inhibitors of cAMP or calcium mediated signal transduction events. A variety of assays are available to study the levels of cAMP in cell culture or activation of calcium mediated signaling. However, most of these methods which utilize reporter gene or ELISA type measurements are costly, time consuming and are difficult to apply to individual cells in a high throughput mode. Visualization of TORC translocation allows for identification of modifiers of cAMP and calcium signaling in individual cells in high-throughput.

Finally, as TORC1 translocation is shown to be sufficient to induce and enhance CREB-mediated expression, compounds that induce TORC nuclear-translocation may find benefit as memory enhancers, or as neuroprotective agents while compounds that block TORC nuclear-translocation could have application in treatments for arteriosclerosis.

### REFERENCES

Arias J, Alberts A S, Brindle P, Claret F X, Smeal T, Karin M, Feramisco J, Montminy M. 1994. Activation of cAMP and mitogen responsive genes relies on a common nuclear factor. Nature. 370:226-9.

Bodor, J., Bodorova, J., and Gres, R. E. (2000). Suppression of T cell function: a potential role for transcriptional reporessor ICER. Journal of leukocyte Biol. 67: 774-779.
Bourtchuladze, R., Frenguelli, B., Blendy, J., Cioffi, D., Schutz, G., and Silva, A. J. (1994). Deficient long-term memory in mice with a targeted mutation of the cAMP-responsive element-binding protein. Cell 79, 59-68.
Brand, A. H. and Perrimon, N., 1993. Targeted gene expression as a means of altering cell fates and generating dominant phenotypes. Development 118: 401-415.
Brindle P, Nakajima T, Montminy M. 1995. Multiple protein kinase A-regulated events are required for transcriptional induction by cAMP. Proc Natl Acad Sci USA. 92:10521-5.
Chao J, Nestler E J. (2004). Molecular neurobiology of drug addiction. Annu Rev Med. 2004; 55:113-32.
Chrivia J C, Kwok R P, Lamb N, Hagiwara M, Montminy M R, Goodman R H. 1993. Phosphorylated CREB binds specifically to the nuclear protein CBP. Nature 365:855-9.
Conkright M D, Canettieri G, Screaton R, Guzman E, Miraglia L, Hogenesch J B, Montminy M. TORCs: transducers of regulated CREB activity. Mol Cell; 12:413-23.
Cui J, Bian J S, Kagan A, McDonald TV. (2002). CaT1 contributes to the stores-operated calcium current in Jurkat T-lymphocytes. J Biol. Chem. 277:47175-83.
Deak, M., Clifton, A. D., Lucocq, L. M., and Alessi, D. R. (1998). Mitogen- and stress-activated protein kinase-1 (MSK1) is directly activated by MAPK and SAPK2/p38, and may mediate activation of CREB. Embo J 17, 4426-4441.
Du, K., and Montminy, M. (1998). CREB is a regulatory target for the protein kinase Akt/PKB. J Biol Chem 273, 32377-32379.
Gau D, Lemberger T, von Gall C, Kretz O, Le Minh N, Gass P, Schmid W, Schibler U, Korf H W, Schutz G. 2002. Phosphorylation of CREB Ser142 regulates light-induced phase shifts of the circadian clock. Neuron. 34:245-53.
Gonzalez, G. A., and Montminy, M. R. (1989). Cyclic AMP stimulates somatostatin gene transcription by phosphorylation of CREB at serine 133. Cell 59, 675-680.
Herzig, S., Long, F., Jhala, U., Hedrick, S, Quinn, R, Bauer, A., Rudolph, D, Schutz, G., ‘Yoon, C., Puigserver, P., Spiegelman, B. and Montminy, M. (2001). CREB regulates hepatic gluconeogenesis through the coactivator PGC-1. Nature, 413: 179-183.
Hogan P G, Chen L, Nardone J, Rao A. (2003). Transcriptional regulation by calcium, calcineurin, and NFAT. Genes Dev. 2003 Sep. 15; 17(18):2205-32.
Iourgenko V, Zhang W, Mickanin C, Daly I, Jiang C, Hexham J M, Orth A P, Miraglia L, Meltzer J, Garza D, Chirn G W, McWhinnie E, Cohen D, Skelton J, Terry R, Yu Y, Bodian D, Buxton FP, Zhu J, Song C, Labow M A. 2003. Identification of a family of cAMP response element-binding protein coactivators by genome-scale functional analysis in mammalian cells. Proc Natl Acad Sci USA. 2003 Oct. 14; 100(21):12147-52
Jackson T, Ramaswami M. (2003). Prospects of memory-modifying drugs that target the CREB pathway. Curr Opin Drug Discov Devel. 2003 September; 6(5):712-9.
Kazantsev A, Preisinger E, Dranovsky A, Goldgaber D, Housman D. (1999). Insoluble detergent-resistant aggregates form between pathological and nonpathological lengths of polyglutamine in mammalian cells. Proc Natl Acad Sci USA. 1999 Sep. 28; 96(20):11404-9.
Lonze, B., and Ginty, D. (2002). Function and Regulation of CREB Family Transcription Factors in the Nervous System. Neuron 35, 605.
Lonze, B., Riccio, A., Cohen, S., and Ginty, D. D. (2002). Apoptosis, axonal growth defects, and degeneration of peripheral neurons in mice lacking CREB. Neuron 34, 371-385.
Mantamadiotis, T., Lemberger, T., Bleckmann, S. C., Kern, H., Kretz, O., Martin Villalba, A., Tronche, F., Kellendonk, C., Gau, D., Kapfhammer, J., et al. (2002). Disruption of CREB function in brain leads to neurodegeneration. Nat Genet. 31, 47-54.
Mayr, B., and Montminy, M. (2001). Transcriptional regulation by the phosphorylation-dependent factor CREB. Nat Rev Mol Cell Biol 2, 599-609.
Molkentin J D, Lu J R, Antos C L, Markham B, Richardson J, Robbins J, Grant S R, Olson E N. (1998). A calcineurin-dependent transcriptional pathway for cardiac hypertrophy. Cell.; 93:215-28.
Moore M S, DeZazzo J, Luk A Y, Tully T, Singh C M, Heberlein U. (1998) Ethanol intoxication in Drosophila: Genetic and pharmacological evidence for regulation by the cAMP signaling pathway. Cell. 1998 Jun. 12; 93(6):997-1007.
Ono H, Ichiki T, Fukuyama K, Tino N, Masuda S, Egashira K, Takeshita A. (2002). Arterioscler Thromb Vasc Biol. 2004 Jul. 8 [Epub ahead of print].
Rubin, G. M. and Spradling, A. C., 1982. Genetic transformation of Drosophila with transposable element vectors. Science 218: 348-353.
Rudolph, D., Tafuri, A., Gass, P., Hammerling, G. J., Arnold, B., and Schutz, G. (1998). Impaired fetal T cell development and perinatal lethality in mice lacking the cAMP response element binding protein. Proc Natl Acad Sci USA 95, 4481-4486.
Shaywitz, A. J., and Greenberg, M. E. (1999). CREB: a stimulus-induced transcription factor activated by a diverse array of extracellular signals. Annu Rev Biochem 68, 821-861.
Siemann G, Blume R, Grapentin D, Oetjen E, Schwaninger M, Knepel W. (1999). Mol Pharmacol: 55, 1094-1100.
Strausberg, R. L., Feingold, E. A., Grouse, L. H., Derge, J. G., Klausner, R. D., Collins, F. S., Wagner, L., Shenmen, C. M., Schuler, G. D., Altschul, S. F. et al. (2002) Proc. Natl. Acad. Sci. U.S. A 99, 16899-16903.
Sugars K L, Brown R, Cook L J, Swartz J, Rubinsztein D C. (2003) Decreased cAMP response element-mediated transcription: an early event in exon 1 and full-length cell models of Huntington's disease that contributes to polyglutamine pathogenesis. J Biol. Chem. 2004 Feb. 6; 279(6):4988-99. Epub 2003 Nov. 18
Tully T, Bourtchouladze R, Scott R, Tallman J. (2003). Targeting the CREB pathway for memory enhancers. Nat Rev Drug Discov. 2003 April; 2(4):267-77.
Valverde O, Mantamadiotis T, Torrecilla M, Ugedo L, Pineda J, Bleckmann S, Gass P, Kretz 0, Mitchell J M, Schutz G, Maldonado R. (2004). Modulation of anxiety-like behavior and morphine dependence in CREB-deficient mice. Neuropsychopharmacology. 2004 June; 29(6): 1122-33.
West, A. E., Chen, W. G., Dalva, M. B., Dolmetsch, R. E., Kornhauser, J. M., Shaywitz, A. J., Takasu, M. A., Tao, X., and Greenberg, M. E. (2001). Calcium regulation of neuronal gne expression. Proc. Natl. Acad. Sci. 98:11024-11031.
Xing, J., Kornhauser, J. M., Xia, Z., Thiele, E. A., and Greenberg, M. E. (1998). Nerve growth factor activates extracellular signal-regulated kinase and p38 mitogen-activated protein kinase pathways to stimulate CREB serine 133 phosphorylation. Mol Cell Biol 18, 1946-1955.
Yin J C, Wallach J S, Del Vecchio M, Wilder E L, Zhou H, Quinn W G, Tully T. (1994). Induction of a dominant negative CREB transgene specifically blocks long-term memory in Drosophila. Cell; 79:49-58
Zhu J, McKeon F. (1999). NF-AT activation requires suppression of Crm1-dependent export by calcineurin. Nature. 1999 Mar. 18; 398(6724):256-60.

We claim:

1. A method to identify modulators of a CREB-promoted process in a cell comprising contacting the cell with a substance that modulates accumulation of a TORC polypeptide in a subcellular compartment of the cell.

2. The method described in claim 1 wherein the substance is an ionophore, a stimulator of intracellular cAMP concentration, a stimulator of intracellular calcium concentration, or a stimulator of a protein kinase activity.

3. A method of modifying a TORC-related process in a cell comprising contacting the cell with a substance that promotes accumulation of a TORC polypeptide in a subcellular fraction of the cell.

4. The method described in claim 3 wherein the substance is an immunophilin binding agent.

5. A method of modulating CREB-promoted processes in a cell comprising contacting the cell with a substance that modulates expression of a TORC polypeptide in the cell.

6. The method described in claim 5 wherein the substance comprises an antisense oligonucleotide, an interfering oligonucleotide, a microoligonucleotide, a triple helix nucleic acid, a ribozyme, an RNA aptamer, double or single stranded RNA, a peptidomimetic, a polynucleotide comprising a sequence encoding a peptidomimetic, or a mixture of any two or more of them.

7. A method of substantially inhibiting the development of, treating, or ameliorating a disease or pathological condition in a subject related to an abnormal level of a CREB-promoted process in a cell, comprising administering one or more therapeutically effective doses to the subject of a substance that modulates accumulation of a TORC polypeptide in a subcellular region of the cell.

8. The method described in claim 7 wherein the substance is an immunophilin binding agent.

9. The method described in claim 7 wherein the substance is an ionophore, a stimulator of intracellular cAMP concentration, a stimulator of intracellular calcium concentration, or a stimulator of protein kinase activity.

10. The method of claim 7 wherein said TORC modulator comprises one or more antibodies to a TORC protein, or fragments thereof, wherein said antibodies or fragments thereof inhibits the activity of said TORC protein.

11. The method of claim 7 wherein said TORC modulator comprises one or more peptide mimetics to a TORC protein wherein said peptide mimic inhibits the activity of said TORC protein.

12. A method of substantially inhibiting the development of, treating, or ameliorating a disease or pathological condition in a subject related to an abnormal level of a TORC-activated and CREB-promoted process in a cell, comprising administering one or more therapeutically effective doses to the subject of a substance that modulates expression of a TORC polypeptide in the cell.

13. The method of claim 12 wherein said modulatory substance comprises an antisense oligonucleotide, an interfering oligonucleotide, a microoligonucleotide, a triple helix nucleic acid, a ribozyme, an RNA aptamer, double or single stranded RNA, a peptidomimetic, a polynucleotide comprising a sequence encoding a peptidomimetic, or a mixture of any two or more of them.

14. A method of identifying an agent that modulates the activity of a CREB-promoted process in a cell, comprising
a) introducing a TORC polypeptide into a first cell and into a reference cell;
b) contacting the first cell with the agent; and
c) determining whether a biological function of the TORC polypeptide in the first cell has been modulated in comparison with the biological function of the TORC polypeptide in the reference cell that has not been contacted with the agent;
whereby if such a functional modulation occurs the modulating agent is identified.

15. The method of claim 14 wherein the biological function comprises modulating the distribution of the TORC polypeptide among a plurality of subcellular compartments.

16. A method of detecting the presence or quantifying the amount of a TORC polypeptide in a sample comprising the steps of:
a) providing a sample suspected of comprising a TORC polypeptide;
b) contacting the polypeptide with a specific binding agent that binds a TORC polypeptide under conditions that assure binding of the TORC polypeptide to the specific binding agent; and
c) detecting the presence or quantifying the amount of the specific binding agent that binds to the TORC polypeptide.

17. The method described in claim 16 wherein the sample is derived from a subcellular fraction of a cell.

18. A method of determining whether the amount of a TORC polypeptide in a sample differs from the amount of the TORC polypeptide in a reference, wherein the method comprises the steps of:
a) providing a sample suspected to include the TORC polypeptide;
b) contacting the sample with a specific binding agent that binds a TORC polypeptide under conditions that assure binding of the TORC polypeptide to the specific binding agent; and
c) determining whether the amount of the specific binding agent that binds to the sample differs from the amount of the specific binding agent that binds to a reference under the same conditions used in step b), wherein the reference comprises a standard or reference amount of the TORC polypeptide.

19. The method described in claim 18 wherein the sample is derived from a subcellular fraction of a cell.

20. A method of contributing to the diagnosis or prognosis of a disease or pathology in a first subject, wherein the subcellular localization of a TORC polypeptide in the pathology is known to differ from the subcellular localization of the TORC polypeptide in a nonpathological state, the method comprising the steps of:
a) providing a sample from the first subject suspected to include the TORC polypeptide;
b) contacting the sample with a specific binding agent that binds a polypeptide described in claim 16 under conditions that assure binding of the TORC polypeptide to the specific binding agent; and
c) determining whether the amount of the specific binding agent that binds to the sample differs from the amount of the specific binding agent that binds to a reference under the same conditions used in step b), wherein the reference is provided from a second subject known not to have the pathology;
thus contributing to the diagnosis or prognosis of, or to developing a therapeutic strategy for, the pathology.

21. The method described in claim 20 wherein the sample is derived from a subcellular fraction of a cell.

22. A method of identifying an agent that modulates TORC polypeptide activity in a cell, comprising
a. contacting a first cell with an agent;
b. contacting a reference cell with a control agent; and
c. determining whether a change in subcellular compartmentalization of a TORC polypeptide has occurred in the first cell compared to the reference cell;
wherein an agent which induces a change in subcellular compartmentalization of TORC polypeptide in the first cell relative to said reference cell is an agent that modulates TORC polypeptide activity in a cell.

23. The method of claim 22 wherein said TORC polypeptide is selected from among TORC1, TORC2 and TORC3.

24. The method of claim 22 wherein said change in subcellular compartmentalization is nuclear translocation.

25. The method of claim 22 wherein said first cell and said reference cell each comprise a recombinant TORC polypeptide.

26. Use of a compound first identified in the method of claim 22 as an agent that modulates TORC polypeptide activity for the treatment of a disease selected from among depression, mood disorders, schizophrenia, neurodegenerative conditions, Alzheimer's Disease, Parkinson's Disease and Huntington's disease, or as a neuroprotective agent or for enhancing memory.

27. Use of a compound first identified in the method of claim 22 as an agent that modulates TORC polypeptide activity for the treatment of a disease selected from among arteriosclerosis, osteoarthritis, psoriasis, asthma, chronic obstructive pulmonary disease (COPD), rheumatoid arthritis, cancer, pathological angiogenesis, diabetes, hypertension, chronic pain, inflammatory disease and autoimmune disease.

28. Use of compound first identified in the method of claim 22 as an agent that modulates TORC polypeptide activity in the manufacture of a medicament for the treatment of a disease selected from among depression, mood disorders, schizophrenia, neurodegenerative conditions, Alzheimer's Disease, Parkinson's Disease and Huntington's disease, arteriosclerosis, osteoarthritis, psoriasis, asthma, chronic obstructive pulmonary disease (COPD), rheumatoid arthritis, cancer, pathological angiogenesis, diabetes, hypertension, chronic pain, inflammatory disease and autoimmune disease or for use as a neuroprotective agent or for enhancing memory.
